SEQUENCE LISTING (16 sequences)

SEQ ID NO: 1 - DNA, 351 bp, Mus sp.
  CDS (1)..(351)
caggtgcagctggtggagtctgggggaggcgttgtgcagcctggaaggtcccatagactctcctgtgcagcctctggattcaccttcagtagctatgacatgtcttgggttcgccaggctccgggcaagggtctggagtgggtcgcaaaagttagtagtggtggtggtagcaccatcatattagacactgtgcagggccgattcaccatctccagagacaatagtaagaacaccctatacctgcaaatgaactctctgagagccgaggacacagccgtgtattactgtgcaagacataactacggcagttttgcttactggggccaagggactacagtgactgtttctagt

SEQ ID NO: 2 - protein, 117 aa, Mus sp.
QVQLVESGGGVVQPGRSHRLSCAASGFTFSSYDMSWVRQAPGKGLEWVAKVSSGGGSTIILDTVQGRFTISRDNSKNTLYLQMNSLRAEDTAVYYCARHNYGSFAYWGQGTTVTVSS

SEQ ID NO: 3 - DNA, 321 bp, Mus sp.
  CDS (1)..(321)
gagattgtgctaactcagtctccagccaccctgtctctcagcccaggagaaagggcgactctttcctgccaggccagcgaaagtattagcaaccacctacactggtatcaacaaaggcctggtcaagccccaaggcttctcatcaagtatcgttcccagtccatctctgggatccccgccaggttcagtggcagtggatcagggacagatttcaccctcactatctccagtctggagcctgaagattttgcagtctattactgtcaacagagtggcagctggcctcacacgttcggaggggggaccaaggtggaaattaag

SEQ ID NO: 4 - protein, 107 aa, Mus sp.
EIVLTQSPATLSLSPGERATLSCQASESISNHLHWYQQRPGQAPRLLIKYRSQSISGIPARFSGSGSGTDFTLTISSLEPEDFAVYYCQQSGSWPHTFGGGTKVEIK

SEQ ID NO: 5 - protein, 5 aa, Mus sp.
SYDMS

SEQ ID NO: 6 - protein, 7 aa, Mus sp.
KVSSGGG

SEQ ID NO: 7 - protein, 8 aa, Mus sp.
HNYGSFAY

SEQ ID NO: 8 - protein, 11 aa, Mus sp.
QASQSISNHLH

SEQ ID NO: 9 - protein, 7 aa, Mus sp.
YRSQSIS

SEQ ID NO: 10 - protein, 9 aa, Mus sp.
QQSGSWPHT

SEQ ID NO: 11 - protein, 39 aa, Homo sapiens
YMYISPPEALENPCYDMKTTCLPMFGYKHVLTLTDQVTR

SEQ ID NO: 12 - protein, 39 aa, Cricetulus sp.
YMFISPPEALKNPCYSMKTSCLPMFGYKHVLTLTDQVTR

SEQ ID NO: 13 - protein, 39 aa, Lepus sp.
YMYISPPEALRNPCYDMKTTCLPMFGYKHVLTLTDQVTR

SEQ ID NO: 14 - protein, 39 aa, Gallus sp.
YMYISPPEAIKNPCYEIGEKCLPMFGYKHVLTLTDEVMR

SEQ ID NO: 15 - protein, 39 aa, Rattus sp.
YMFISPPQAIKNPCYTMKSTCLPMFGYKHVLTLTDQVTR

SEQ ID NO: 16 - protein, 39 aa, Mus sp.
YMYISPPQAIKNPCYNMKNACLPMFGYKHVLTLTDQVTR

(19) United States
(12) Patent Application Publication
(10) Pub. No.: US 2009/0148459 A1
(43) Pub. Date: Jun. 11, 2009

(54) Prevention or Treatment of Cancer Using Integrin alphavbeta3 Antagonists in Combination with Other Agents

(76) Inventors: Richard Woessner, Lafayette, CO (US); Peter Kiener, Doylestown, PA (US); Melissa Dormitzer, Germantown, MD (US); William Walsh, Sharpsburg, MD (US); Jon Heinrichs, North Potomac, MD (US)

Correspondence Address: MEDIMMUNE, LLC;Jonathan Klein-Evans, ONE MEDIMMUNE WAY, GAITHERSBURG, MD, 20878, US

(21) Appl. No.: 11/971,675

(22) Filed: Jan. 9, 2008

Related U.S. Application Data
(63) Continuation of application No. 10/379,189, filed on Mar. 4, 2003.
(60) Provisional application No. 60/361,859, filed on Mar. 4, 2002.
(60) Provisional application No. 60/370,398, filed on Apr. 5, 2002.
(60) Provisional application No. 60/444,265, filed on Jan. 30, 2003.

(51) Int. Cl. A61K 39/395; A61P 35/00

(52) U.S. Cl. 424/145.1; 424/173.1

(57) ABSTRACT

The present invention relates to methods and compositions designed for the treatment, management or prevention of cancer. The methods of the invention comprise the administration of an effective amount of one or more antagonists of Integrin α_{V}β₃ alone or in combination with the administration of an effective amount of one or more other agents useful for cancer therapy. The invention also provides pharmaceutical compositions comprising one or more antagonists of Integrin α_{V}β₃ and/or one or more other agents useful for cancer therapy. In particular, the invention is directed to methods of treatment and prevention of cancer by the administration of a therapeutically or prophylactically effective amount of one or more antagonists of Integrin α_{V}β₃ alone or in combination with standard and experimental therapies for treatment or prevention of cancer. Also included are methods for screening for epitope-specific Integrin α_{V}β₃ antagonists which can be used according to the methods of the invention. In addition, methods for facilitating the use of Integrin α_{V}β₃ antagonists in the analysis of Integrin α_{V}β₃ expression in biopsies of animal model and clinical study samples are also contemplated.

This application is entitled to and claims priority benefits to U.S. Provisional Application Ser. No. 60/361,859, filed Mar. 4, 2002, U.S. Provisional Application Ser. No. 60/370,398, filed Apr. 5, 2002, and U.S. Provisional Application Ser. No. 60/444,265, filed Jan. 30, 2003, each of which is incorporated herein by reference in its entirety.

### 1. FIELD OF THE INVENTION

The invention relates to therapeutic regimens or protocols designed for the prevention, management, treatment or amelioration of cancer or one or more symptoms thereof. Such protocols involve the administration of a prophylactically or therapeutically effective amount of one or more antagonists of Integrin αᵥβ₃ alone or in combination with the administration of a prophylactically or therapeutically effective amount of one or more other therapies useful for cancer therapy. In particular, the invention provides methods for preventing, managing, treating or ameliorating cancer or one or more symptoms thereof, said methods comprising administering to a subject in need thereof a dose of a prophylactically or therapeutically effective amount of one or more antagonists of Integrin αᵥβ₃ alone or in combination with the administration of a standard or experimental chemotherapy, a hormonal therapy, a biological therapy/immunotherapy and/or a radiation therapy. The invention also provides methods for preventing, managing, treating or ameliorating cancer or one or more symptoms thereof, said methods comprising administering to a subject in need thereof a dose of a prophylactically or therapeutically effective amount of one or more antagonists of Integrin αᵥβ₃ in combination with surgery, alone or in further combination with the administration of a standard or experimental chemotherapy, a hormonal therapy, a biological therapy/immunotherapy and/or a radiation therapy. The antagonists of Integrin αᵥβ₃ utilized to prevent, manage, treat or ameliorate cancer or one or more symptoms thereof may or may not be conjugated or fused to a moiety (e.g., a therapeutic agent or drug). The methods of the invention are particularly useful for the prevention, management, treatment or amelioration of breast cancer, colon cancer, prostate cancer, melanoma, lung cancer, glioblastoma, ovarian cancer, and cancers that have the potential to metastasize or have metastasized to other organs or tissues, in particular, bone. The invention also provides methods for screening for epitope-specific Integrin αᵥβ₃ antagonists which can be used in accordance with the methods of the invention. Further, the invention provides pharmaceutical compositions and kits for use in preventing, managing, treating or ameliorating cancer or one or more symptoms thereof.

### 2. BACKGROUND OF THE INVENTION

### Cancer

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading case of death in the United States and if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

A cure for cancer has yet to be found. Current treatment options, such as surgery, chemotherapy and radiation treatment, are oftentimes either ineffective or present serious side effects.

### Cancer Therapy

Currently, cancer therapy may involve surgery, chemotherapy, hormonal therapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although can be effective, is often used to prevent or delay recurrence of cancer after other treatments have removed the majority of the cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (see, for example, Gilman et al., Goodman and Gilman's: The Pharmacological Basis of Therapeutics, Eighth Ed. (Pergamom Press, New York, 1990)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic agents for the treatment of cancer and new, more effective, therapy combinations for the treatment of cancer.

### 3. SUMMARY OF THE INVENTION

The present invention encompasses treatment protocols that provide better prophylactic or therapeutic profiles than current single agent therapies or combination therapies for cancer. In particular, the invention encompasses the use of an antagonist of Integrin αᵥβ₃ for the prevention, management, treatment or amelioration of cancer or one or more symptoms thereof. The invention also encompasses treatment protocols that enhance the prophylactic or therapeutic effect of an antagonist of Integrin αᵥβ₃ (preferably, an antibody that immunospecifically binds to Integrin αᵥβ₃). The invention also encompasses the use of an antagonist of Integrin αᵥβ₃ (preferably, an antibody that immunospecifically binds to Integrin αᵥβ₃) conjugated or fused to a moiety (e.g., therapeutic agent or drug) for preventing, managing, treating or ameliorating cancer or one or more symptoms thereof.

The invention provides methods for preventing, managing, treating or ameliorating cancer that has the potential to metastasize or has metastasized to an organ or tissue (e.g., bone) or one or more symptoms thereof, said methods comprising administering to a subject in need thereof one or more doses of a prophylactically or therapeutically amount of an antagonist of Integrin αᵥβ₃ (preferably, an antibody that immunospecifically binds to Integrin αᵥβ₃). In a specific embodiment, the invention provides methods for preventing, managing, treating or ameliorating cancer that has the potential to metastasize or has metastasized to the bone or one or more symptoms thereof, said methods comprising administering to a subject in need thereof one or more doses of a prophylactically or therapeutically effective amount of one or more antagonists of Integrin αᵥβ₃. In a preferred embodiment, the invention provides methods for preventing, managing, treating or ameliorating prostate cancer that has the potential to metastasize or has metastasized to the bone or one or more symptoms thereof, said methods comprising administering to a subject in need thereof one or more doses of a prophylactically or therapeutically effective amount of one or more antibodies or fragments thereof that immunospecifically bind to Integrin αᵥβ₃.

The invention provides methods for preventing, managing, treating or ameliorating cancer or one or more symptoms thereof, said methods comprising administering to a subject in need thereof one or more doses of a prophylactically or therapeutically effective amount of an antagonist of Integrin αᵥβ₃ (preferably, an antibody that immunospecifically binds to Integrin αᵥβ₃, and more preferably, VITAXIN® or an antigen-binding fragment thereof) fused or conjugated to a moiety (e.g., a therapeutic agent or drug). In a specific embodiment, the invention provides methods for preventing, managing, treating or ameliorating cancer that has the potential to metastasize or has metastasized to an organ or tissue (e.g., bone) or one or more symptoms thereof, said methods comprising administering to a subject in need thereof one or more doses of a prophylactically or therapeutically effective amount of an antagonist of Integrin αᵥβ₃ fused or conjugated to a moiety (e.g., a therapeutic agent or drug). In a particular embodiment, the invention provides methods for preventing, managing, treating or ameliorating cancer that has the potential to metastasize or has metastasized to the bone or one or more symptoms thereof, said methods comprising administering to a subject in need thereof one or more doses of a prophylactically or therapeutically effective amount of one or more antibodies or fragments thereof that immunospecifically bind to Integrin αᵥβ₃ fused or conjugated to a moiety (e.g., a therapeutic agent or drug). In another preferred embodiment, the invention provides methods for preventing, managing, treating or ameliorating cancer that has the potential to metastasize or has metastasized to the bone or one or more symptoms thereof, said methods comprising administering to a subject in need thereof one or more doses of a prophylactically or therapeutically effective amount of VITAXIN® or an antigen-binding fragment thereof fused or conjugated to a moiety (e.g., a therapeutic agent or drug). Examples of a moiety that an antagonist of Integrin αᵥβ₃ (e.g., an anti-Integrin αᵥβ₃ antibody or a fragment thereof) can be fused or conjugated to include, but are not limited to, those agents disclosed in Section 5.5.1 infra.

The present invention encompasses protocols for the prevention, management, treatment or amelioration of cancer or one or more symptoms thereof in which an antagonist of Integrin αᵥβ₃ is used in combination with a therapy (e.g., prophylactic or therapeutic agent) other than an antagonist of Integrin αᵥβ₃. The invention is based, in part, on the recognition that antagonists of Integrin αᵥβ₃ potentiate and synergize with, enhance the effectiveness of, improve the tolerance of, and/or reduce the side effects caused by, other cancer therapies, including current standard and experimental chemotherapies. The combination therapies of the invention have additive potency, an additive therapeutic effect or a synergistic effect. The combination therapies of the invention enable lower dosages of the therapy (e.g., prophylactic or therapeutic agents) utilized in conjunction with antagonists of Integrin αᵥβ₃ for the prevention, management, treatment or amelioration of cancer and/or less frequent administration of such prophylactic or therapeutic agents to a subject with cancer to improve the quality of life of said subject and/or to achieve a prophylactic or therapeutic effect. The combination therapies of the invention enable lower dosages of one or more antagonists of Integrin αᵥβ₃ and/or less frequent administration of dosages of one or more antagonists of Integrin αᵥβ₃ to a subject with cancer to improve the quality of life of said subject and/or to achieve a prophylactic or therapeutic effect. Further, the combination therapies of the invention reduce or avoid unwanted or adverse side effects associated with the administration of current single agent therapies and/or existing combination therapies for cancer, which in turn improves patient compliance with the treatment protocol.

The present invention provides methods for preventing, managing, treating or ameliorating cancer or one or more symptoms thereof, said methods comprising administering to a subject in need thereof a dosage of a prophylactically or therapeutically effective amount of one or more antagonists of Integrin αᵥβ₃ in combination with the administration of a dosage of a prophylactically or therapeutically effective amount of one or more other therapies useful for the prevention, treatment, management or amelioration of cancer, or a symptom thereof. Examples of cancer therapies that can be used in combination with one or more antagonists of Integrin α_{V}β₃ include, but are not limited to those disclosed in Section 5.6 infra. In one embodiment, an antagonist of Integrin α_{V}β₃ is administered to a subject in need thereof in combination with another cancer therapy that works by the same mechanism as the antagonist of Integrin α_{V}β₃. In another embodiment, an antagonist of Integrin α_{V}β₃ is administered to a subject in need thereof in combination with another cancer therapy that works by a different mechanism than the antagonist of Integrin α_{V}β₃. By example and not by limitation, the cancer therapy can be apoptosis inducing, cytotoxic, antimitotic, tubulin stabilizing, microtubule formation inhibiting, topoisomerase active, antimetabolic, or DNA interactive agents. In other embodiments, the cancer therapy administered with an antagonist of Integrin α_{V}β₃ is gene therapy based. In other embodiments, the therapy is another antibody that is not an antagonist of Integrin α_{V}β₃.

In one embodiment, the invention provides methods for preventing, managing, treating or ameliorating cancer or one or more symptoms thereof, said methods comprising administering to a subject in need thereof a dose of a prophylactically or therapeutically effective amount of one or more antagonists of Integrin αᵥβ₃ in combination with the administration of a standard or experimental chemotherapy, a hormonal therapy, a biological therapy/immunotherapy and/or a radiation therapy. In another embodiment, the invention provides methods for preventing, managing, treating or ameliorating cancer or one or more symptoms thereof, said methods comprising administering to a subject in need thereof a dose of a prophylactically or therapeutically effective amount of one or more antagonists of Integrin αᵥβ₃ in combination with surgery, alone or in further combination with the administration of a standard or experimental chemotherapy, a hormonal therapy, a biological therapy/immunotherapy and/or a radiation therapy. In accordance with these embodiments, the antagonists of Integrin αᵥβ₃ utilized to prevent, manage, treat or ameliorate cancer or one or more symptoms thereof may or may not be conjugated or fused to a moiety (e.g., therapeutic agent or drug) and such antagonists are preferably antibodies that immunospecifically bind to Integrin α_{V}β₃, more preferably Vitaxin® or an antigen-binding fragment thereof.

The invention provides methods for preventing, managing, treating or ameliorating cancer or one or more symptoms thereof, said methods comprising administering to a subject in need thereof one or more antagonists of Integrin αᵥβ₃ (preferably, antibodies that immunospecifically bind to Integrin αᵥβ₃) in combination with one or more therapeutic agents that are not cancer therapeutics (a.k.a., non-cancer therapies). Examples of such agents include, but are not limited to, anti-emetic agents, anti-fungal agents, anti-bacterial agents, such as antibiotics, anti-inflammatory agents, and anti-viral agents. Non-limiting examples of anti-emetic agents include metopimazin and metochlopramide. Non-limiting examples of antifungal agents include azole drugs, imidazole, triazoles, polyene, amphottericin and ryrimidine. Non-limiting examples of anti-bacterial agents include dactinomycin, bleomycin, erythromycin, penicillin, mithramycin, cephalosporin, imipenem, axtreonam, vancomycin, cycloserine, bacitracin, chloramphenicol, clindamycin, tetracycline, streptomycin, tobramycin, gentamicin, amikacin, kanamycin, neomycin, spectinomycin, trimethoprim, norfloxacin, refampin, polymyxin, amphotericin B, nystatin, ketocanazole, isoniazid, metronidazole and pentamidine. Non-limiting examples of antiviral agents include nucleoside analogs (e.g., zidovudine, acyclivir, gangcyclivir, vidarbine, idoxuridine, trifluridine and ribavirin), foscaret, amantadine, rimantadine, saquinavir, indinavir, ritonavir, interferon (“IFN”)-α, β or γ and AZT. Non-limiting examples of anti-inflammatory agents include non-steroidal anti-inflammatory drugs (“NSAJDs”), steroidal anti-inflammatory drugs, beta-agonists, anti-cholingenic agents and methylxanthines.

In a specific embodiment, the invention provides methods for preventing, managing, treating or ameliorating cancer or one or more symptoms thereof, said methods comprising administering a dose of a prophylactically or therapeutically effective amount of one or more antagonists of Integrin αᵥβ₃ in combination with a dose of a prophylactically or therapeutically effective amount of one or more therapeutic agents that are not cancer therapeutics. In another embodiment, the invention provides methods for preventing, managing, treating or ameliorating cancer or one or more symptoms thereof, said methods comprising administering to a subject in need thereof a dose of a prophylactically or therapeutically effective amount of one or more antagonists of Integrin αᵥβ₃ in combination with the administration of a dose of a prophylactically or therapeutically effective amount of one or more therapeutic agents that are not cancer therapeutics, and the administration of a standard or experimental chemotherapy, a hormonal therapy, a biological therapy/immunotherapy and/or a radiation therapy. In accordance with these embodiments, the subject may undergo or have undergone surgery and the antagonists of Integrin αᵥβ₃ utilized to prevent, manage, treat or ameliorate cancer or one or more symptoms thereof may or may not be conjugated or fused to a moiety (e.g., therapeutic agent or drug).

In one embodiment, the invention provides methods for preventing, managing, treating or ameliorating cancer or one or more symptoms thereof, said methods comprising administrating to a subject in need thereof a dose of a prophylactically or therapeutically effective amount of an antagonist of Integrin α_{V}β₃ in combination with one or more doses of a prophylactically or therapeutically effective amount of one or more chemotherapies alone or, optionally, in combination with one or more doses of a prophylactically or therapeutically effective amount of hormonal therapies, biological therapies/immunotherapies and/or radiation other than Integrin αᵥβ₃ antagonists. In another embodiment, the invention provides methods for preventing, managing, treating or ameliorating cancer or one or more symptoms thereof, said methods comprising administrating to a subject in need thereof a dose of a prophylactically or therapeutically effective amount of an antagonist of Integrin α_{V}β₃ in combination with one or more doses of a prophylactically or therapeutically effective amount of one or more hormonal therapies alone or, optionally, in combination with one or more doses of a prophylactically or therapeutically effective amount of chemotherapies, biological therapies/immunotherapies and/or radiation therapies other than Integrin α_{V}β₃ antagonists. In another embodiment, the invention provides methods for preventing, managing, treating or ameliorating cancer or one or more symptoms thereof, said methods comprising administrating to a subject in need thereof a dose of a prophylactically or therapeutically effective amount of an antagonist of Integrin α_{V}β₃ in combination with one or more doses of a prophylactically or therapeutically effective amount of one or more biological therapies/immunotherapies alone or, optionally, in combination with one or more doses of a prophylactically or therapeutically effective amount of chemotherapies, hormonal therapies and/or radiation therapies other than Integrin α_{V}β₃ antagonists. In yet another embodiment, the invention provides methods for preventing, managing, treating or ameliorating cancer or one or more symptoms thereof, said methods comprising administrating to a subject in need thereof a dose of a prophylactically or therapeutically effective amount of an antagonist of Integrin α_{V}β₃ in combination with one or more doses of a prophylactically or therapeutically effective amount of one or more radiation therapies alone or, optionally, in combination with one or more doses of a prophylactically or therapeutically effective amount of chemotherapies, hormonal therapies, and/or biological therapies/immunotherapies other than Integrin α_{V}β₃ antagonists. In accordance with these embodiments, the subject may undergo or have undergone surgery and the antagonists of Integrin αᵥβ₃ utilized to prevent, manage, treat or ameliorate cancer or one or more symptoms thereof may or may not be conjugated or fused to a moiety (e.g., a therapeutic agent or drug).

The present invention provides methods for preventing, managing, treating or ameliorating cancer or one or more symptoms thereof, said methods comprising administrating to a subject in need thereof an antagonist of Integrin α_{V}β₃ in combination with surgery. In a specific embodiment, the invention provides methods for preventing, managing, treating or ameliorating cancer or one or more symptoms thereof, said methods comprising administrating to a subject in need thereof a dose of a prophylactically or therapeutically effective amount of one or more antagonists of Integrin α_{V}β₃ in combination with surgery. In another embodiment, the invention provides methods for preventing, managing, treating or ameliorating cancer or one or more symptoms thereof, said methods comprising administrating to a subject in need thereof a dose of a prophylactically or therapeutically effective amount of one or more antagonists of Integrin α_{V}β₃ that is conjugated or fused to a moiety (e.g., therapeutic agent or drug) in combination with surgery. In accordance with these embodiments, the Integrin α_{V}β₃ antagonists are preferably antibodies that immunospecifically bind to Integrin α_{V}β₃ and more preferably, Vitaxin® or an antigen-binding fragment thereof.

The invention provides methods for preventing, managing, treating or ameliorating cancer or one or more symptoms thereof, said method comprising: (a) administering to a subject in need thereof a dose of a prophylactically or therapeutically effective amount of one or more Integrin α_{V}β₃ antagonists (preferably, one or more antibodies or fragments thereof that immunospecifically bind to Integrin α_{V}β₃) and a dose of a prophylactically or therapeutically effective amount of one or more other anti-cancer therapies; and (b) administering one or more subsequent doses of said Integrin α_{V}β₃ antagonists, to maintain a plasma concentration of the antagonist at a desirable level (e.g., about 0.1 to about 100 μg/ml), which continuously blocks the Integrin α_{V}β₃ activity. In a specific embodiment, the plasma concentration of the antagonist is maintained at 10 μg/ml, 15 μg/ml, 20 μg/ml, 25 μg/ml, 30 μg/ml, 35 μg/ml, 40 μg/ml, 45 μg/ml or 50 μg/ml.

In a specific embodiment, the invention provides methods for preventing, managing, treating or ameliorating cancer or one or more symptoms thereof, said methods comprising administrating to a subject in need thereof one or more doses of a prophylactically or therapeutically effective amount of an antagonist of Integrin α_{V}β₃, preferably Vitaxin® or an antigen-binding fragment thereof, in combination with one or more doses of a prophylactically or therapeutically effective amount of one or more cancer chemotherapeutic agents, such as but not limited to: doxorubicin, epirubicin, cyclophosphamide, 5-fluorouracil, taxanes such as docetaxel and paclitaxel, leucovorin, levamisole, irinotecan, estramustine, etoposide, vinblastine, dacarbazine, nitrosoureas such as carmustine and lomustine, vinca alkaloids, platinum compounds, cisplatin, mitomycin, vinorelbine, gemcitabine, carboplatin, hexamethylmelamine and/or topotecan. Such methods can optionally further comprise the administration of one or more doses of prophylactically or therapeutically effective amount of other cancer therapies, such as but not limited to radiation therapy, biological therapies, hormonal therapies and/or surgery. In another embodiment, the invention provides methods for preventing, managing, treating or ameliorating cancer or one or more symptoms thereof, said methods comprising administrating to a subject in need thereof one or more doses of prophylactically or therapeutically effective amount of an antagonist of Integrin α_{V}β₃, preferably Vitaxin® or an antigen-binding fragment thereof, in combination with administration of one or more doses of prophylactically or therapeutically effective amount of one or more cancer therapeutic agents, wherein the cancer therapeutic agents are not cancer chemotherapeutic agents.

In a specific embodiment, the invention provides methods for preventing, managing, treating or ameliorating cancer or one or more symptoms thereof, said methods comprising administrating to a subject in need thereof an antagonist of Integrin α_{V}β₃, preferably Vitaxin®b or an antigen-binding fragment thereof, in combination with administration of one or more immunomodulatory agents, including but not limited to, cytokines and antibodies. In a preferred embodiment, the immunomodulatory agents are immunosuppressant agents. In certain embodiments, the immunomodulatory agents are cancer chemotherapeutic agents. In other embodiments, the immunomodulatory agents are agents other than chemotherapeutic agents. In yet other embodiments, the immunomodulatory agents are agents other than interleukins or hemopoietic factors such as IL-1, IL-4, IL-6, IL-12, IL-15, TNF, IFN-α, IFN-β, IFN-γ, M-CSF, G-CSF, IL-3 and erythropoietin.

In another specific embodiment, the invention provides methods for preventing, managing, treating or ameliorating cancer or one or more symptoms thereof, said methods comprising administrating to a subject in need thereof one or more doses of a prophylactically or therapeutically effective amount of an antagonist of Integrin α_{V}β₃, preferably Vitaxin® or an antigen-binding fragment thereof, in combination with administration of one or more doses of a prophylactically or therapeutically effective amount of one or more types of radiation therapy, such as external-beam radiation therapy, interstitial implantation of radioisotopes (1-125, palladium, and iridium), radioisotopes such as strontium-89, thoracic radiation therapy, intraperitoneal P-32 radiation therapy, and/or total abdominal and pelvic radiation therapy. Such methods can optionally further comprise the administration of one or more doses of a prophylactically or therapeutically effective amount of other cancer therapies, such as but not limited to chemotherapies, biological therapies/immunotherapies, hormonal therapies and/or surgery.

In a specific embodiment, the invention provides methods for preventing, managing, treating or ameliorating cancer or one or more symptoms thereof, said methods comprising administrating to a subject in need thereof one or more doses of a prophylactically or therapeutically effective amount of an antagonist of Integrin α_{V}β₃ in combination with one or more doses of a prophylactically or therapeutically effective amount of one or more biological therapies/immunotherapies or hormonal therapies other than Integrin α_{V}β₃ antagonists. Such methods can optionally further comprise the administration of one or more doses of a prophylactically or therapeutically effective amount of other cancer therapies, such as but not limited to radiation therapy, chemotherapies, and/or surgery. Examples of such biological therapies/immunotherapies include, but are not limited to, tamoxifen, leuprolide or other LHRH agonists, non-steroidal antiandrogens (flutamide, nilutamide, bicalutamide), steroidal antiandrogens (cyproterone acetate), estrogens (DES, chlorotrianisene, ethinyl estradiol, conjugated estrogens U.S.P., DES-diphosphate), aminoglutethimide, hydrocortisone, flutamide withdrawal, progesterone, ketoconazole, prednisone, interferon-alpha, interferon-beta, interferon-gamma, interleukin-2, tumor necrosis factor-alpha, and melphalan. Biological therapies also include cytokines such as, but not limited to, TNF ligand family members such as TRAIL anti-cancer agonists that induce apoptosis, TRAIL antibodies that bind to TRAIL receptors 1 and 2 otherwise known as DR4 and DR5 (Death Domain Containing Receptors 4 and 5), as well as DR4 and DR5. TRAIL and TRAIL antibodies, ligands and receptors are known in the art and described in U.S. Pat. Nos. 6,342,363, 6,284,236, 6,072,047 and 5,763,223.

In one embodiment, the antagonist of Integrin α_{V}β₃ used in accordance with the methods of the invention is an antibody or a fragment thereof that immunospecifically binds to Integrin α_{V}β₃. In a preferred embodiment, the antagonist of Integrin α_{V}β₃ used in accordance with the methods of the invention is an LM609 antibody or an antibody derived therefrom that immunospecifically Integrin α_{V}β₃, such as chimerized and humanized versions of LM609, for example the antibody Vitaxin®. Such antibodies have been described in International Publication Nos. WO 89/05155, WO 98/33919 and WO 00/78815 as well as U.S. Pat. No. 5,753,230, which are incorporated by reference herein in their entireties. In a particular embodiment, the antagonist of Integrin α_{V}β₃ used in accordance with the methods of the invention is an antibody or fragment thereof that competes with LM609 or Vitaxin®, or an antigen-binding fragment thereof for binding to Integrin α_{V}β₃. In accordance with this embodiment, the antibody or fragment thereof that competes with LM609 or Vitaxin® or an antigen-binding fragment thereof for binding to Integrin α_{V}β₃ preferably does not include the monoclonal antibody D12 or an antigen-binding fragment thereof disclosed in International Publication No. WO 98/40488.

In other embodiments, the invention provides antibodies that immunoreact with Ecr, the RGD-directed adhesion receptor found on the surface of both endothelial and melanoma cells. Encompassed by the invention are antibodies which are useful for inhibiting the ability of cells that contain the adhesion receptor to adhere to a subendothelial matrix composed of vitronectin, fibrinogen or von Willegrand factor. Also encompassed by the invention are antibodies that inhibit functional activity of Integrin α_{V}β₃ or inhibit Integrin α_{V}β₃-mediated pathologies. Accordingly, the invention provides antibodies useful for the inhibition of angiogenesis or the inhibition of other functions mediated or influenced by Integrin α_{V}β₃, including but not limited to cell proliferation, cell attachment, cell migration, granulation tissue development, and/or inflammation. Such antibodies have been described in International Publication Nos. WO 89/05155, WO 98/33919 and WO 00/78815 as well as U.S. Pat. No. 5,753,230, which are incorporated by reference herein in their entireties.

The invention provides protocols for the administration of an antagonist of Integrin α_{V}β₃ alone or in combination with other cancer or non-cancer therapies to a subject in need thereof. The therapies (e.g., prophylactical or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve the efficacy of the therapies.

The therapies (e.g., prophylactic or therapeutic agents) of the combination therapies of the invention can be administered to a subject concurrently. The term “concurrently” is not limited to the administration of therapies (e.g., prophylactic or therapeutic agents) at exactly the same time, but rather it is meant that an antagonist of Integrin α_{V}β₃ and another therapy(ies) are administered to a subject in a sequence and within a time interval such that the Integrin α_{V}β₃ can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In preferred embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The present invention encompasses pharmaceutical compositions comprising one or more antagonists of Integrin α_{V}β₃ and a pharmaceutically acceptable carrier. The present invention also encompasses pharmaceutical compositions comprising one or more antagonists of Integrin α_{V}β₃ conjugated or fused to a moiety (e.g., a therapeutic agent or drug), and a pharmaceutically acceptable carrier. The present invention encompasses the use of pharmaceutical compositions comprising one or more prophylactic or therapeutic agents other than Integrin α_{V}β₃ antagonists and a pharmaceutically acceptable carrier. The present invention provides pharmaceutical compositions comprising one or more antagonists of Integrin α_{V}β₃, one or more prophylactic or therapeutic agents useful for the prevention, management, treatment or amelioration of cancer or one or more symptoms thereof other than antagonists of Integrin α_{V}β₃, and a pharmaceutically acceptable carrier. The present invention further provides pharmaceutical compositions comprising one or more antagonists of Integrin α_{V}β₃ conjugated or fused to a moiety (e.g., a therapeutic agent or drug), one or more prophylactic or therapeutic agents useful for the prevention, management, treatment or amelioration of cancer or one or more symptoms thereof other than antagonists of Integrin α_{V}β₃, and a pharmaceutically acceptable carrier.

The pharmaceutical compositions of the invention may be used in accordance with the methods of the invention for the prevention, management, treatment or amelioration of cancer or one or more symptoms thereof. Preferably, the pharmaceutical compositions of the invention are sterile and in suitable form for a particular method of administration to a subject with cancer.

The methods and compositions of the invention are useful in preventing, managing, treating or ameliorating cancers, including, but not limited to, the cancers disclosed in Section 5.1.1.1 infra. Specific examples of cancers that can be prevented, managed, treated or ameliorated in accordance with the invention include, but are not limited to, cancer of the head, neck, eye, mouth, throat, esophagus, chest, bone, lung, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. In a specific embodiment, the methods and compositions of the invention are used for the prevention, management, treatment or amelioration of a primary or secondary cancer that expresses Integrin α_{V}β₃. In another embodiment, the methods and compositions of the invention are used for the prevention, management, treatment or amelioration of a primary or secondary cancer that does not express Integrin α_{V}β₃. In a preferred embodiment, the methods and compositions are used for the prevention, management, treatment or amelioration of a cancer that has the potential to metastasize or has metastasized to other tissues or organs (e.g., bone). In another preferred embodiment, the methods and compositions of the invention are used for the prevention, management, treatment or amelioration of lung cancer, prostate cancer, ovarian cancer, melanoma, bone cancer or breast cancer.

The methods and compositions of the invention are useful not only in untreated cancer patients but are also useful in the management or treatment of cancer patients partially or completely refractory to current standard and experimental cancer therapies, including, but not limited to, chemotherapies, hormonal therapies, biological therapies, radiation therapies, and/or surgery. In a specific embodiment, the methods and compositions of the invention are useful for the prevention, management, treatment or amelioration of cancer that has been shown to be or may be refractory or non-responsive to therapies other than those comprising the administration of Integrin α_{V}β₃ antagonists. In a preferred embodiment, the methods and compositions of the invention are useful for the prevention, management, treatment or amelioration of cancer that has been shown to be or may be refractory or non-responsive to therapies comprising administration of an antibody or fragment thereof that immunospecifically binds to Integrin α_{V}β₃, preferably Vitaxin® or an antigen-binding fragment thereof. The methods and compositions of the invention are also useful for the prevention, management, treatment or amelioration of cancer or one or more symptoms thereof in patients that do not tolerate therapies other than antagonists for Integrin α_{V}β₃ (preferably antibodies or fragments thereof that immuno-specifically bind to Integrin α_{V}β₃ and more preferably Vitaxin® or an antigen-binding fragment thereof) because of unwanted or adverse effects.

The invention also provides methods for screening for antagonists for Integrin α_{V}β₃. In certain embodiments, amino acid substitutions are made in the subunits of Integrin α_{V}β₃, for example to change the ligand specificity of the Integrin α_{V}β₃ and/or disrupt the heterodimerization of the subunit chains. In specific embodiments, such amino acid substitutions disrupt the specific interaction of certain antagonists of Integrin α_{V}β₃ with a particular Integrin α_{V}β₃ epitope. In a preferred embodiment, the amino acid substitutions are made within regions of an Integrin subunit that confer ligand binding specificity, preferably ligand binding specificity of LM609 and/or Vitaxin®. In a specific preferred embodiment, amino acids 171, 173 and 174 of the human β₃ subunit can be substituted, preferably with Gln, Ile and Lys, respectively, to disrupt binding to Vitaxin®. In another preferred embodiment, the amino acid substitutions are made in the β₃ subunit, preferably with Gln, Ile, Lys, Thr and Ser, at amino acids 171, 173, 174, 179, and 182, respectively. Accordingly, such amino acid substituted subunits of Integrin α_{V}β₃ can be used for screening antibodies with specific affinity for particular epitopes by identifying monoclonal antibodies that bind to wild type Integrin α_{V}β₃ but not the mutant form. In other embodiments, methods of the invention involve screening for antagonists that bind the region of amino acids 164-202 of human β₃ chain in the context of the heterodimer. In addition, the invention provides methods for identifying monoclonal antibodies that bind to the heterodimerized α_{V}β₃ but not the α_{V} or the β₃ chains when not included in a heterodimer. The antibodies identified utilizing such screening methods can be used for the prevention, treatment, management or amelioration of Integrin α_{V}β₃-mediated diseases and disorders or one or more symptoms thereof, including but not limited to cancer, inflammatory and autoimmune diseases either alone or in combination with other therapies. Preferably, these antibodies are not LM609, VITAXIN®, D12 or an antibody or antibody binding fragment thereof having the CDRs of LM609, VITAXIN® or D12 with no more than one, no more than two, no more than five, no more than eight, or no more than ten amino acid substitutions, deletions or insertions.

The invention provides methods of detecting, diagnosing and/or monitoring the progression of cancer utilizing one or more antagonists Integrin α_{V}β₃ (preferably, one or more antibodies that immunospecifically bind to Integrin α_{V}β₃ and more preferably Vitaxin® or an antigen-binding fragment thereof) conjugated or fused to a detectable agent. In particular, methods for facilitating the use of Integrin α_{V}β₃ antagonists in the analysis of Integrin α_{V}β₃ expression in biopsies of animal model and clinical study samples are also provided.

The present invention provides kits comprising one or more antagonists Integrin α_{V}β₃ (preferably, one or more antibodies that immunospecifically bind to Integrin α_{V}β₃ and more preferably Vitaxin® or an antigen-binding fragment thereof) conjugated or fused to a detectable agent, therapeutic agent or drug, in one or more containers, for use in the prevention, treatment, management, amelioration, detection, monitoring or diagnosis of cancer. The invention also provides kits comprising one or more antagonists Integrin α_{V}β₃ (preferably, one or more antibodies that immunospecifically bind to Integrin α_{V}β₃ and more preferably Vitaxin® or an antigen-binding fragment thereof) in a first vial and one or more prophylactic or therapeutic agents, other than antagonists of Integrin α_{V}β₃, in a second vial for use in the prevention, treatment, management, amelioration, detection, monitoring or diagnosis of cancer. The invention also provides kits comprising one or more antagonists Integrin α_{V}β₃ (preferably, one or more antibodies that immunospecifically bind to Integrin α_{V}β₃ and more preferably Vitaxin® or an antigen-binding fragment thereof) conjugated or fused to a therapeutic agent or drug in a first vial and one or more prophylactic or therapeutic agents, other than antagonists of Integrin α_{V}β₃, in a second vial for use in the prevention, treatment, management, amelioration, detection, monitoring or diagnosis of cancer. The kits may further comprise packaging materials and/or instructions.

The present invention also provides articles of manufacture.

3.1 Terminology

As used herein, the term “adjunctive” is used interchangeably with “in combination” or “combinatorial.” Such terms are also used where two or more therapeutic or prophylactic agents affect the treatment or prevention of the same disease.

As used herein, the term “analog” in the context of proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, peptidomimetic, glycoprotein, antibody, antibody fragment, carbohydrate, nucleic acid, organic molecule, inorganic molecule, large molecule, or small molecule that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “antibody” and “antibodies” refer to monoclonal antibodies, multispecific antibodies, human antibodies, humanized antibodies, camelised antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the terms “anti-Integrin αᵥβ₃ antibodies” and “Integrin α_{V}β₃ antibodies” refer to the antibodies described in Section 5.4 infra.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the terms “disorder” and “disease” are used interchangeably to refer to a condition in a subject. Certain conditions may be characterized as more than one disorder.

As used herein, the term “effective amount” refers to the amount of a therapy (e.g., a prophylactic or therapeutic agent) which is sufficient to reduce or ameliorate the severity, duration and/or progression of cancer or one or more symptoms thereof, ameliorate one or more symptoms of cancer, prevent the advancement of cancer, cause regression of cancer, prevent the recurrence, development, or onset of cancer or one or more symptoms thereof, or enhance or improve the prophylactic or therapeutic effect(s) of another therapy (e.g., prophylactic or therapeutic agent).

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays (see Section 5.2.1.2 infra). Antigenic epitopes need not necessarily be immunogenic.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or protein. In a specific embodiment, a fragment of a protein or polypeptide retains at least one function of the protein or polypeptide. In another embodiment, a fragment of a protein or polypeptide retains at least two, three, four, or five functions of the protein or polypeptide. Preferably, a fragment of an antibody retains the ability to immunospecifically bind to Integrin αᵥβ₃.

As used herein, the term “fusion protein” refers to a polypeptide that comprises an amino acid sequence of a first protein or polypeptide or functional fragment, analog or derivative thereof, and an amino acid sequence of a heterologous protein, polypeptide, or peptide (i.e., a second protein or polypeptide or fragment, analog or derivative thereof different than the first protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides or peptides with immunomodulatory activity may be fused together to form a fusion protein. In a preferred embodiment, fusion proteins retain or have improved activity relative to the activity of the original protein, polypeptide or peptide prior to being fused to a heterologous protein, polypeptide, or peptide.

As used herein, the term “host cell” includes a particular subject cell transfected or transformed with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunomodulatory agent” and variations thereof including, but not limited to, immunomodulatory agents, immunomodulants or immunomodulatory drugs, refer to an agent that modulates a host's immune system. In a specific embodiment, an immunomodulatory agent is an agent that shifts one aspect of a subject's immune response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include an antibody that immunospecifically binds to Integrin αᵥβ₃. Immunomodulatory agents include, but are not limited to, small molecules, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules.

As used herein, the term “immunospecifically binds to an antigen” and analogous terms refer to peptides, polypeptides, proteins, fusion proteins and antibodies or fragments thereof that specifically bind to an antigen or a fragment and do; not specifically bind to other antigens. A peptide, polypeptide, protein, or antibody that immunospecifically binds to an antigen may bind to other peptides, polypeptides, or proteins with lower affinity as determined by, e.g., immunoassays, BIAcore, or other assays known in the art. Antibodies or fragments that immunospecifically bind to an antigen may cross-reactive with related antigens. Preferably, antibodies or fragments that immunospecifically bind to an antigen do not cross-react with other antigens.

As used herein, the term “immunospecifically binds to Integrin αᵥβ₃” and analogous terms refer to peptides, polypeptides, proteins, fusion proteins and antibodies or fragments thereof that specifically bind to Integrin αᵥβ₃ or a fragment thereof and do not specifically bind to other antigens. A peptide, polypeptide, protein, or antibody that immunospecifically binds to an Integrin αᵥβ₃ or a fragment thereof may bind to other peptides, polypeptides, or proteins with lower affinity as determined by, e.g., immunoassays, BIAcore, or other assays known in the art. Antibodies or fragments that immunospecifically bind to Integrin αᵥβ₃ or a fragment thereof may be cross-reactive with related antigens. Preferably, antibodies or fragments that immunospecifically bind to Integrin αᵥβ₃ or a fragment thereof do not cross-react with other antigens. Antibodies or fragments that immunospecifically bind to Integrin αᵥβ₃ or a fragment thereof can be identified, for example, by immunoassays, BIAcore, or other techniques known to those of skill in the art. An antibody or fragment thereof binds specifically to Integrin αᵥβ₃ or a fragment thereof when it binds to Integrin αᵥβ₃ or a fragment thereof with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

The term “α_{V}β₃” or “Integrin α_{V}β₃” refers to the heterodimer of the Integrin subunit α_{V} and the Integrin subunit β₃ and includes analogs, derivatives or fragments of the subunits of the heterodimer, and fusion proteins comprising the heterodimer Integrin α_{V}β₃, analogs, derivatives or a fragments of the subunits of the heterodimer. The Integrin α_{V}β₃ may be from any species. The nucleotide and/or amino acid sequences of Integrin α_{V}β₃ can be found in the literature or public databases, or the nucleotide and/or amino acid sequences can be determined using cloning and sequencing techniques known to one of skill in the art. For example, the nucleotide sequence of human Integrin α_{V}β₃ can be found in the GenBank database (see, e.g., Accession No. NM_{—}002210 for α_{V}, and Accession No. L28832 for 3). The amino acid sequence of human α_{V}β₃ can be found in the GenBank database (see, e.g., Accession No. AAA 61631 for α_{V}, and Accession No. S44360 for β₃). In a preferred embodiment, an Integrin α_{V}β₃ is human Integrin α_{V}β₃, an analog, derivative or a fragment thereof.

As used herein, the phrases “Integrin α_{V}β₃ antagonist” and “antagonist of Integrin α_{V}β₃” refers to any compound, including any protein, polypeptide, peptide, peptidomimetic, glycoprotein, antibody, antibody fragment, carbohydrate, nucleic acid, organic molecule, inorganic molecule, large molecule, or small molecule that blocks, inhibits, reduces or neutralizes the function, activity and/or expression of Integrin α_{V}β₃. Integrin α_{V}β₃ antagonists include in particular embodiments LM609 antibody and antibodies and antigen-binding fragments derived therefrom that likewise recognize Integrin α_{V}β₃, such as chimerized and humanized versions of LM609, for example the MEDI-522 (Vitaxin®; MedImmune, Inc.) antibody and antibodies that compete with LM609 or Vitaxin® for binding as well as other antibodies that bind to Integrin α_{V}β₃-Integrin α_{V}β₃ antagonists as used herein also refer to molecules encoded by the nucleotide or amino acid sequences corresponding to SEQ ID NO: 1, SEQ ID NO: 2, SEQ ID NO: 3 and SEQ ID NO: 4 and fragments thereof. Such anti-Integrin α_{V}β₃ antibodies have been described in International Publication Nos. WO 89/0515155, WO 98/33919 and WO 00/78815 and U.S. Pat. No. 5,753,230 which are incorporated in their entireties by reference. In additional embodiments, Integrin α_{V}β₃ antagonists also include antibodies immunospecific for specific epitopes identified by the screening methods of the present invention. In various embodiments, an Integrin α_{V}β₃ antagonist reduces the function, activity and/or expression of Integrin α_{V}β₃ by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the term “in combination” refers to the use of more than one therapies (e.g., more than one prophylactic agent and/or therapeutic agent). The use of the term “in combination” does not restrict the order in which therapies (e.g., prophylactic or therapeutic agents) are administered to a subject with cancer. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1-week, 2 weeks, 3 weeks, 4 weeks, 5 weeks, 6 weeks, 8 weeks, or 12 weeks after) the administration of a second therapy to a subject with cancer.

As used herein, the term “isolated” in the context of a compound refers to a compound that substantially free of chemical precursors or other chemicals when chemically synthesized. In a specific embodiment, the compound is 60%, 70%, 75%, 80%, 85%, 90%, 95%, or 99% free of other different compounds.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a preferred embodiment, an antibody of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule encoding an antibody of the invention is isolated.

As used herein, the phrase “low tolerance” refers to a state in which the patient suffers from side effects from a therapy so that the patient does not benefit from and/or will not continue therapy because of the adverse effects.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents) to “manage” a disease so as to prevent the progression or worsening of the disease.

As used herein, the terms “non-responsive” and refractory” describe patients treated with a currently available cancer therapy (e.g., chemotherapy, radiation therapy, surgery, hormonal therapy and/or biological therapy/immunotherapy), which is not clinically adequate to treat or relieve one or more symptoms associated with cancer. Typically, such patients suffer from severe, persistently active disease and require additional therapy to ameliorate the symptoms associated with their cancer. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. In various embodiments, a cancer is “non-responsive/refractory” when the number of cancer cells has not been significantly reduced, or has increased.

As used herein, the term “potentiate” refers to an improvement in the efficacy of a therapy (e.g., a therapeutic agent) at its common or approved dose.

As used herein, the terms “prophylactic agent” and “prophylactic agents” refer to any agent(s) which can be used in the prevention of the recurrence or spread of cancer. In certain embodiments, the term “prophylactic agent” refers to an Integrin α_{V}β₃ antagonist (e.g., an anti-Integrin α_{V}β₃ antibody such as Vitaxin®. In certain other embodiments, the term “prophylactic agent” does not refer an Integrin α_{V}β₃ antagonist. In yet other embodiments, the term “prophylactic agent” refers to an Integrin α_{V}β₃ antagonist and a cancer therapy other than an Integrin α_{V}β₃ antagonist. Preferably, a prophylactic agent is an agent which is known to be useful to, or has been or is currently being used to the prevent or impede the onset, development, progression and/or severity of cancer. Prophylactic agents may be characterized as different agents based upon one or more effects that the agents have in vitro and/or in vivo. For example, an anti-angiogenic agent may also be characterized as an immunomodulatory agent.

As used herein, the terms “prevent”, “preventing,” and “prevention” refer to the prevention of the recurrence, onset, or development of cancer or one or more symptoms thereof in a subject, said prevention resulting from a therapy (e.g., the administration of a prophylactic or therapeutic agent), or a combination therapy (e.g., the administration of a combination of prophylactic or therapeutic agents).

As used herein, the term “prophylactically effective amount” refers to the amount of a therapy (e.g., a prophylactic agent) which is sufficient to result in the prevention of the development, recurrence or onset of cancer or one or more symptoms thereof, or to enhance or improve the prophylactic effect(s) of another therapy (e.g., a prophylactic agent). A prophylactically effective amount may refer to the amount of a therapy (e.g., prophylactic agent) sufficient to prevent the recurrence or spread of cancer or the occurrence of cancer in a patient, including but not limited to those predisposed to cancer or previously exposed to carcinogens. A prophylactically effective amount may also refer to the amount of a therapy (e.g., a prophylactic agent) that provides a prophylactic benefit in the prevention of cancer. Further, a prophylactically effective amount with respect to a prophylactic agent of the invention means that amount of prophylactic agent alone, or in combination with other agents, that provides a prophylactic benefit in the prevention of cancer. Used in connection with an amount of an antagonist of Integrin α_{V}β₃, the term can encompass an amount that improves overall prophylaxis or enhances the prophylactic efficacy of or synergizes with another therapy (e.g., a prophylactic agent). Examples of suitable dosages of prophylactically effective amounts of agents are given infra in Section 5.8.2.

As used herein, a “prophylactic protocol” refers to a regimen for dosing and timing the administration of one or more therapies (e.g., one or more prophylactic agents) to achieve a prophylactic effect.

A used herein, a “protocol” includes dosing schedules and dosing regimens. The protocols herein are methods of use and include prophylactic and therapeutic protocols.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Other side effects include gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (57^{th} ed., 2003).

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey such as a cynomolgous monkey, a chimpanzee, and a human), and more preferably a human. In one embodiment, the subject is a farm animal (e.g., a horse, a cow, a pig, etc) or a pet (e.g., a dog or a cat). In another embodiment, the subject is refractory or non-responsive to current therapies for cancer or one or more symptoms thereof other than Integrin α_{V}β₃. In another embodiment, the subject is not an immunocompromised or immunosuppressed mammal, preferably a human (e.g., an HIV patient). In another embodiment, the subject is not a mammal, preferably a human, with a lymphocyte count under approximately 400 cells/mm³, preferably approximately 500 cells/mm³. In a preferred embodiment, the subject is a human.

As used herein, the term “synergistic” refers to a combination of therapies (e.g., prophylactic or therapeutic agents) which is more effective than the additive effects of any two or more single agents. For example, a synergistic effect of a combination of therapies (e.g., prophylactic or therapeutic agents) permits the use of lower dosages of one or more of the agents and/or less frequent administration of said therapies to a subject with cancer. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of cancer. In addition, a synergistic effect can result in improved efficacy of therapies in the prevention or treatment of cancer. Finally, synergistic effect of a combination of therapies may avoid or reduce adverse or unwanted side effects associated with the use of any single therapy.

As used herein, the terms “therapeutic agent” and “therapeutic agents” refer to any agent(s) which can be used in the treatment, management, or amelioration of cancer or one or more symptoms thereof. In certain embodiments, the term “therapeutic agent” refers to an Integrin α_{V}β₃ antagonist (e.g., an anti-Integrin α_{V}β₃ antibody such as Vitaxin®). In certain other embodiments, the term “therapeutic agent” does not refer an Integrin α_{V}β₃ antagonist. In yet other embodiments, the term “therapeutic agents” refers to an Integrin α_{V}β₃ antagonist and a cancer therapy other than an Integrin α_{V}β₃ antagonist. Preferably, a therapeutic agent is an agent which is known to be useful for, or has been or is currently being used for the treatment, management, or amelioration of cancer or one or more symptoms thereof. Therapeutic agents may be characterized as different agents based upon one or more effects the agents have in vivo and/or in vitro. For example, an anti-inflammatory agent may also be characterized as an immunomodulatory agent.

As used herein, the term “therapeutically effective amount” refers to that amount of a therapy (e.g., a therapeutic agent) which is sufficient to destroy, modify, control or remove primary, regional or metastatic cancer tissue, ameliorate cancer or one or more symptoms thereof, or prevent the advancement of cancer, cause regression of cancer, or enhance or improve the therapeutic effect(s) of another therapy (e.g., a therapeutic agent). A therapeutically effective amount may refer to the amount of a therapy (e.g., a therapeutic agent) sufficient to delay or minimize the spread of cancer. A therapeutically effective amount may also refer to the amount of a therapy (e.g., a therapeutic agent) that provides a therapeutic benefit in the treatment or management of cancer. Further, a therapeutically effective amount with respect to a therapeutic agent of the invention means that amount of therapeutic agent alone, or in combination with other therapies, that provides a therapeutic benefit in the treatment or management of cancer. Used in connection with an amount of an antagonist of Integrin α_{V}β₃, the term can encompass an amount that improves overall therapy, reduces or avoids unwanted effects, or enhances the therapeutic efficacy of or synergizes with another therapy (e.g., a therapeutic agent). In a specific embodiment, a therapeutically effective amount of a therapy (e.g., a therapeutic agent) may reduce the growth, formation, or increase in number of cells. In accordance with this embodiment, preferably, a therapeutically effective amount of a therapy (e.g., a therapeutic agent) reduces the growth, formation, or increase in number of cells by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS. Examples of suitable dosages of therapeutically effective amounts of therapeutic agents are given infra in Section 5.8.2.

As used herein, the term “therapeutic protocol” refers to a regimen for dosing and timing the administration of one or more therapies (e.g., one or more therapeutic agents) to achieve a therapeutic effect.

As used herein, the terms “therapies” and “therapy” can refer to any protocol(s), method(s) and/or agent(s) that can be used in the prevention, treatment, management or amelioration of cancer or one or more symptoms thereof. In certain embodiments, the terms “therapy” and “therapies” refer to cancer chemotherapy, radiation therapy, hormonal therapy, biological therapy, and/or other therapies useful for the prevention, management, or treatment of cancer known to an oncologist skilled in the art.

As used herein, the terms “treat”, “treatment” and “treating” refer to the eradication, removal, modification, or control of primary, regional, or metastatic cancer tissue, or the reduction or amelioration of the progression, severity, and/or duration of cancer or one or more symptoms thereof, or the amelioration of one or more symptoms thereof resulting from the administration of one or more therapies (e.g., prophylactic or therapeutic agents). In certain embodiments, such terms refer to a reduction of the growth, formation, and/or increase in number of cells. In other embodiments, such terms refer to the minimizing or delay of the spread of cancer resulting from the administration of one or more prophylactic or therapeutic agents to a subject with such a disease.

### 4. DESCRIPTION OF THE FIGURES

FIGS. 1A-1B: The nucleotide and deduced amino acid sequence of the variable region of the antibody Vitaxin®. FIG. 1A shows the nucleotide and deduced amino acid sequence for the Vitaxin® heavy chain variable region (SEQ ID NO: 1 and SEQ ID NO: 3, respectively) while FIG. 1B shows the nucleotide and deduced amino acid sequence for the Vitaxin® light chain variable region (SEQ ID NO: 2 and SEQ ID NO: 4, respectively).

FIG. 2. Flow cytometric analysis of antibody binding to tumor cell lines. The mouse mAb LM609 and the humanized and optimized mAb Vitaxin® were capable of binding to both human and hamster αᵥβ₃ while the humanized mAb demonstrated binding to human but not hamster αᵥβ₃. Rabbit αᵥβ₃ was recognized by Vitaxin® but not by Humanized anti-α_{V}β₃ and only poorly by LM609. Rat αᵥβ₃ was not recognized by any of the three antibodies but was detected by the anti-rat αᵥβ₃ control antibody.

FIG. 3. Flow cytometric analysis of binding of Vitaxin® to human β₃ transfected B16F10 cells. In order to determine the binding specificity of Vitaxin® for αᵥβ₃, the mouse melanoma line B16F10 was transfected with an expression vector encoding the human β₃ gene. Cells were analyzed by FACS for expression of β₃ expressed with the endogenous mouse αᵥ. Vitaxin® recognized the transfected cells, indicating that presentation of human β₃, presumably complexed to endogenous mouse αᵥ, is sufficient for antibody binding. This evidence suggests that the epitope recognized by Vitaxin® is contained within the β₃ protein.

FIG. 4. Binding of Integrin αᵥβ₃ specific antibodies to human β₃ containing rat residues. Transfection of human HEK293 cells with the human β₃ cDNA resulted in surface expression of αᵥβ₃ which was detectable by flow cytometry with LM609, humanized anti-Integrin α_{V}β₃ and Vitaxin®, but not with the anti-rat β₃ antibody. Mutations in the A and B regions of β₃ greatly reduced binding of the anti-human αᵥβ₃ antibodies but did not increase binding of the anti-rat β₃ antibody. Mutations in the C region did not affect binding of the anti-human antibodies but did increase binding of the anti-rat β₃ antibody. Interestingly, changing both the A and C region amino acids to rat residues eliminated binding of Vitaxin® and humanized anti-Integrin α_{V}β₃, but only marginally affected binding of LM609. Changes in the A and C region dramatically increased binding of the anti-rat β₃ antibody, however. Finally, by changing all three regions (A, B and C) to the corresponding rat residues, we were able to completely eliminate binding of LM609, humanized anti-Integrin α_{V}β₃ and Vitaxin®;

FIG. 5. Integrin αᵥβ₃ mutants and the binding affinity of antibodies to the amino acid-substituted Integrin αᵥβ₃ mutants

### 5. DETAILED DESCRIPTION OF THE INVENTION

The present invention encompasses treatment protocols that provide better prophylactic or therapeutic profiles than current single agent therapies or combination therapies for cancer. In particular, the invention encompasses the use of an antagonist of Integrin αᵥβ₃ for the prevention, management, treatment or amelioration of cancer or one or more symptoms thereof. The invention also encompasses treatment protocols that enhance the prophylactic or therapeutic effect of an antagonist of Integrin αᵥβ₃ (preferably, an antibody that immunospecifically binds to Integrin αᵥβ₃). Further, the invention encompasses the use of an antagonist of Integrin αᵥβ₃ (preferably, an antibody that immunospecifically binds to Integrin αᵥβ₃) conjugated or fused to a moiety (e.g., a therapeutic agent or drug) for preventing, managing, treating or ameliorating cancer or one or more symptoms thereof.

The invention provides methods for preventing, managing, treating or ameliorating cancer that has the potential to metastasize or has metastasized to an organ or tissue (e.g., bone) or one or more symptoms thereof, said methods comprising administering to a subject in need thereof one or more doses of a prophylactically or therapeutically amount of an antagonist of Integrin αᵥβ₃ (preferably, an antibody that immunospecifically binds to Integrin αᵥβ₃). In a specific embodiment, the invention provides methods for preventing, managing, treating or ameliorating cancer that has the potential to metastasize or has metastasized to the bone or one or more symptoms thereof, said methods comprising administering to a subject in need thereof one or more doses of a prophylactically or therapeutically effective amount of one or more antagonists of Integrin αᵥβ₃. In a preferred embodiment, the invention provides methods for preventing, managing, treating or ameliorating prostate cancer that has the potential to metastasize or has metastasized to the bone or one or more symptoms thereof, said methods comprising administering to a subject in need thereof one or more doses of a prophylactically or therapeutically effective amount of one or more antibodies or fragments thereof that immunospecifically bind to Integrin αᵥβ₃, preferably Vitaxin® or an antigen-binding fragment thereof.

The invention provides methods for preventing, managing, treating or ameliorating cancer or one or more symptoms thereof, said methods comprising administering to a subject in need thereof one or more doses of a prophylactically or therapeutically effective amount of an antagonist of Integrin αᵥβ₃ (preferably, an antibody that immunospecifically binds to Integrin αᵥβ₃ and more preferably Vitaxin® or an antigen-binding fragment thereof) fused or conjugated to a moiety (e.g., a therapeutic agent or drug). In a specific embodiment, the invention provides methods for preventing, managing, treating or ameliorating cancer that has the potential to metastasize or has metastasized to an organ or tissue (e.g., bone) or one or more symptoms thereof, said methods comprising administering to a subject in need thereof one or more doses of a prophylactically or therapeutically effective amount of an antagonist of Integrin αᵥβ₃ fused or conjugated to a moiety (e.g., a therapeutic agent or drug). In a more specific embodiment, the invention provides methods for preventing, managing, treating or ameliorating cancer that has the potential to metastasize or has metastasized to the bone or one or more symptoms thereof, said methods comprising administering to a subject in need thereof one or more doses of a prophylactically or therapeutically effective amount of one or more antibodies or fragments thereof that immunospecifically bind to Integrin αᵥβ₃ fused or conjugated to a moiety (e.g., a therapeutic agent or drug). In a preferred embodiment, the invention provides methods for prevention, managing, treating or ameliorating cancer that has the potential to metastasize or has metastasized to the bone or one or more symptoms thereof, said methods comprising administering to a subject in need thereof one or more doses of Vitaxin® or an antigen-binding fragment thereof fused or conjugated to a moiety. Examples of other moieties that an antagonist of Integrin αᵥβ₃ can be fused or conjugated to include, but are not limited to, those agents disclosed in Section 5.5.1 infra.

The present invention encompasses treatment protocols for cancer in which an antagonist of Integrin αᵥβ₃ is used in combination with a therapy other than an antagonist of Integrin αᵥβ₃. The invention is based, in part, on the recognition that antagonists of Integrin αᵥβ₃ potentiate and synergize with, enhance the effectiveness of, improve the tolerance of, and/or reduce the side effects caused by, other cancer therapies, including current standard and experimental chemotherapies. The combination therapies of the invention have additive potency, an additive therapeutic effect or a synergistic effect. The combination therapies of the invention enable lower dosages of the therapies (e.g., prophylactic or therapeutic agents) to be utilized in conjunction with antagonists of Integrin αᵥβ₃ for the prevention, management, treatment or amelioration of cancer and/or less frequent administration of such prophylactic or therapies to a subject with cancer to improve the quality of life of said subject and/or to achieve a prophylactic or therapeutic effect. The combination therapies of the invention enable lower dosages of one or more antagonists of Integrin αᵥβ₃ and/or less frequent administration of dosages of one or more antagonists of Integrin αᵥβ₃ to a subject with cancer to improve the quality of life of said subject and/or to achieve a prophylactic or therapeutic effect. Further, the combination therapies of the invention reduce or avoid unwanted or adverse side effects associated with the administration of current single agent therapies and/or existing combination therapies for cancer, which in turn improves patient compliance with the treatment protocol.

The present invention provides methods for preventing, managing, treating or ameliorating cancer or one or more symptoms thereof, said methods comprising administering to a subject in need thereof a dosage of a prophylactically or therapeutically effective amount of one or more antagonists of Integrin αᵥβ₃ in combination with the administration of a dosage of a prophylactically or therapeutically effective amount of one or more other agents useful for cancer therapy. The Integrin αᵥβ₃ antagonist utilized in accordance with such methods may or may not be conjugated or fused to a moiety (e.g., a therapeutic agent or drug). Examples of cancer therapies that can be used in combination with one or more antagonists of Integrin α_{V}β₃ include, but are not limited to those disclosed in Section 5.6 infra. In one embodiment, an antagonist of Integrin α_{V}β₃ is administered with another cancer therapy that works by the same mechanism as the antagonist of Integrin α_{V}β₃. In another embodiment, an antagonist of Integrin α_{V}β₃ is administered to a subject in need thereof in combination with another cancer therapy that works by a different mechanism than the antagonist of Integrin α_{V}β₃. By example and not by limitation, the cancer therapy can be apoptosis inducing, cytotoxic, antimitotic, tubulin stabilizing, microtubule formation inhibiting, topoisomerase active, anti-metabolic, or DNA interactive agents. In other embodiments, the cancer therapy administered to a subject in need thereof in combination with an antagonist of Integrin α_{V}β₃ is gene therapy based. In other embodiments, the therapy is another antibody that is not an antagonist of Integrin α_{V}β₃.

The invention provides methods for preventing, managing, treating or ameliorating cancer or one or more symptoms thereof, said methods comprising administering to a subject in need thereof one or more antagonists of Integrin α_{V}β₃ (preferably, antibodies that immunospecifically bind to Integrin α_{V}β₃) in combination with one or more therapies that are not cancer therapeutics (a.k.a., non-cancer therapies). The Integrin α_{V}β₃ antagonist utilized in accordance with such methods may or may not be conjugated or fused to a moiety (e.g., a therapeutic agent or drug). Examples of non-cancer therapies include, but are not limited to, anti-emetic agents, anti-fungal agents, anti-bacterial agents, anti-inflammatory agents, anti-viral agents and antibiotics.

In one embodiment, the antagonist of Integrin α_{V}β₃ used in accordance with the methods of the invention is an antibody or a fragment thereof that immunospecifically binds to Integrin α_{V}β₃. In a preferred embodiment, the antagonist of Integrin α_{V}β₃ used in accordance with the methods of the invention is an LM609 antibody or an antibody derived therefrom that immunospecifically α_{V}β₃, such as chimerized and humanized versions of LM609, for example the antibody Vitaxin®. Such antibodies have been described in International Publication Nos. WO 89/05155, WO 98/33919 and WO 00/78815 as well as U.S. Pat. No. 5,753,230, which are incorporated by reference herein in their entireties. In a particular embodiment, the antagonist of Integrin α_{V}β₃ used in accordance with the methods of the invention is an antibody or fragment thereof that competes with LM609 or Vitaxin® or an antigen-binding fragment thereof for binding to Integrin α_{V}β₃. In accordance with this embodiment, the antibody or fragment thereof that competes with LM609 or Vitaxin® or an antigen-binding fragment for binding to Integrin α_{V}β₃ preferably does not include the monoclonal antibody D12 or an antigen-binding fragment thereof disclosed in International Publication No. WO 98/40488.

In other embodiments, the invention provides antibodies that immunoreact with Ecr, the RGD-directed adhesion receptor found on the surface of both endothelial and melanoma cells. Encompassed by the invention are antibodies which are useful for inhibiting the ability of cells that contain the adhesion receptor to adhere to a subendothelial matrix composed of vitronectin, fibrinogen or von Willegrand factor. Also encompassed by the invention are antibodies that inhibit functional activity of Integrin α_{V}β₃ or inhibit Integrin α_{V}β₃-mediated pathologies. Accordingly, the invention provides antibodies useful for the inhibition of angiogenesis or the inhibition of other functions mediated or influenced by Integrin α_{V}β₃, including but not limited to cell proliferation, cell attachment, cell migration, granulation tissue development, and/or inflammation. Such antibodies have been described in International Publication Nos. WO 89/05155, WO 98/33919 and WO 00/78815 as well as U.S. Pat. No. 5,753,230, which are incorporated by reference herein in their entireties.

The invention provides protocols for the administration of an antagonist of Integrin α_{V}β₃ alone or in combination with other cancer or non-cancer therapies. The therapies (e.g., prophylactic or therapeutic agents) of the combination therapies of the present invention can be administered concomitantly or sequentially to a subject. The therapies (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., prophylactic or therapeutic agents), to avoid or reduce the side effects of one of the therapies (e.g., prophylactic or therapeutic agents), and/or to improve the efficacy of the therapy. The therapies (e.g., prophylactic or therapeutic agents) of the combination therapies of the invention can also be administered to a subject concurrently.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration. The pharmaceutical compositions of the invention may be used in accordance with the methods of the invention for the prevention, management, treatment or amelioration of cancer or one or more symptoms thereof. Preferably, the pharmaceutical compositions of the invention are sterile and in suitable form for a particular method of administration to a subject with cancer.

The methods and compositions of the invention are useful in preventing, managing, treating or ameliorating cancers, including, but not limited to, the cancers disclosed in Section 5.1.1.1 infra. Specific examples of cancers that can be prevented, managed, treated or ameliorated in accordance with the invention include, but are not limited to, cancer of the head, neck, eye, mouth, throat, esophagus, chest, bone, lung, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. In a specific embodiment, the methods and compositions of the invention are used for the prevention, management, treatment or amelioration of a primary or secondary cancer that expresses Integrin α_{V}β₃. In another embodiment, the methods and compositions of the invention are used for the prevention, management, treatment or amelioration of a primary or secondary cancer that does not express Integrin α_{V}β₃. In a preferred embodiment, the methods and compositions are used for the prevention, management, treatment or amelioration of a cancer that has the potential to metastasize or has metastasized to other tissues or organs (e.g., bone). In another preferred embodiment, the methods and compositions of the invention are used for the prevention, management, treatment or amelioration of lung cancer, prostate cancer, ovarian cancer, melanoma, bone cancer or breast cancer.

The methods and compositions of the invention are useful not only in untreated cancer patients but are also useful in the treatment of cancer patients partially or completely refractory to current standard and experimental cancer therapies, including, but not limited to, chemotherapies, hormonal therapies, biological therapies, radiation therapies, and/or surgery. In a specific embodiment, the methods and compositions of the invention are useful for the prevention, management, treatment or amelioration of cancer that has been shown to be or may be refractory or non-responsive to therapies other than those comprising the administration of Integrin α_{V}β₃ antagonists. In a preferred embodiment, the methods and compositions of the invention are useful for the prevention, management, treatment or amelioration of cancer that has been shown to be or may be refractory or non-responsive to therapies comprising the administration of an antibody or fragment thereof that immunospecifically binds to Integrin α_{V}β₃, preferably Vitaxin® or an antigen-binding fragment thereof. The methods and compositions of the invention are also useful for the prevention, management, treatment or amelioration of cancer or one or more symptoms thereof in patients that do not tolerate therapies other than antagonists for Integrin α_{V}β₃ (preferably antibodies or fragments thereof that immunospecifically bind to Integrin α_{V}β₃, preferably Vitaxin® or an antigen-binding fragment thereof) because of unwanted or adverse/side effects.

The invention also provides methods for screening for antagonists for Integrin α_{V}β₃. In certain embodiments, amino acid substitutions are made in the subunits of Integrin α_{V}β₃, for example to change the ligand specificity of the Integrin α_{V}β₃ and/or disrupt the heterodimerization of the subunit chains. In specific embodiments, such amino acid substitutions disrupt the specific interaction of certain antagonists of Integrin α_{V}β₃ with a particular Integrin α_{V}β₃ epitope. In a preferred embodiment, the amino acid substitutions are made within regions of an Integrin subunit that confer ligand binding specificity, preferably ligand binding specificity of LM609 and/or Vitaxin®. In a specific preferred embodiment, amino acids 171, 173 and 174 of the human β₃ subunit can be substituted, preferably with Gln, Ile and Lys, respectively, to disrupt binding to Vitaxin®. In another preferred embodiment, the amino acid substitutions are made in the β₃ subunit, preferably with Gln, Ile, Lys, Thr and Ser, at amino acids 171, 173, 174, 179, and 182, respectively. Accordingly, such amino acid substituted subunits of Integrin α_{V}β₃ can be used for screening antibodies with specific affinity for particular epitopes by identifying monoclonal antibodies that bind to wild type Integrin α_{V}β₃ but not the mutant form. In other embodiments, methods of the invention involve screening for antagonists that bind the region of amino acids 164-202 of human β₃ chain in the context of the heterodimer. In addition, the invention provides methods for identifying monoclonal antibodies that bind to the heterodimerized α_{V}β₃ but not the α_{V} or the β₃ chains when not included in a heterodimer. The antibodies identified utilizing such screening methods can be used for the prevention, treatment, management or amelioration of Integrin α_{V}β₃-mediated diseases and disorders or one or more symptoms thereof, including but not limited to cancer, inflammatory and autoimmune diseases either alone or in combination with other therapies. Preferably, these antibodies are not LM609, Vitaxin®, D12 or an antibody or antibody binding fragment thereof having the CDRs of LM609, Vitaxin® or D12 with no more than one, no more than two, no more than five, no more than eight, or no more than ten amino acid substitutions, deletions or insertions.

The invention provides methods of detecting, diagnosing and/or monitoring the progression of cancer utilizing one or more antagonists Integrin α_{V}β₃ (preferably, one or more antibodies that immunospecifically bind to Integrin α_{V}β₃ and more preferably Vitaxin® or an antigen-binding fragment thereof) conjugated or fused to a detectable agent. In particular, methods for facilitating the use of Integrin α_{V}β₃ antagonists in the analysis of Integrin α_{V}β₃ expression in biopsies of animal model and clinical study samples are also provided.

The present invention provides kits comprising one or more antagonists of Integrin α_{V}β₃ (preferably, one or more antibodies that immunospecifically bind to Integrin α_{V}β₃ and more preferably Vitaxin® or an antigen-binding fragment thereof) conjugated or fused to a detectable agent, therapeutic agent or drug, in one or more containers, for use in the prevention, treatment, management, amelioration, detection, monitoring or diagnosis of cancer. The invention also provides kits comprising one or more antagonists Integrin α_{V}β₃ (preferably, one or more antibodies that immunospecifically bind to Integrin αᵥβ₃ and more preferably Vitaxin® or an antigen-binding fragment thereof) in a first vial and one or more prophylactic or therapeutic agents, other than antagonists of Integrin α_{V}β₃, in a second vial for use in the prevention, treatment, management, amelioration, detection, monitoring or diagnosis of cancer. The invention also provides kits comprising one or more antagonists Integrin α_{V}β₃ (preferably, one or more antibodies that immunospecifically bind to Integrin α_{V}β₃ and more preferably Vitaxin® or an antigen-binding fragment thereof) conjugated or fused to a therapeutic agent or drug in a first vial and one or more prophylactic or therapeutic agents, other than antagonists of Integrin α_{V}β₃, in a second vial for use in the prevention, treatment, management, amelioration, detection, monitoring or diagnosis of cancer. The kits may further comprise packaging materials and/or instructions.

5.1 Prophylactic/Therapeutic Methods

The present invention provides methods for preventing, treating, managing or ameliorating cancer or one or more symptoms thereof, said methods comprising administering to a subject in need thereof one or more antagonists of Integrin α_{V}β₃ alone or in combination with one or more other therapies (e.g., one or more other prophylactic or therapeutic agents) useful in the prevention, treatment, management or amelioration of cancer or one or more symptoms thereof. In a specific embodiment, the Integrin α_{V}β₃ antagonists are conjugated to another moiety (e.g., a therapeutic agent or drug).

In one embodiment, an antagonist of Integrin α_{V}β₃ (preferably, one or more antibodies that immunospecifically bind to Integrin α_{V}β₃ and more preferably Vitaxin® or an antigen-binding fragment thereof) is administered to a subject using a dosing regimen that maintains the plasma concentration of the antagonist at a desirable level (e.g., about 0.1 to about 100 μg/ml), which continuously blocks the Integrin α_{V}β₃ activity. In a specific embodiment, the plasma concentration of the antagonist is maintained at 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors including, but not limited to, the nature of the cancer, the severity of the cancer, and the condition of the subject. In another embodiment, an antagonist of Integrin α_{V}β₃ (preferably, one or more antibodies that immunospecifically bind to Integrin α_{V}β₃ and more preferably Vitaxin® or an antigen-binding fragment thereof) is administered intermittently to a subject. In accordance with this embodiment, the antagonist may or may not be conjugated to a moiety (e.g., a therapeutic agent or a toxin).

In a specific embodiment, an antagonist of Integrin α_{V}β₃ (preferably, one or more antibodies that immunospecifically bind to Integrin α_{V}β₃ and more preferably Vitaxin® or an antigen-binding fragment thereof) is administered to a subject with bone cancer or a cancer that has metastasized to the bone using a dosing regimen that maintains the plasma concentration of the antagonist at a level that blocks at least 40%, preferably at least 50%, at least 55%, at least 60%, at least 65%, at least 70%, at least 75%, at least 80%, at least 85%, at least 90% or at least 95% of bone resorption. In a specific embodiment, the plasma concentration of the antagonist is maintained at about 0.1 μg/ml to about 100 μg/ml in a subject with bone cancer or a cancer that has metastasized to the bone.

In certain embodiments, an antagonist of Integrin α_{V}β₃ is administered to a subject, preferably a human, concurrently with one or more other therapies (e.g. prophylactic or therapeutic agents) useful for the treatment of cancer. The term “concurrently” is not limited to the administration of therapies at exactly the same time, but rather it is meant that an antagonist of Integrin α_{V}β₃ and the other therapy are administered to a subject in a sequence and within a time interval such that the Integrin α_{V}β₃ antagonist can act together with the other therapy(ies) to provide an increased benefit than if they were administered otherwise. For example, each therapy (e.g., Vitaxin®, chemotherapy, radiation therapy, hormonal therapy or biological therapy) may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In other embodiments, the Integrin α_{V}β₃ antagonist is administered to a subject before, concurrently or after surgery. Preferably the surgery completely removes localized tumors or reduces the size of large tumors. Surgery can also be done as a preventive measure or to relieve pain. In preferred embodiments, the Integrin α_{V}β₃ antagonist is Vitaxin® or an antigen-binding fragment thereof.

In various embodiments, the therapies (e.g., prophylactic or therapeutic agents) are administered to a subject less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two, three or more therapies (e.g., two, three or more prophylactic or therapeutic agents) are administered within the same patient visit.

In other embodiments, the therapies (e.g., prophylactic or therapeutic agents) are administered to a subject at about 2 to 4 days apart, at about 4 to 6 days apart, at about 1 week part, at about 1 to 2 weeks apart, or more than 2 weeks apart. In preferred embodiments, the therapies (e.g., prophylactic or therapeutic agents) are administered to a subject in a time frame where both therapies are still active. One skilled in the art would be able to determine such a time frame by determining, e.g., the half life of the administered therapy.

In certain embodiments, the therapies (e.g., prophylactic or therapeutic agents) of the invention are cyclically administered to a subject. Cycling therapy involves the administration of a first therapy (e.g., first agent) for a period of time, followed by the administration of a second therapy (e.g., a second agent) and/or third therapy (e.g., a second agent) for a period of time and repeating this sequential administration. Cycling therapy can reduce the development of resistance to one or more of the therapies, avoid or reduce the side effects of one of the therapies, and/or improves the efficacy of the therapy.

In certain embodiments, therapies (e.g., prophylactic or therapeutic agents) are administered to a subject in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In a preferred embodiment, Vitaxin® or an antigen-binding fragment thereof is administered to a subject once a week or every two weeks and chemotherapy is administered to said subject daily for several days. In another preferred embodiment, Vitaxin® or an antigen-binding fragment thereof is administered to a subject once a week or every two weeks and chemotherapy is administered to said subject continuously for several days to several weeks. In yet another preferred embodiments, Vitaxin® or an antigen-binding fragment thereof is administered to a subject once a week or every two weeks and chemotherapy is administered to said subject in sessions of a few hours to a few days. In accordance with these embodiments, there may be rest periods of a few weeks where no chemotherapy is administered to said subject.

In a preferred embodiment, Vitaxin® or an antigen-binding fragment thereof is administered to a subject once a week or every two weeks is administered once a week or every two weeks and radiation therapy is administered to said subject daily for several days. In another preferred embodiment, Vitaxin® or an antigen-binding fragment thereof is administered once a week or every two weeks to a subject and radiation therapy is administered to said subject three times per month for up to eight weeks. In another preferred embodiments, Vitaxin® or an antigen-binding fragment thereof is administered once a week or every two weeks is administered to a subject once a week or every two weeks and radiation therapy is administered to said subject one day per week for up to eight weeks. In accordance with these embodiments, there may be rest periods of a few weeks where no radiation is administered. In another preferred embodiment, Vitaxin® or an antigen-binding fragment thereof is administered to a subject once a week or every two weeks, hormonal therapy is administered to said subject daily, and biological therapy/immunotherapy is administered to said subject once a week or every two weeks.

In yet other embodiments, the therapeutic and prophylactic agents of the invention are administered to a subject in metronomic dosing regimens, either by continuous infusion or frequent administration without extended rest periods. Such metronomic administration can involve dosing at constant intervals without rest periods. Typically the prophylactic or therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the therapeutic and prophylactic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. In preferred embodiments, the Integrin α_{V}β₃ antagonist is Vitaxin® or an antigen-binding fragment thereof. The scheduling of such dose regimens can be optimized by the skilled oncologist.

In other embodiments, the therapies (e.g., prophylactic or therapeutic agents) are administered concurrently to a subject, i.e., individual doses of prophylactic or therapeutic agents are administered separately yet within a time interval such that the Integrin α_{V}β₃ antagonist can work together with the other agent or agents. For example, one prophylactic or therapeutic agent may be administered to a subject one time per week in combination with the another prophylactic or therapeutic agent that may be administered to said subject one time every two weeks or one time every three weeks. In other words, the dosing regimens for the prophylactic or therapeutic agents are carried out concurrently even if the prophylactic or therapeutic agents are not administered simultaneously or within the same patient visit. In preferred embodiments, the Integrin α_{V}β₃ antagonist is Vitaxin® or an antigen-binding fragment thereof.

When used in combination with other therapies (e.g., prophylactic and/or therapeutic agents), the Integrin α_{V}β₃ antagonist and the prophylactic and/or therapeutic agent can act additively or, more preferably, synergistically. In one embodiment, an Integrin αᵥβ₃ antagonist is administered to a subject concurrently with one or more prophylactic or therapeutic agents in the same pharmaceutical composition. In another embodiment, an Integrin α_{V}β₃ antagonist is administered to a subject concurrently with one or more other prophylactic or therapeutic agents in separate pharmaceutical compositions. In still another embodiment, an Integrin α_{V}β₃ antagonist is administered to a subject prior to or subsequent to administration of another prophylactic or therapeutic agent. The invention encompasses the administration of an antagonist of Integrin α_{V}β₃ to a subject in combination with other prophylactic or therapeutic agents by the same or different routes of administration, e.g., oral and parenteral. In certain embodiments, when an antagonist of Integrin α_{V}β₃ is administered to a subject concurrently with another prophylactic or therapeutic agent that potentially produces adverse side effects (including, but not limited to, toxicity), the prophylactic or therapeutic agent can advantageously be administered at a dose that falls below the threshold that the adverse side effect is elicited. In preferred embodiments, the Integrin α_{V}β₃ antagonist is Vitaxin® or an antigen-binding fragment thereof.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (57^{th} ed., 2003). Examples of suitable dosages of prophylactically or therapeutically effective amounts of agents are given infra in Section 5.8.2.

5.1.1 Patient Population

The invention provides methods for preventing, managing, treating, ameliorating cancer or one or more symptoms thereof comprising administrating to a subject a prophylactically or therapeutically effective amount of one or more Integrin α_{V}β₃ antagonists (preferably, one or more antibodies or fragments that immunospecifically bind to Integrin α_{V}β₃ and more preferably Vitaxin® or an antigen-binding fragment thereof), or a pharmaceutical composition comprising an Integrin α_{V}β₃ antagonist (preferably, one or more antibodies or fragments that immunospecifically bind to Integrin α_{V}β₃ and more preferably Vitaxin® or an antigen-binding fragment thereof). In a specific embodiment, the Integrin α_{V}β₃ antagonist(s) utilized in accordance with the methods of the invention is conjugated or fused to another moiety (e.g., a therapeutic agent or drug).

The invention also provides methods for preventing, managing, treating, ameliorating cancer or one or more symptoms thereof comprising administrating to a subject a prophylactically or therapeutically effective amount of one or more Integrin α_{V}β₃ antagonists (preferably, one or more antibodies or fragments that immunospecifically bind to Integrin α_{V}β₃ and more preferably Vitaxin® or an antigen-binding fragment thereof) and a cancer therapy. In particular, the invention provides methods for preventing, managing, treating, ameliorating cancer or one or more symptoms thereof comprising administrating to a subject a prophylactically or therapeutically effective amount of one or more Integrin α_{V}β₃ antagonists (preferably, one or more antibodies or fragments that immunospecifically bind to Integrin α_{V}β₃ and more preferably Vitaxin® or an antigen-binding fragment thereof) and a prophylactically or therapeutically effective amount of one or more therapies useful for the prevention, management, treatment or amelioration of cancer or one or more symptoms thereof (including, but not limited to the prophylactic or therapeutic agents listed in Section 5.6 hereinbelow). In a specific embodiment, the Integrin α_{V}β₃ antagonist(s) (preferably, one or more antibodies or fragments that immunospecifically bind to Integrin α_{V}β₃ and more preferably Vitaxin® or an antigen-binding fragment thereof) utilized in accordance with the methods of the invention is conjugated or fused to another moiety (e.g., a therapeutic agent or drug).

The invention encompasses methods for treating or managing patients undergoing or on any other treatment useful for the prevention, management, treatment or amelioration of cancer or one or more symptoms thereof. The invention encompasses methods for treating or managing a subject/patient suffering from or expected to suffer from cancer. Such patients may or may not have been previously treated for cancer. The invention also encompasses methods for treating or managing cancer in a subject undergoing cancer therapy before any adverse effects or intolerance occurs.

Integrin α_{V}β₃ antagonists or combination therapies described herein may be used as a first line, second line, third line or fourth line cancer treatment. The invention encompasses methods for treating or managing patients with cancer refractory to conventional therapies for such a cancer. A cancer may be determined to be refractory to a therapy means when at least some significant portion of the cancer cells are not killed or their cell division arrested in response to the therapy. Such a determination can be made either in vivo and/or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased. The invention encompasses methods for treating or managing patients with cancer refractory to existing single agent therapies for such a cancer.

The invention encompasses methods for treating or managing patients with cancer that are immunosuppressed as a result of having previously undergone other cancer therapies. The invention also encompasses methods for treating or managing patients who have proven refractory to other treatments but are no longer on these treatments. The invention also encompasses alternative methods for treating or managing patients in which chemotherapy, radiation therapy, hormonal therapy, and/or biological therapy/immunotherapy has proven or may prove too toxic, i.e., results in unacceptable or unbearable side effects, for the patient being treated or managed. The invention also encompasses methods for treating patients predisposed to cancer. The invention also encompasses methods for treating or managing patients with mean absolute lymphocyte cell counts of at least 500 cells/mm³, preferably at least 600 cells/mm³, more preferably at least 750 cells/mm³. The invention also encompasses methods for preventing the onset or development of one or more symptoms in patients with cancer. The invention also encompasses methods for ameliorating one or more symptoms in patients with incurable cancer, in particular hospice patients. Further, the invention provides methods for preventing cancer in patients who have been treated for cancer but have no disease activity.

In a preferred embodiment, the invention encompasses methods for managing or treating cancer patients that have undergone or are undergoing chemotherapy. In accordance with this embodiment, such patients include patients that have undergone or are undergoing radiation therapy, hormonal therapy, biological therapy/immunotherapy and/or surgery. Examples of chemotherapeutic agents that are used to treat cancer include, but not limited to methotrexate, taxol, mercaptopurine, thioguanine, hydroxyurea, cytarabine, cyclosporin A, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc.

In a specific embodiment, the invention encompasses methods for treating or managing cancer patients that have undergone or are undergoing radiation therapy. In accordance with this embodiment, such patients include patients that have undergone or are undergoing chemotherapy, hormonal therapy, biological therapy/immunotherapy and/or surgery. In another embodiment, the invention encompasses methods for treating or managing patients that have undergone or are undergoing hormonal therapy and/or biological therapy/immunotherapy. In accordance with this embodiment, such patients include patients that have undergone or are undergoing chemotherapy, radiation therapy and/or surgery.

Cancers that can be prevented, treated, managed or ameliorated in accordance with the methods of the invention include, but are not limited to, neoplasms, tumors, metastases, or any disease or disorder characterized by uncontrolled cell growth. The cancer may be a primary or metastatic cancer. The cancer may or may not express Integrin α_{V}β₃. In a specific embodiment, the cancer that is being managed, treated or ameliorated in accordance with the methods of the invention is a cancer expressing Integrin α_{V}β₃ that has metastasized to another organ or tissue. In a preferred embodiment, the cancer that is being managed, treated or ameliorated in accordance with the methods of the invention is a cancer expressing Integrin α_{V}β₃ that has metastasized to the bone. Specific examples of cancers that can be treated by the methods encompassed by the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the cancers disclosed in Section 5.1.1.1 infra.

5.1.1.1 Cancers

Examples of cancers that can be prevented, managed, treated or ameliorated in accordance with the methods invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). It is also contemplated that cancers caused by aberrations in apoptosis can also be treated by the methods and compositions of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

In certain embodiments, the cancer that is being prevented, managed, treated, or ameliorated in accordance with the methods of the invention expresses Integrin α_{V}β₃. In other embodiments, the cancer that is being prevented, managed, treated, or ameliorated in accordance with the methods of the invention overexpresses Integrin α_{V}β₃ relative to non-cancerous cells of the same type that the cancer originated from. In other embodiments, the cancer that is being prevented, managed, treated or ameliorated in accordance with the methods of the invention does not express Integrin α_{V}β₃. In a preferred embodiment, the cancer being managed, treated or ameliorated in accordance with the invention is associated with aberrant angiogenesis. As used herein, the term “aberrant angiogenesis” refers to any angiogenesis that is deviated from the normal process of angiogenesis, such as but not limited to, increased angiogenesis activity in a body, and angiogenesis at an abnormal location of the body.

In a preferred embodiment, the cancer being prevented, managed, treated or ameliorated in accordance with the methods of the invention is breast cancer, lung cancer, ovarian cancer, prostate cancer, colon cancer or melanoma. In another embodiment, the cancer that is being prevented, managed, treated or ameliorated in accordance with the methods of the invention are metastatic tumors including, but not limited to, tumors that have or may metastasize to the bone (non-limiting examples are prostate, breast and lung cancers that have metastasized or have the potential to metastasize to the bone), tumors that have or may metastasize to the lung, tumors that have or may metastasize to the brain, and tumors that have or may metastasize to other organs of a subject.

5.1.1.2 Treatment of Breast Cancer

In specific embodiments, patients with breast cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for breast cancer treatment or management including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), Herceptin®, tamoxifen, or the combination of tamoxifen and cytotoxic chemotherapy. In certain embodiments, patients with metastatic breast cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of taxanes such as docetaxel and paclitaxel. In other embodiments, a patients with node-positive, localized breast cancer are administered a prophylactically or therapeutically effective amount of one or more antagonists of Integrin α_{V}β₃ in combination with the administration of a prophylactically or therapeutically effective amount of taxanes plus standard doxorubicin and cyclophosphamide for adjuvant treatment of node-positive, localized breast cancer. In accordance with these embodiments, the Integrin α_{V}β₃ antagonist (preferably, one or more antibodies or fragments that immunospecifically bind to Integrin α_{V}β₃ and more preferably Vitaxin® or an antigen-binding fragment thereof) may or may not be conjugated or fused to a moiety such as a therapeutic agent or drug.

5.1.1.3 Treatment of Colon Cancer

In specific embodiments, patients with colon cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for colon cancer treatment or management including but not limited to: the combination of 5-FU and leucovorin, the combination of 5-FU and levamisole, irinotecan (CPT-11) or the combination of irinotecan, 5-FU and leucovorin (IFL). In accordance with these embodiments, the Integrin α_{V}β₃ antagonist (preferably, one or more antibodies or fragments that immunospecifically bind to Integrin α_{V}β₃ and more preferably Vitaxin® or an antigen-binding fragment thereof) may or may not be conjugated or fused to a moiety such as a therapeutic agent or drug.

5.1.1.4 Treatment of Prostate Cancer

In specific embodiments, patients with prostate cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for prostate cancer treatment or management including but not limited to: external-beam radiation therapy, interstitial implantation of radioisotopes (i.e., I¹²⁵, palladium, and Iridium), leuprolide or other LHRH agonists, non-steroidal antiandrogens (flutamide, nilutamide, and bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel. In accordance with these embodiments, the Integrin α_{V}β₃ antagonist (preferably, one or more antibodies or fragments that immunospecifically bind to Integrin α_{V}β₃ and more preferably Vitaxin® or an antigen-binding fragment thereof) may or may not be conjugated or fused to a moiety such as a therapeutic agent or drug.

5.1.1.5 Treatment of Melanoma

In specific embodiments, patients with melanoma are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for melanoma cancer treatment or management including but not limited to: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), interferon alpha (IFN-A), and interleukin-2 (IL-2). In an embodiment, a prophylactically or therapeutically effective amount of one or more antagonists of Integrin α_{V}β₃ preferably, one or more antibodies or fragments that immunospecifically bind to Integrin α_{V}β₃ and more preferably Vitaxin® or an antigen-binding fragment thereof) in combination with isolated hyperthermic limb perfusion (ILP) with melphalan (L-PAM), with or without tumor necrosis factor-alpha (TNT-alpha) can be administered to melanoma patients with multiple brain metastases, bone metastases, and spinal cord compression to achieve symptom relief and some shrinkage of the tumor with radiation therapy. In accordance with these embodiments, the Integrin α_{V}β₃ antagonist (preferably, one or more antibodies or fragments that immunospecifically bind to Integrin α_{V}β₃ and more preferably Vitaxin® or an antigen-binding fragment thereof) may or may not be conjugated or fused to a moiety such as a therapeutic agent or drug.

5.1.1.6 Treatment of Ovarian Cancer

In specific embodiments, patients with ovarian cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with a prophylactically or therapeutically effective amount of one or more other therapies useful for ovarian cancer treatment or management including, but not limited to: intraperitoneal radiation therapy, such as P³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-fluorouracil (5-FU) and leucovorin, etoposide, liposomal doxorubicin, gemcitabine or topotecan. In a particular embodiment, patients with ovarian cancer that is platinum-refractory are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of Taxol. The invention encompasses the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors.

5.1.1.7 Treatment of Lung Cancers

In specific embodiments, patients with small lung cell cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for lung cancer treatment or management including but not limited to: thoracic radiation therapy, cisplatin, vincristine, doxorubicin, and etoposide, alone or in combination, the combination of cyclophosphamide, doxorubicin, vincristine/etoposide, and cisplatin (CAV/EP), local palliation with endobronchial laser therapy, endobronchial stents, and/or brachytherapy.

In other specific embodiments, patients with non-small lung cell cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for lung cancer treatment or management including but not limited to: palliative radiation therapy, the combination of cisplatin, vinblastine and mitomycin, the combination of cisplatin and vinorelbine, paclitaxel, docetaxel or gemcitabine, the combination of carboplatin and paclitaxel, interstitial radiation therapy for endobronchial lesions or stereotactic radio surgery

5.1.1.8 Treatment of Bone Cancer and Bone Metastasis

In specific embodiments, patients with bone cancer and bone metastatic cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof). In other embodiments, patients with bone cancer and bone metastatic cancer are administered a prophylactically or therapeutically effective amount of one or more antagonists of Integrin α_{V}β₃ (preferably, one or more antibodies or fragments that immunospecifically bind to Integrin α_{V}β₃ and more preferably Vitaxin® or an antigen-binding fragment thereof) in combination with the administration of a prophylactically or therapeutically effective amount of one or more other therapies useful for bone cancer or metastatic bone cancer treatment or management, including but not limited to, peptides, polypeptides, proteins, fusion proteins, nucleic acid molecules, small molecules, mimetic agents, synthetic drugs, inorganic molecules, and organic molecules. In accordance with these embodiments, the Integrin α_{V}β₃ antagonist (preferably, one or more antibodies or fragments that immunospecifically bind to Integrin α_{V}β₃ and more preferably Vitaxin® or an antigen-binding fragment thereof) may or may not be conjugated or fused to a moiety such as a therapeutic agent or drug. Any agent or therapy which is know to be useful, or which has been used or is currently being used to treat bone cancer or metastatic bone cancer can be used in combination with an Integrin α_{V}β₃ antagonist (preferably, one or more antibodies or fragments that immunospecifically bind to Integrin α_{V}β₃ and more preferably Vitaxin® or an antigen-binding fragment thereof) or an Integrin α_{V}β₃ antagonist (preferably, one or more antibodies or fragments that immunospecifically bind to Integrin α_{V}β₃ and more preferably Vitaxin® or an antigen-binding fragment thereof) conjugated or fused to another moiety in accordance with the invention. Examples of such therapies include, but are not limited to, phosphate, aluminum hydroxide, aluminum carbonate gels, magnesium, vitamin D, calcitriol, vitamin D2 (ergocalciferol), vitamin D3 (cholecalciferol), calcium, lithium, glucocorticoids, biphosphonates or a pharmaceutically acceptable salt or ester thereof (non-limiting examples are alendronate, clodronate, etidronate, ibandronate, pamidronate, risedronate, tiludronate, and zoledronate), calcitonin, plicamycin (mithramycin), gallium nitrate, estrogens, progestins, estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of VEGF, antibodies to a growth factor or to a growth factor receptor, inhibitors of Flk-1/KDR, Flt-1, Tck/Tie-2, or Tie-1, cathepsin K inhibitors, inhibitors of osteoclast proton ATPase, inhibitors of urokinase plasminogen activator (u-PA), tumor-specific antibody-interleukin-2 fusion proteins, inhibitors of HMG-CoA reductase, prenylation inhibitors (non-limiting examples are farnesyl transferase inhibitor, geranylgeranyl transferase inhibitor or dual farnesyl/geranylgeranyl transferase inhibitors), parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, any agents used in cancer treatment (see section 5.6., infra), renal dialysis, surgery, or a combination thereof.

In a specific embodiment, patients with bone sarcomas are administered a prophylactically or therapeutically effective amount of one or Integrin α_{V}β₃ antagonists (preferably, one or more antibodies or fragments that immunospecifically bind to Integrin α_{V}β₃ and more preferably Vitaxin® or an antigen-binding fragment thereof in combination with a prophylactically or therapeutically effective amount of one or more other agents useful for bone sarcoma therapy including but not limited to: doxorubicin, ifosfamide, cisplatin, high-dose methotrexate, cyclophosphamide, etoposide, vincristine, dactinomycin, and surgery. In another specific embodiment, patients with tumor metastatic to bone are administered prophylactically or therapeutically of one or Integrin α_{V}β₃ antagonists in combination with a prophylactically or therapeutically effective amount of one or more other agents useful for bone metastatic tumor therapy including but not limited to: agents or therapies used in treatment of underlying malignancy (non-limiting examples are hormone inhibitors for prostate or breast cancer metastasized to bone and surgery), radiotherapy (non-limiting examples are strontium 89 and samarium 153, which are bone-seeking radionuclides that can exert antitumor effects and relieve symptoms), and bisphosphonates.

5.2 Antagonists of Integrin α_{V}β₃

The invention contemplates the administration of an effective amount of any Integrin α_{V}β₃ antagonists known in the art alone or in combination with the administration of an effective amount of one or more other agents useful for the treatment or prevention of cancer. By example and not by limitation, the invention encompasses administration of one or more Integrin α_{V}β₃ antagonists such as: the murine monoclonal LM609 (Scripps, International Publication Nos. WO 89/015155 and U.S. Pat. No. 5,753,230, which is incorporated herein by reference in its entirety); the humanized monoclonal antibody MEDI-522 (a.k.a. VITAXIN®, MedImmune, Inc., Gaithersburg, Md.; Wu et al., 1998, PNAS USA 95(11):6037-6042; International Publication No. WO 90/33919 and WO 00/78815, each of which is incorporated herein by reference in its entirety); D12 (International Publication No. WO 98/40488); anti-Integrin α_{V}β₃ antibody PDE 117-706 (ATCC access No. HB-12224), P112-4C1 (ATCC access No. HB-12225), P113-12A6 (ATCC access No. HB-12226), P112-11D2 (ATCC access No. HB-12227), P112-10D4 (ATCC access No. HB-12228) and P113-1F3 (ATCC access No. HB-12229) (G.D, Searle & Co.; International Publication No. WO 98/46264); 17661-37E and 17661-37E1-5 (USBiological); MON 2032 and 2033 (CalTag), ab7166 (BV3) and ab 7167 (BV4) (Abcam); and WOW-1 (Kiosses et al., Nature Cell Biology, 3:316-320); RGD-containing peptides such as Triflavin; small molecule peptidomimetic antagonists of Integrin α_{V}β₃ such as S836 (Searle) and S448 (Searle); Disintegrins and derivatives thereof, such as Accutin and genes or gene fragments such as del-1 gene (Progenitor) and PEX; a noncatalystic metalloproteinase fragment (Scripps) and Cilengitide (Merck KGA). The invention also contemplates the administration of an effective amount of the Integrin α_{V}β₃ antagonists as disclosed in the following U.S. patents and U.S. patent Application Publications: U.S. Pat. Nos. 6,472,403; 6,426,353; 6,416,964; 6,410,526; 6,358,970; 6,344,484; 6,316,412; 6,297,249; 6,294,549; 6,274,620; 6,268,378; 6,232,308; 6,211,184; 6,204,282; 6,193,968; 6,171,588; 6,160,099; 6,153,628; 6,130,231; 6,127,335; 6,100,423; 6,096,707; 6,090,944; 6,066,648; 6,048,861; 6,037,176; 6,017,926; 6,017,925; 5,985,278; 5,981,546; 5,981,478; 5,955,572; 5,952,341; 5,925,655; 5,919,792; 5,877,281; 5,852,210; 5,849,865; 5,849,692; 5,830,678; 5,843,906; 5,843,774; 5,817,457; 5,807,819; 5,792,745; 5,780,426; 5,773,646; 5,773,644; 5,773,412; 5,770,565; 5,767,071; 5,766,591; 5,760,029; 5,760,028; 5,759,996; 5,753,230; 5,710,159; 5,705,481; 5,693,612; 5,681,820; 5,652,110; 5,652,109; 5,578,704; 5,589,570; 5,523,209; 5,498,694; 5,478,725; 5,306,620; 5,262,520; 5,204,445; 5,196,511; 5,190,873; and 5,149,780; and U.S. Patent Application Publication Nos. 20020019402; 20020019387; 20020010176; 20020001840; 20010053853; 20010044535; 20010023242; 20010016645; 20010011125; and 20010001309, which are all herein incorporated by reference in their entireties.

In certain embodiments, the Integrin α_{V}β₃ antagonists do not include those disclosed in the following U.S. patents and U.S. patent Application Publications: U.S. Pat. Nos. 6,472,403; 6,426,353; 6,416,964; 6,410,526; 6,358,970; 6,344,484; 6,316,412; 6,297,249; 6,294,549; 6,274,620; 6,268,378; 6,232,308; 6,211,184; 6,204,282; 6,193,968; 6,171,588; 6,160,099; 6,153,628; 6,130,231; 6,127,335; 6,100,423; 6,096,707; 6,090,944; 6,066,648; 6,048,861; 6,037,176; 6,017,926; 6,017,925; 5,985,278; 5,981,546; 5,981,478; 5,955,572; 5,952,341; 5,925,655; 5,919,792; 5,877,281; 5,852,210; 5,849,865; 5,849,692; 5,830,678; 5,843,906; 5,843,774; 5,817,457; 5,807,819; 5,792,745; 5,780,426; 5,773,646; 5,773,644; 5,773,412; 5,770,565; 5,767,071; 5,766,591; 5,760,029; 5,760,028; 5,759,996; 5,753,230; 5,710,159; 5,705,481; 5,693,612; 5,681,820; 5,652,110; 5,652,109; 5,578,704; 5,589,570; 5,523,209; 5,498,694; 5,478,725; 5,306,620; 5,262,520; 5,204,445; 5,196,511; 5,190,873; and 5,149,780; and U.S. Patent Application Publication Nos. 20020019402; 20020019387; 20020010176; 20020001840; 20010053853; 20010044535; 20010023242; 20010016645; 20010011125; and 20010001309. In other embodiments, the Integrin α_{V}β₃ antagonists do not include 17661-37E and 17661-37E1-5 (USBiological); MON 2032 and 2033 (CalTag), D12 (International Publication No. WO 98/40488); anti-Integrin α_{V}β₃ antibody PDE 117-706 (ATCC access No. HB-12224), P112-4C1 (ATCC access No. HB-12225), P113-12A6 (ATCC access No. HB-12226), P112-11D2 (ATCC access No. HB-12227), P112-10D4 (ATCC access No. HB-12228) and P113-1F3 (ATCC access No. HB-12229) (G.D, Searle & Co.; International Publication No. WO 98/46264); ab7166 (BV3) and ab 7167 (BV4) (Abcam); and WOW-1 (Kiosses et al., Nature Cell Biology, 3:316-320); RGD-containing peptides such as Triflavin; small molecule peptidomimetic antagonists of Integrin α_{V}β₃ such as S836 (Searle) and S448 (Searle); Disintegrins and derivatives thereof, such as Accutin and genes or gene fragments such as del-1 gene (Progenitor) and PEX; a noncatalystic metalloproteinase fragment (Scripps) and Cilengitide (Merck KGA).

In a preferred embodiment, antagonists of Integrin α_{V}β₃ are antibodies. In a more preferred embodiment, antagonists of Integrin α_{V}β₃ are Vitaxin®, its derivatives, analogs, and epitope-binding fragments thereof (such as but not limited to, those disclosed in International Publication Nos. WO 89/05155, WO 98/33919, and WO 0078815).

In a particular embodiment, antagonists of Integrin α_{V}β₃ are antibodies or fragments thereof that compete with Vitaxin® or an antigen-binding fragment thereof for binding to Integrin α_{V}β₃.

5.3 Methods of Screening for Integrin α_{V}β₃ Antagonists

The invention provides methods for identifying antagonist of Integrin α_{V}β₃, particularly for antibodies that specifically bind to the same epitope as Vitaxin® and/or LM609. In part, the present inventors have found that mutation of residues 171, 173 and/or 174 of the human β₃ chain disrupt binding of Vitaxin® and/or LM609 antibodies to the Integrin α_{V}β₃ heterodimer. The present inventors have also found that although Vitaxin® and LM609 do not bind to mouse Integrin α_{V}β₃, Vitaxin® and LM609 do bind to a modified mouse Integrin α_{V}β₃ in which the region of the mouse β chain that corresponds to amino acids 164-202 of the human β chain are replaced with amino acids 164-202 of the human β chain. In certain embodiments, amino acid substitutions are made in the subunits of Integrin α_{V}β₃, for example to change the ligand specificity of the Integrin α_{V}β₃ and/or disrupt the heterodimerization of the subunit chains. Preferably the Integrin α_{V}β₃ is human. In specific embodiments, such amino acid substitutions disrupt the specific interaction of certain antagonists of Integrin α_{V}β₃ with a particular Integrin α_{V}β₃ epitope. In a preferred embodiment, the amino acid substitutions are made within regions of an Integrin subunit that confers ligand binding specificity, preferably ligand binding specificity of LM609 and/or Vitaxin®, particularly residues 164-202 of human β₃. Alternatively, mouse β chain residues corresponding to residues 164-202 of the human β₃ chain are replaced with the residues 164-202 of the human β₃ chain. Such mouse-human chimeras can be used to screen for antagonists that bind to the region 164-202 of human β₃ but not to mouse Integrin α_{V}β₃.

In preferred embodiments, the amino acid substitutions are made in the β₃ subunit. In certain embodiments, human β₃ residues are substituted with rat residues as described in Table 1. In one embodiment, the substitution of human residue Glu to rat residue Gln at position 171 (“Mutation A”) disrupts Integrin α_{V}β₃ binding to LM609. This same change disrupts binding to Vitaxin®. In another embodiment, the substitution of human residue Leu and Glu to rat residues Ile, and Lys at positions 173 and 174, respectively (“Mutation B”) both disrupt binding to Vitaxin® and increase binding to an anti rat β₃ antibody. In yet another embodiment, the substitution of human residues Asp and Thr to rat residues Thr and Ser at positions 179 and 182 respectively (“Mutation C”) confer binding specificity to an anti-rat β₃ antibody. Mutations A and C combined (three substituted residues) confer binding specificity for the mouse-anti-rat β₃ antibody and disrupts binding to Vitaxin®. In a specific preferred embodiment, amino acids 171, 173 and 174 can be substituted to disrupt binding to Vitaxin®. In an alternate preferred embodiment, amino acids 171, 173, 174, 179 and 182 can be substituted to disrupt binding of Integrin α_{V}β₃ to LM609 and humanized anti-Integrin αᵥβ₃ antibodies such as Vitaxin®. Such substitutions preferred examples but not limiting. Such substituted subunits are merely exemplary and not limiting. Any Integrin α_{V}β₃ regions identified to be responsible for antibody binding can be altered with substituted, deleted or inserted residues to characterize binding specificity of various antibodies and to screen for antibodies with the same a similar binding specificity.

Amino acid substituted subunits of Integrin α_{V}β₃ can be used for screening antibodies with specific affinity for particular epitopes by identifying monoclonal antibodies that bind to wild type Integrin α_{V}β₃ but not the altered form, or that bind mouse α_{V}β₃ integrins with a region substituted with the corresponding region from the human α_{V}β₃ but do not bind to wild type mouse Integrin α_{V}β₃. In addition, the invention provides methods for identifying monoclonal antibodies that bind to the heterodimerized α_{V}β₃ but not the α_{V} or the β₃ chains when not included in a heterodimer. Such screening can be accomplished by any routine method for assaying antibody specificity known in the art, for example, using cell lines that do not express wild type Integrin α_{V}β₃ to recombinantly express the mutant Integrin α_{V}β₃ or individual α_{V} or β₃ chains. The antibodies identified from such screening methods can be useful for the prevention, management and treatment of Integrin α_{V}β₃-mediated diseases and disorders, including but not limited to inflammatory diseases, autoimmune diseases, bone metabolism related disorders, angiogenic related disorders, disorders related to aberrant expression and/or activity of α_{V}β₃, and cancer. Such antibodies can be used in the methods and compositions of the present invention. Preferably, these antibodies are not LM609, Vitaxin®, D12 or an antibody or antibody binding fragment thereof having the CDRs (or one, two, three, four or five of the CDRs or CDR3 of the heavy chain) of LM609, Vitaxin® or D12 with no more than one, no more than two, no more than five, no more than eight, or no more than ten amino acid substitutions, deletions or insertions.

TABLE 1

| Human Beta3 | Mutation A | Mutation B | | Mutation C | |
|---|---|---|---|---|---|
| mutants | (Glu-Gln) | (Leu-Ile), (Glu-Lys) | | (Asp-Thr), (Thr-Ser) | |
| A1(A, C) | E171Q | | | D179T | T182S |
| A6 | E171Q | | | | |
| B1 | | L173 I | E174K | | |
| C14 | | | | D179T | T182S |
| C16 | | | | D179T | T182S |
| ABC17 | E171Q | L173 I | E174K | D179T | T182S |

5.4 Antibodies that Immunospecifically Bind to Integrin α_{V}β₃

As discussed above, the invention encompasses administration of antibodies or fragments thereof that immunospecifically bind to Integrin α_{V}β₃. The invention encompasses the administration of Integrin α_{V}β₃ monoclonal and polyclonal antibodies including, but not limited to, LM609 (Scripps), the murine monoclonal LM609 (International Publication Nos. WO 89/015155 and U.S. Pat. No. 5,753,230, which is incorporated herein by reference in its entirety); the humanized monoclonal antibody MEDI-522 (a.k.a. VITAXIN®, MedImmune, Inc., Gaithersburg, Md.; Wu et al., 1998, PNAS USA 95(11):6037-6042; International Publication No. WO 90/33919 and WO 00/78815, each of which is incorporated herein by reference in its entirety); D12 (International Publication No. WO 98/40488); anti-Integrin α_{V}β₃ antibody PDE 117-706 (ATCC access No. HB-12224), P112-4C1 (ATCC access No. HB-12225), P113-12A6 (ATCC access No. HB-12226), P112-11D2 (ATCC access No. HB-12227), P112-10D4 (ATCC access No. HB-12228) and P113-1F3 (ATCC access No. HB-12229). (G.D, Searle & Co., International Publication No. WO 98/46264); 17661-37E and 17661-37E1-5 (USBiological), MON 2032 and 2033 (CalTag), ab7166 (BV3) and ab 7167 (BV4) (Abcam), WOW-1 (Kiosses et al., Nature Cell Biology, 3:316-320), and analogs, derivatives, or fragments thereof. In a preferred embodiment, the antibody is Vitaxin®, which is a humanized blocking monoclonal antibody that binds Integrin α_{V}β₃ or an antigen-binding fragment thereof. Set forth below, is a more detailed description of the antibodies encompassed within the various aspects of the invention.

Antibodies used in the methods of the invention include, but are not limited to, monoclonal antibodies, synthetic antibodies, multispecific antibodies, human antibodies, camelized antibodies, humanized antibodies, chimeric antibodies, single-chain Fvs (scFv), single domain antibodies, single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to Integrin α_{V}β₃. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of a Integrin α_{V}β₃ polypeptide or may immuno specifically bind to both an Integrin α_{V}β₃ polypeptide as well a heterologous epitope, such as a heterologous polypeptide or solid support material. See, e.g., International Publication Nos. WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., J. Immunol. 148:1547-1553 (1992).

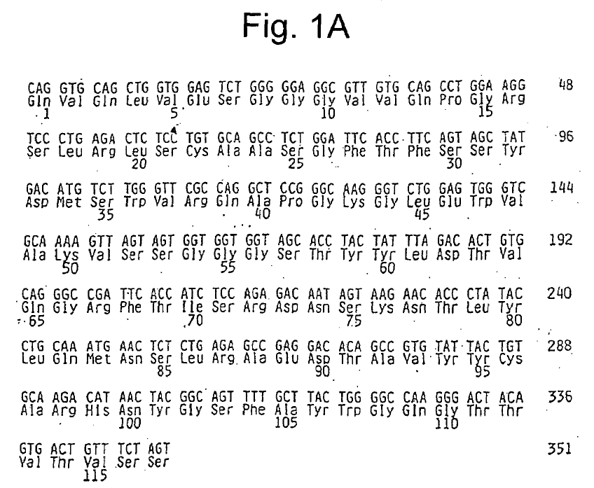

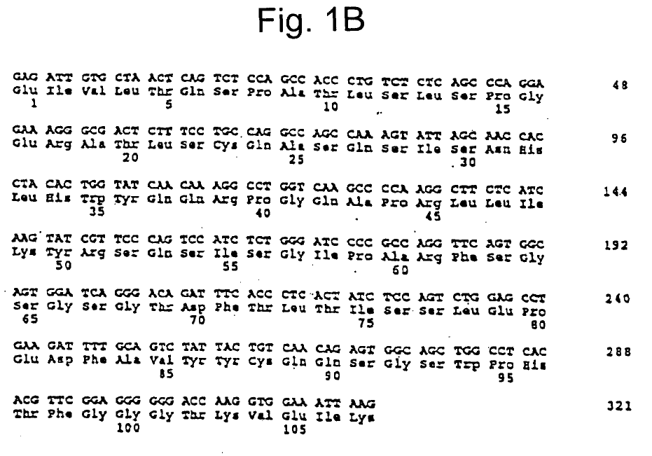

In a specific embodiment, an antibody used in the methods of the present invention is Vitaxin® or an antigen-binding fragment thereof (e.g., one or more complementarity determining regions (CDRs) of Vitaxin®. The amino acid sequence of Vitaxin® is disclosed, e.g., in International Publication Nos. WO 98/33919, WO 00/78815, and WO 02/070007, U.S. application Ser. No. 09/339,922, each of which is incorporated herein by reference in its entirety. The amino acid sequences for the heavy chain variable region and light chain variable region are provided herein as SEQ ID NO:3 and SEQ ID NO:4, respectively (FIGS. 1A and 1B). The nucleotide sequence for the heavy chain variable and light chain variable region are provided herein as SEQ ID NO: 1 and SEQ ID NO:2, respectively (FIGS. 1A and 1B). In another embodiment, an antibody used in the methods of the present invention binds to the same epitope as Vitaxin® or competes with Vitaxin® for binding to Integrin α_{V}β₃. In an alternative embodiment, an antibody that immunospecifically binds to Integrin α_{V}β₃ is not Vitaxin® or an antigen-binding fragment of Vitaxin®.

The present invention encompasses the use of antibodies that immunospecifically bind to Integrin αᵥβ₃, said antibodies comprising a variable heavy (“VH”) domain having an amino acid sequence of the VH domain for LM609 or VITAXIN®. The present invention also encompasses the use of antibodies that immunospecifically bind to Integrin αᵥβ₃, said antibodies comprising a variable light (“VL”) domain having an amino acid sequence of the VL domain for LM609 or VITAXIN®. The invention further encompasses the use of antibodies that immunospecifically bind to Integrin αᵥβ₃, said antibodies comprising a VH domain disclosed herein combined with a VL domain disclosed herein, or other VL domain. The present invention further encompasses antibodies that immunospecifically bind to Integrin αᵥβ₃, said antibodies comprising a VL domain disclosed herein combined with a VH domain disclosed herein, or other VH domain.

The present invention encompasses the use of antibodies that immunospecifically bind to Integrin αᵥβ₃, said antibodies comprising a VH CDR having an amino acid sequence of any one of the VH CDRs listed in Table 2 infra. The present invention also encompasses the use of antibodies that immunospecifically bind to Integrin αᵥβ₃, said antibodies comprising a VL CDR having an amino acid sequence of any one of the VL CDRs listed in Table 2 infra. The present invention also encompasses the use of antibodies that immunospecifically bind to Integrin αᵥβ₃, said antibodies comprising one or more VH CDRs and one or more VL CDRs listed in Table 2. In particular, the invention encompasses the use of antibodies that immunospecifically binds to Integrin αᵥβ₃, said antibodies comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 infra.

TABLE 2
CDR Sequences Of LM609

| CDR | Sequence | SEQ ID NO: |
|---|---|---|
| VH1 | SYDMS | 5 |
| VH2 | KVSSGGG | 6 |
| VH3 | HNYGSFAY | 7 |
| VL1 | QASQSISNHLH | 8 |
| VL2 | YRSQSIS | 9 |
| VL3 | QQSGSWPHT | 10 |

The antibodies used in the methods of the invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also encompasses antibodies or fragments thereof that compete with Vitaxin® or LM609 or an antigen-binding fragment thereof for binding to Integrin α_{V}β₃. Competition assays which can be used to identify such antibodies are well-known to one of skilled in the art. In a particular embodiment, 1 μg/ml of an antibody prevents 75%, 80%, 85% or 90% of ORIGEN TAG labeled LM609 or Vitaxin® from binding to biotin-labeled Integrin α_{V}β₃ as measured by well-known ORIGEN analysis. In another embodiment, the invention encompasses antibodies or fragments other than those disclosed in WO 98/40488 that compete with Vitaxin®. LM609 or an antigen-binding fragment thereof for binding to Integrin α_{V}β₃.

The present invention also provides antibodies of the invention or fragments thereof that comprise a framework region known to those of skill in the art. Preferably, the fragment region of an antibody of the invention or fragment thereof is human or humanized. In a specific embodiment, an antibody of the invention or fragment thereof comprises the framework region of Vitaxin® and/or one or more CDRs from Vitaxin®.

The present invention encompasses the use of antibodies or antibody fragments comprising the amino acid sequence of Vitaxin® with mutations (e.g., one or more amino acid substitutions) in the framework or variable regions. Preferably, mutations in these antibodies maintain or enhance the avidity and/or affinity of the antibodies for the Integrin α_{V}β₃ to which they immunospecifically bind. Standard techniques known to those skilled in the art (e.g., immunoassays) can be used to assay the affinity of an antibody for a particular antigen.

The present invention encompasses the use of a nucleic acid molecule(s), generally isolated, encoding an antibody that immunospecifically binds to Integrin α_{V}β₃. In a specific embodiment, an isolated nucleic acid molecule encodes an antibody that immunospecifically binds to Integrin α_{V}β₃, said antibody having the amino acid sequence of LM609 or Vitaxin®. In another embodiment, an isolated nucleic acid molecule encodes an antibody that immunospecifically binds to Integrin αᵥβ₃, said antibody comprising a VH domain having the amino acid sequence of the VH domain of LM609 or Vitaxin®M. In another embodiment, an isolated nucleic acid molecule encodes an antibody that immunospecifically binds to Integrin α_{V}β₃, said antibody comprising a VL domain having the amino acid sequence of the VL domain of LM609 or Vitaxin®.

The invention encompasses the use of an isolated nucleic acid molecule encoding an antibody that immunospecifically binds to Integrin α_{V}β₃, said antibody comprising a VH CDR having the amino acid sequence of any of the VH CDRs listed in Table 2, supra. In particular, the invention encompasses the use of an isolated nucleic acid molecule encoding an antibody that immunospecifically binds to Integrin α_{V}β₃, said antibody comprising one, two, or more VH CDRs having the amino acid sequence of any of the VH CDRs listed in Table 2, supra.

The present invention encompasses the use of an isolated nucleic acid molecule encoding an antibody that immunospecifically binds to Integrin αᵥβ₃, said antibody comprising a VL CDR having an amino acid sequence of any of the VL CDRs listed in Table 2, supra. In particular, the invention encompasses the use of an isolated nucleic acid molecule encoding an antibody that immunospecifically binds to Integrin αᵥβ₃, said antibody comprising one, two or more VL CDRs having the amino acid sequence of any of the VL CDRs listed in Table 2, supra.

The present invention encompasses the use of antibodies that immunospecifically bind to Integrin αᵥβ₃, said antibodies comprising derivatives of the VH domains, VH CDRs, VL domains, or VL CDRs described herein that immunospecifically bind to Integrin αᵥβ₃. Standard techniques known to those of skill in the art can be used to introduce mutations (e.g., additions, deletions, and/or substitutions) in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues (i.e., amino acid residues which are not critical for the antibody to immunospecifically bind to Integrin αᵥβ₃). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention encompasses the use of antibodies that immunospecifically bind to Integrin αᵥβ₃, said antibodies comprising the amino acid sequence of LM609 or Vitaxin® with one or more amino acid residue substitutions in the variable light (VL) domain and/or variable heavy (VH) domain. The present invention also encompasses the use of antibodies that immunospecifically bind to Integrin αᵥβ₃, said antibodies comprising the amino acid sequence of LM609 or Vitaxin®g) with one or more amino acid residue substitutions in one or more VL CDRs and/or one or more VH1 CDRs. The antibody generated by introducing substitutions in the VH domain, VH CDRs, VL domain and/or VL CDRs of LM609 or Vitaxin® can be tested in vitro and in vivo, for example, for its ability to bind to Integrin αᵥβ₃ (by, e.g., immunoassays including, but not limited to ELISAs and BIAcore), or for its ability to prevent, treat, manage or ameliorate cancer or one or more symptoms thereof.

The present invention also encompasses the use of antibodies or fragments thereof that immunospecifically bind to Integrin α_{V}β₃ or a fragment thereof, said antibodies or antibody fragments comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of Vitaxin® (i.e., SEQ ID NO:3 and/or SEQ ID NO:4). The present invention further encompasses the use of antibodies or fragments thereof that immunospecifically bind to Integrin α_{V}β₃ or a fragment thereof, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of Vitaxin®. The determination of percent identity of two amino acid sequences can be determined by any method known to one skilled in the art, including BLAST protein searches.

The present invention also encompasses the use of antibodies or antibody fragments that immunospecifically bind to Integrin α_{V}β₃ or fragments thereof, where said antibodies or antibody fragments are encoded by a nucleotide sequence that hybridizes to the nucleotide sequence of Vitaxin® under stringent conditions. In a preferred embodiment, the invention encompasses the use of an antibody or fragment thereof that immunospecifically binds to Integrin α_{V}β₃ or a fragment thereof, said antibody or antibody fragment comprising a variable light and/or variable heavy chain encoded by a nucleotide sequence that hybridizes under stringent conditions to the nucleotide sequence of the variable light and/or variable heavy chain of Vitaxin® (i.e., SEQ ID NO:1 and/or SEQ ID NO:2). In another preferred embodiment, the invention encompasses the use of an antibody or fragment thereof that immunospecifically binds to Integrin α_{V}β₃ or a fragment thereof, said antibody or antibody fragment comprising one or more CDRs encoded by a nucleotide sequence that hybridizes under stringent conditions to the nucleotide sequence of one or more CDRs of Vitaxin®. Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The methods of the present invention also encompass the use of antibodies or fragments thereof that have half-lives in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., PCT Publication No. WO 97/34631 and co-pending Provisional Application No. 60/254,884 filed Dec. 12, 2000 entitled “Molecules With Extended Half-Lives, Compositions and Uses Thereof,” which are incorporated herein by reference in their entireties). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

Further, antibodies can be conjugated to albumin in order to make the antibody or antibody fragment more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

5.4.1 Antibody Conjugates

The present invention encompasses the use of antibodies or fragments thereof conjugated or fused to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses the use of antibodies or fragments thereof recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes compositions comprising heterologous proteins, peptides or polypeptides fused or conjugated to antibody fragments. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof. Methods for fusing or conjugating polypeptides to antibody portions are well-known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins, e.g., of Vitaxin® or other anti integrin α_{V}β₃ antibodies, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to Integrin α_{V}β₃ may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments, analogs or derivatives thereof conjugated to a diagnostic or detectable agent. Such antibodies can be useful for monitoring or prognosing the development or progression of a cancer as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In, ¹¹²In, ¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹ Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴² Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, procaine, tetracaine, lidocaine, propranolol, and puromycin and analogs or homologs thereof) and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-10; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin) and pharmaceutically acceptable salts, solvates, clathrates, and prodrugs thereof. See, e.g., Rothenberg, M. L., Annals of Oncology 8:837-855 (1997); and Moreau, P., et al., J. Med. Chem. 41:1631-1640 (1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies, and adenosine deaminase inhibitors (e.g., Fludarabine phosphate and 2-Chlorodeoxyadenosine). Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, solastatin 10, see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), anti-mitotic agents (e.g., vincristine and vinblastine), hormones (e.g., glucocorticoids, progestatins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin; bulgarein; DNA minor groove binders such as Hoescht dye 33342 and Hoechst dye 33258; nitidine; fagaronine; epiberberine; coralyne; beta-lapachone; BC-4-1; and pharmaceutically acceptable salts, solvates, clathrates, and prodrugs thereof. See, e.g., Rothenberg, M. L., Annals of Oncology 8:837-855 (1997); and Moreau, P., et al., J. Med. Chem. 41:1631-1640 (1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies (e.g., rituximab (Rituxan®), calicheamycin (Mylotarg®), ibritumomab tiuxetan (Zevalin®), and tositumomab (Bexxar®), and adnosine deaminase inhibitors (e.g., Fludarabine phosphate and 2-Chlorodeoxyadenosine).

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International publication No. WO 97/33899), AIM II (see, International Publication No. WO 97/34911), Fas Ligand (Takahashi et al., 1994, J. Immunol., 6:1567-1574), and VEGI (see, International publication No. WO 99/23105), a thrombotic agent or an anti-angiogenic agent, e.g., angiostatin, endostatin or a component of the coagulation pathway (e.g., tissue factor); or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid. fibrinopeptides A and B from the α and β chains of fibrinogen, fibrin monomer).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

The therapeutic moiety or drug conjugated to an antibody or fragment thereof that immunospecifically binds to Integrin α_{V}β₃ should be chosen to achieve the desired prophylactic or therapeutic effect(s) for a particular disorder in a subject. A clinician or other medical personnel should consider the following when deciding on which therapeutic moiety or drug to conjugate to an antibody or fragment thereof that immunospecifically binds to Integrin α_{V}β₃: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.4.2 Methods Of Producing Antibodies

The antibodies or fragments thereof can be produced by any method known in the art for the synthesis of antibodies, in particular, by chemical synthesis or preferably, by recombinant expression techniques.

Polyclonal antibodies to Integrin α_{V}β₃ can be produced by various procedures well known in the art. For example, Integrin α_{V}β₃ or immunogenic fragments thereof can be administered to various host animals including, but not limited to, rabbits, mice, rats, etc. to induce the production of sera containing polyclonal antibodies specific for Integrin αᵥβ₃. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with Integrin α_{V}β₃ and once an immune response is detected, e.g., antibodies specific for Integrin α_{V}β₃ are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with Integrin α_{V}β₃ with myeloma cells and then screening the hybridomas resulting from the fusion for hybridoma clones that secrete an antibody able to bind Integrin α_{V}β₃.

Antibody fragments which recognize specific Integrin α_{V}β₃ epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the Integrin α_{V}β₃ epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express fall-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′).sub.2, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant-domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morea et al., Methods 20(3):267-79 (2000), Baca et al., J. Biol. Chem. 272(16):10678-84 (1997), Roguska et al, Protein Eng. 9(10):895-904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies of the invention can, in turn, be utilized to generate anti idiotype antibodies that “mimic” Integrin α_{V}β₃ using techniques well known to those skilled in the art. (See, e.g., Greenspan & Bona, 1989, FASEB J. 7(5):437-444; and Nissinoff. 1991, J. Immunol. 147(8):2429-2438). For example, antibodies of the invention which bind to and competitively inhibit the binding of Integrin α_{V}β₃ (as determined by assays well known in the art and disclosed supra) to its ligands can be used to generate anti-idiotypes that “mimic” Integrin α_{V}β₃ binding domains and, as a consequence, bind to and neutralize Integrin α_{V}β₃ and/or its ligands. Such neutralizing anti-idiotypes or Fab fragments of such anti-idiotypes can be used in therapeutic regimens to neutralize Integrin α_{V}β₃. The invention provides methods employing the use of polynucleotides comprising a nucleotide sequence encoding an antibody of the invention or a fragment thereof.

5.4.3 Polynucleotides Encoding an Antibody

The methods of the invention also encompass polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. Since the amino acid sequences of the antibodies are known, nucleotide sequences encoding these antibodies can be determined using methods well known in the art, i.e., nucleotide codons known to encode particular amino acids are assembled in such a way to generate a nucleic acid that encodes the antibody or fragment thereof of the invention. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to Integrin α_{V}β₃. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.4.4 Recombinant Expression of an Antibody

Recombinant expression of an antibody of the invention, derivative, analog or fragment thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies or fragments thereof which immunospecifically bind to Integrin α_{V}β₃ is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI 38, BT483, Hs578T, HTB2, BT20 and T47D, NS0 (a murine myeloma cell line that does not endogenously produce any immunoglobulin chains), CRL7030 and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.5 Peptides, Polypeptides and Fusion Proteins

That Immunospecifically Bind to Integrin a The present invention encompasses peptides, polypeptides and fusion proteins that immunospecifically bind to Integrin α_{V}β₃ for use as Integrin α_{V}β₃ antagonists in preventing, treating, managing or ameliorating cancer or one or more symptoms thereof. In particular, the present invention encompasses peptides, polypeptides and fusion proteins that immunospecifically bind to Integrin α_{V}β₃ expressed by cancer cells.

In a specific embodiment, a peptide, a polypeptide or a fusion protein that immunospecifically binds to Integrin α_{V}β₃ inhibits or reduces the interaction between Integrin α_{V}β₃ and its ligands by approximately 25%, 30%, 35%, 45%, 50%, 55%, 60%, 65%, 70%, 75%, 80%, 85%, 90%, 95%, or 98% in an in vivo or in vitro assay described herein or well-known to one of skill in the art. Examples of Integrin α_{V}β₃ ligands include, but are not limited to, vitronectin, osteopontin, bone sialoprotein, echistatin, RGD-containing peptides, and RGD mimetics. (See e.g., Dresner-Pollak et al., J. Cell Biochem. 56(3):323-30; Duong et al., Front. Biosci. 1(3):d757-68). In alternative embodiment, a peptide, a polypeptide or a fusion protein that immunospecifically binds to Integrin α_{V}β₃ does not significantly inhibit the interaction between Integrin α_{V}β₃ and its ligands in an in vivo or in vitro assay described herein or well-known to one of skill in the art.

In one embodiment, a peptide, a polypeptide or a fusion protein that immunospecifically binds to Integrin α_{V}β₃ comprises a bioactive molecule fused to the Fc domain of an immunoglobulin molecule or a fragment thereof. In another embodiment, a peptide, a polypeptide or a fusion protein that immunospecifically binds to Integrin α_{V}β₃ comprises a bioactive molecule fused to the CH2 and/or CH3 region of the Fc domain of an immunoglobulin molecule. In yet another embodiment, a peptide, a polypeptide or a fusion protein that immunospecifically binds to Integrin αᵥβ₃ comprises a bioactive molecule fused to the CH2, CH3, and hinge regions of the Fc domain of an immunoglobulin molecule. In accordance with these embodiments, the bioactive molecule immunospecifically binds to Integrin α_{V}β₃. Bioactive molecules that immunospecifically bind to Integrin α_{V}β₃ include, but are not limited to, peptides, polypeptides, proteins, small molecules, mimetic agents, synthetic drugs, inorganic molecules, and organic molecules. Preferably, a bioactive molecule that immunospecifically binds to Integrin α_{V}β₃ is a polypeptide comprising at least 5, preferably at least 10, at least 20, at least 30, at least 40, at least 50, at least 60, at least 70, at least 80, at least 90 or at least 100 contiguous amino acid residues, and is heterologous to the amino acid sequence of the Fc domain of an immunoglobulin molecule or a fragment thereof.

In a specific embodiment, a peptide, a polypeptide or a fusion protein that immunospecifically binds to Integrin αᵥβ₃ comprises an Integrin α_{V}β₃ ligand or a fragment thereof which immunospecifically binds to an Integrin αᵥβ₃ fused to the Fc domain of an immunoglobulin molecule or a fragment thereof. Examples of Integrin α_{V}β₃ ligands include, but are not limited to, vitronectin, osteopontin, bone sialoprotein, echistatin, RGD-containing peptides, and RGD mimetics. (See e.g., Dresner-Pollak et al., J. Cell Biochem. 56(3):323-30; Duong et al., Front. Biosci. 1(3):d757-68). In another embodiment, a peptide, a polypeptide or a fusion protein that immunospecifically binds to Integrin αᵥβ₃ comprises an Integrin αᵥβ₃ ligand or a fragment thereof which immunospecifically binds to Integrin α_{V}β₃ fused to the CH2 and/or CH3 region of the Fc domain of an immunoglobulin molecule. In another embodiment, a peptide, a polypeptide or a fusion protein that immunospecifically binds to Integrin αᵥβ₃ comprises an Integrin α_{V}β₃ ligand or a fragment thereof which immunospecifically binds to Integrin α_{V}β₃ fused to the CH2, CH3, and hinge regions of the Fc domain of an immunoglobulin molecule.

In another embodiment, a peptide, a polypeptide or a fusion protein that immunospecifically binds to Integrin α_{V}β₃ comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an Integrin αᵥβ₃ ligand or a fragment thereof fused to the Fc domain of an immunoglobulin molecule or a fragment thereof. In another embodiment, a peptide, a polypeptide or a fusion protein that immunospecifically binds to Integrin α_{V}β₃ comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an Integrin αᵥβ₃ ligand or a fragment thereof fused to the CH2 and/or CH3 region of the Fc domain of an immunoglobulin molecule. In another embodiment, a peptide, a polypeptide or a fusion protein that immunospecifically binds to Integrin α_{V}β₃ comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an Integrin α_{V}β₃ ligand or a fragment thereof fused to the CH2, CH3, and hinge regions of the Fc domain of an immunoglobulin molecule.

The present invention provides peptides, polypeptides or fusion proteins that immunospecifically bind to Integrin α_{V}β₃ comprising the Fc domain of an immunoglobulin molecule or a fragment thereof fused to a polypeptide encoded by a nucleic acid molecule that hybridizes to the nucleotide sequence encoding an Integrin α_{V}β₃ ligand or a fragment thereof.

In a specific embodiment, a peptide, a polypeptide or a fusion protein that immunospecifically binds to Integrin α_{V}β₃ comprises the Fc domain of an immunoglobulin molecule or a fragment thereof fused to a polypeptide encoded by a nucleic acid molecule that hybridizes to the nucleotide sequence encoding an Integrin α_{V}β₃ ligand or a fragment thereof under stringent conditions, e.g., hybridization to filter-bound DNA in 6× sodium chloride/sodium citrate (SSC) at about 45 [symbol] followed by one or more washes in 0.2×SSC/0.1% SDS at about 50-65 [symbol] under highly stringent conditions, e.g., hybridization to filter-bound nucleic acid in 6×SSC at about 45 [symbol] followed by one or more washes in 0.1×SSC/0.2% SDS at about 68 [symbol] or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

5.5.1 Peptide, Polypeptide and Fusion Protein Conjugate

The present invention also encompasses peptides, polypeptides and fusion proteins, which immunospecifically bind to Integrin α_{V}β₃, fused to marker sequences, such as but not limited to, a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses peptides, polypeptides and fusion proteins that immunospecifically bind to Integrin α_{V}β₃ conjugated to a therapeutic moiety. A peptide, a polypeptide or a fusion protein that immunospecifically binds to Integrin α_{V}β₃ may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Other agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a peptide, a polypeptide or a fusion protein that immunospecifically binds to Integrin α_{V}β₃ may be conjugated to a therapeutic moiety or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, IFN-α, IFN-β, NGF, PDGF, TPA, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., IL-1, IL-2, IL-6, IL-10, GM-CSF, and G-CSF), or a growth factor (e.g., GH).

5.5.2 Methods of Producing Polypeptides and Fusion Proteins

Peptides, polypeptides, proteins and fusion proteins can be produced by standard recombinant DNA techniques or by protein synthetic techniques, e.g., by use of a peptide synthesizer. For example, a nucleic acid molecule encoding a peptide, polypeptide, protein or a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992). Moreover, a nucleic acid encoding a bioactive molecule can be cloned into an expression vector containing the Fc domain or a fragment thereof such that the bioactive molecule is linked in-frame to the Fc domain or Fc domain fragment.

Methods for fusing or conjugating polypeptides to the constant regions of antibodies are known in the art. See, e.g., U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; International Publication Nos. WO 91/06570, WO 96/04388, WO 96/22024, WO 97/34631, and WO 99/04813; Ashkenazi et al., 1991, Proc. Natl. Acad. Sci. USA 88: 10535-10539; Traunecker et al., 1988, Nature, 331:84-86; Zheng et al., 1995, J. Immunol. 154:5590-5600; and Vil et al., 1992, Proc. Natl. Acad. Sci. USA 89:11337-11341, which are incorporated herein by reference in their entireties.

The nucleotide sequences encoding a bioactive molecule and an Fc domain or fragment thereof may be an be obtained from any information available to those of skill in the art (i.e., from Genbank, the literature, or by routine cloning). The nucleotide sequences encoding Integrin ligands may be obtained from any available information, e.g., from Genbank, the literature or by routine cloning. See, e.g., Xiong et al., Science, 12; 294(5541):339-45 (2001). The nucleotide sequence coding for a polypeptide a fusion protein can be inserted into an appropriate expression vector, i.e., a vector which contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The expression of a peptide, polypeptide, protein or a fusion protein may be controlled by any promoter or enhancer element known in the art. Promoters which may be used to control the expression of the gene encoding fusion protein include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the O-lactamase promoter (Villa-Kamaroff et al., 1978, Proc. Natl. Acad. Sci. U.S.A. 75:3727-3731), or the tac promoter (DeBoer et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Orhitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Corn. 253:818-823); glial fibrillary acidic protein (GFAP) promoter which is active in astrocytes (Gomes et al., 1999, Braz J Med Biol Res 32(5):619-63-1; Morelli et al., 1999, Gen. Virol. 80:571-83) and gonadotropic releasing hormone gene control region which is active in the hypothalamus (Mason et al., 1986, Science 234:1372-1378).

In a specific embodiment, the expression of a peptide, polypeptide, protein or a fusion protein is regulated by a constitutive promoter. In another embodiment, the expression of a peptide, polypeptide, protein or a fusion protein is regulated by an inducible promoter. In another embodiment, the expression of a peptide, polypeptide, protein or a fusion-protein is regulated by a tissue-specific promoter.

In a specific embodiment, a vector is used that comprises a promoter operably linked to a peptide-, polypeptide-, protein- or a fusion protein-encoding nucleic acid, one or more origins of replication, and, optionally, one or more selectable markers (e.g., an antibiotic resistance gene).

In mammalian host cells, a number of viral-based expression systems may be utilized. In cases where an adenovirus is used as an expression vector, the polypeptide or fusion protein coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted fusion protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a peptide, polypeptide, protein or a fusion protein can be identified by three general approaches: (a) nucleic acid hybridization, (b) presence or absence of “marker” gene functions, and (c) expression of inserted sequences. In the first approach, the presence of a gene encoding a peptide, polypeptide, protein or a fusion protein in an expression vector can be detected by nucleic acid hybridization using probes comprising sequences that are homologous to an inserted gene encoding the peptide, polypeptide, protein or the fusion protein, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a polypeptide or a fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (e.g., fusion protein) expressed by the recombinant. Such assays can be based, for example, on the physical or functional properties of the fusion protein in in vitro assay systems, e.g., binding with anti-bioactive molecule antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, MDCK, 293, 3T3, WI38, NS0, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl. Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express a polypeptide or a fusion protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a polypeptide or a fusion protein that immunospecifically binds to Integrin α_{V}β₃. Such engineered cell lines may be particularly useful in screening and evaluation of compounds that affect the activity of a polypeptide or a fusion protein that immunospecifically binds to Integrin α_{V}β₃.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt-cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

Once a peptide, polypeptide, protein or a fusion protein of the invention has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

5.6 Other Prophylactic/Therapeutic Agents

According to the invention, cancer or one or more symptoms thereof may be prevented, treated, managed or ameliorated by the administration of an antagonist of Integrin α_{V}β₃ in combination with the administration of one or more therapies such as, but not limited to, chemotherapies, radiation therapies, hormonal therapies, and/or biological therapies/immunotherapies.

In a specific embodiment, the methods of the invention encompass the administration of one or more angiogenesis antagonists such as but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates (such as but are not limited to, alendronate, clodronate, etidronate, ibandronate, pamidronate, risedronate, tiludronate, and zoledronate).

In a specific embodiment, the methods of the invention encompass the administration of one or more immunomodulatory agents, such as but not limited to, chemotherapeutic agents and non-chemotherapeutic immunomodulatory agents. Non-limiting examples of chemotherapeutic agents include methotrexate, cyclosporin A, leflunomide, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologs, and cytoxan. Examples of non-chemotherapeutic immunomodulatory agents include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), anti-IL-12 antibodies and anti-IL-23 antibodies)); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, IL-23, TNF-Q TNF-0, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies and anti-IFN-γ antibodies), and antibodies that immunospecifically bind to tumor-associated antigens (e.g., Herceptin®). In certain embodiments, an immunomodulatory agent is an immunomodulatory agent other than a chemotherapeutic agent. In other embodiments an immunomodulatory agent is an immunomodulatory agent other than a cytokine or hemapoietic such as IL-1, IL-2, IL-4, IL-12, IL-15, TNF, IFN-α, IFN-β, IFN-γ, M-CSF, G-CSF, IL-3 or erythropoietin. In yet other embodiments, an immunomodulatory agent is an agent other than a chemotherapeutic agent and a cytokine or hemapoietic factor.

In a specific embodiment, the methods of the invention encompass the administration of one or more anti-inflammatory agents, such as but not limited to, non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODIN™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In another specific embodiment, the methods of the invention encompass the administration of one or more antiviral agents (e.g., amantadine, ribavirin, rimantadine, acyclovir, famciclovir, foscarnet, ganciclovir, trifluridine, vidarabine, didanosine, stavudine, zalcitabine, zidovudine, interferon), antibiotics (e.g., dactinomycin (formerly actinomycin), bleomycin, mithramycin, and anthramycin (AMC)), anti-emetics (e.g., alprazolam, dexamethoasone, domperidone, dronabinol, droperidol, granisetron, haloperidol, haloperidol, iorazepam, methylprednisolone, metoclopramide, nabilone, ondansetron, prochlorperazine), anti-fungal agents (e.g., amphotericin, clotrimazole, econazole, fluconazole, flucytosine, griseofulvin, itraconazole, ketoconazole, miconazole and nystatin), anti-parasite agents (e.g., dehydroemetine, diloxanide furoate, emetine, mefloquine, melarsoprol, metronidazole, nifurtimox, paromomycin, pentabidine, pentamidine isethionate, primaquine, quinacrine, quinidine) or a combination thereof.

Specific examples of anti-cancer agents that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1 interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix: anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didernin B; didox; diethylnorspennine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-CoA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sd±1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; Vitaxin®; vorozole; zanoterone; zeniplatin; zilascorb; and zinostatin stimalamer. Preferred additional anti-cancer drugs are 5-fluorouracil and leucovorin. These two agents are particularly useful when used in methods employing thalidomide and a topoisomerase inhibitor. In specific embodiments, a anti-cancer agent is not a chemotherapeutic agent.

In more particular embodiments, the present invention also comprises the administration of an antagonist of Integrin α_{V}β₃ in combination with the administration of one or more therapies such as, but not limited to anti-cancer agents such as those disclosed in Table 3, preferably for the treatment of breast, ovary, melanoma, prostate, colon and lung cancers as described above. When used in a combination therapy, the dosages and/or the frequency of administration listed in Table 3 may be decreased.

TABLE 3

| Therapeutic | | | |
|---|---|---|---|
| Agent | Dose/Administration/Formulation | | |
| doxorubicin | Intravenous | 60-75 mg/m² on Day 1 | 21 day intervals |
| hydrochloride | | | |
| (Adriamycin | | | |
| RDF ® and | | | |
| Adriamycin PFS ® | | | |
| epirubicin | Intravenous | 100-120 mg/m² on Day 1 of each | 3-4 week cycles |
| hydrochloride | | cycle or | |
| (Ellence ™) | | divided equally and given on Days | |
| | | 1-8 of the cycle | |
| fluorousacil | Intravenous | How supplied: | |
| | | 5 mL and 10 mL vials (containing | |
| | | 250 and 500 mg flourouracil | |
| | | respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for |
| (Taxol ®) | | | 4 courses (administered |
| | | | sequentially to doxorubicin- |
| | | | containing combination |
| | | | chemotherapy) |
| tamoxifen citrate | Oral | 20-40 mg | Daily |
| (Nolvadex ®) | (tablet) | Dosages greater than 20 mg should | |
| | | be given in divided doses (morning | |
| | | and evening) | |
| leucovorin calcium | Intravenous or | How supplied: | Dosage is unclear from text. |
| for injection | intramuscular | 350 mg vial | PDR 3610 |
| | injection | | |
| luprolide acetate | Single | 1 mg (0.2 mL or 20 unit mark) | Once a day |
| (Lupron ®) | subcutaneous | | |
| | injection | | |
| flutamide | Oral (capsule) | 250 mg | 3 times a day at 8 hour intervals |
| (Eulexin ®) | | (capsules contain 125 mg | (total daily dosage 750 mg) |
| | | flutamide each) | |
| nilutamide | Oral | 300 mg or 150 mg | 300 mg once a day for 30 days |
| (Nilandron ®) | (tablet) | (tablets contain 50 or 150 mg | followed by 150 mg once a day |
| | | nilutamide each) | |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/mL | |
| ketoconazole | Cream | 2% cream applied once or twice | |
| (Nizoral ®) | | daily depending on symptoms | |
| prednisone | Oral | Initial dosage may vary from 5 mg | |
| | (tablet) | to 60 mg per day depending on the | |
| | | specific disease entity being | |
| | | treated. | |
| estramustine | Oral | 14 mg/kg of body weight (i.e. one | Daily given in 3 or 4 divided |
| phosphate sodium | (capsule) | 140 mg capsule for each 10 kg or | doses |
| (Emcyt ®) | | 22 lb of body weight) | |
| etoposide or | Intravenous | 5 mL of 20 mg/mL solution (100 mg) | |
| VP-16 | | | |
| dacarbazine | Intravenous | 2-4.5 mg/kg | Once a day for 10 days. |
| (DTIC-Dome ®) | | | May be repeated at 4 week |
| | | | intervals |
| polifeprosan 20 with | wafer placed in | 8 wafers, each containing 7.7 mg | |
| carmustine implant | resection | of carmustine, for a total of 61.6 | |
| (BCNU) | cavity | mg, if size and shape of resection | |
| (nitrosourea) | | cavity allows | |
| (Gliadel ®) | | | |
| cisplatin | Injection | [n/a in PDR 861] | |
| | | How supplied: | |
| | | solution of 1 mg/mL in multi-dose | |
| | | vials of 50 mL and 100 mL | |
| mitomycin | Injection | supplied in 5 mg and 20 mg vials | |
| | | (containing 5 mg and 20 mg mitomycin) | |
| gemcitabine HCl | Intravenous | For NSCLC-2 schedules have | 4 week schedule- |
| (Gemzar ®) | | been investigated and the optimum | Days 1, 8 and 15 of each 28-day |
| | | schedule has not been determined | cycle. Cisplatin intravenously |
| | | 4 week schedule- | at 100 mg/m² on day 1 after the |
| | | administration intravenously at | infusion of Gemzar. |
| | | 1000 mg/m² over 30 minutes on 3 | 3 week schedule- |
| | | week schedule- | Days 1 and 8 of each 21 day |
| | | Gemzar administered | cycle. Cisplatin at dosage of |
| | | intravenously at 1250 mg/m² over | 100 mg/m² administered |
| | | 30 minutes | intravenously after |
| | | | administration of Gemzar on |
| | | | day 1. |
| carboplatin | Intravenous | Single agent therapy: | Every 4 weeks |
| (Paraplatin ®) | | 360 mg/m² I.V. on day 1 | |
| | | (infusion lasting 15 minutes or | |
| | | longer) | |
| | | Other dosage calculations: | |
| | | Combination therapy with | |
| | | cyclophosphamide, Dose | |
| | | adjustment recommendations, | |
| | | Formula dosing, etc. | |
| ifosamide | Intravenous | 1.2 g/m² daily | 5 consecutive days |
| (Ifex ®) | | | Repeat every 3 weeks or after |
| | | | recovery from hematologic |
| | | | toxicity |
| topotecan | Intravenous | 1.5 mg/m² by intravenous infusion | 5 consecutive days, starting on |
| hydrochloride | | over 30 minutes daily | day 1 of 21 day course |
| (Hycamtin ®) | | | |
| Bisphosphonates | | | |
| Pamidronate | Intravenous | 60 mg or 90 mg single infusion | |
| | | over 4-24 hours to correct | |
| | | hypercalcemia in cancer patients | |
| Alendronate | Oral | 5 mg/d daily for 2 years and then | |
| | | 10 mg/d for 9 month to prevent or | |
| | | control bone resorption. | |
| Risedronate | Oral, take with | 5.0 mg to prevent or control bone | |
| | 6-8 oz water. | resorption. | |
| Lovastatin | Oral | 10-80 mg/day in single or two | |
| (Mevacor ™) | | divided dose. | |

The invention also encompasses administration of Integrin α_{V}β₃ antagonists in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radiaoactive source is placed inside the body close to cancer cells or a tumor mass.

Cancer therapies and their dosages, routes of administration and recommended usage are known in the art and have been described in such literature as the Physician's Desk Reference (56^{th} ed., 2002).

5.7 Biological Assays

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The effect of one or more doses of one or more antagonists of Integrin α_{V}β₃ on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS.

The effect of one or more antagonists of Integrin αᵥβ₃ on blockage of Integrin αᵥβ₃ activity and/or the plasma concentration of Integrin αᵥβ₃ can be assayed by any technique known in the art that measuring the activity and/or expression of Integrin αᵥβ₃, including but not limited to, Western blot, Northern blot, RNase protection assays, enzymatic activity assays, in situ hybridization, immunohistochemistry, and immunocytochemistry. In any of these assays the probe to be used is specific to αᵥβ₃ or its ligand whose expression is to be investigated.

The binding specificity, affinity and functional activity of an antagonist of Integrin αᵥβ₃ of the invention can be characterized in various in vitro binding and cell adhesion assays known in the art, including but limited to, those that are disclosed in International Publication Nos. WO 00/78815 and WO 02/070007, U.S. Pat. No. 6,248,326, U.S. Pat. No. 6,472,403, Pecheur et al., 2002, FASEB J. 16(10):1266-1268; Ahmed et al., The Journal of Histochemistry & Cytochemistry 50:1371-1379 (2002), all of which are incorporated herein by reference.

The binding specificity of an antagonist of Integrin αᵥβ₃ of the invention can be assessed by measuring binding to Integrin αᵥβ₃ and its crossreactivity to other αᵥ- or β₃-containing integrins. Specifically, binding specificity can be assessed by measuring binding to α_{11b}β₃, the major Integrin expressed on platelets, and to Integrin αᵥβ₅, an Integrin found prevalent on endothelial cells and connective tissue cell types. Briefly, to determine crossreactivity, integrins are coated onto an ELISA plate and a series of antibody dilutions are measured for antibody binding activity against Integrin αᵥβ₃ and the other integrins. The integrins αᵥβ₃ and αᵥβ₅ can be isolated by know techniques in the art, e.g., by affinity chromatography as described in Cheresh, Proc. Natl. Acad. Sci. USA 84:6471-6475 (1987), and Cheresh and Spiro, J. Biol. Chem. 262:17703-17711 (1987). In a specific embodiment, an anti-Integrin αᵥβ₃ antibody affinity column is used to isolate Integrin αᵥβ₃ from an octylglucoside human placental lysate, whereas an anti-a affinity column is used to isolate

Integrin αᵥβ₅ from the Integrin αᵥβ₃ depleted column flow through. Antibody binding activity is assessed by ELISA using a goat anti-human IgG-alkaline phosphatase conjugate. A purified human IgG, antibody can be used as a control.

In another embodiment, the binding affinity and specificity are assessed in a competitive binding assay with the parental anti-Integrin αᵥβ₃ antibody against Integrin αᵥβ₃. Competitive binding is measured in an ELISA assay. Binding of the antibody is determined in the presence of increasing concentrations of antibody competitor. Alternatively, the control competitor antibody is again a human IgG₁.

In another embodiment, binding affinity and specificity are assessed by measuring the inhibitory activity of an antagonist of Integrin αᵥβ₃ on Integrin αᵥβ₃ binding to fibrinogen. Briefly, αᵥβ₃ is plated onto ELISA plates. Inhibitory activity of the antagonist of Integrin αᵥβ₃ is determined by measuring the amount of bound biotinylated fibrinogen in the presence of increasing concentrations of antagonist or control antibody. Streptavidin alkaline phosphatase is used to detect the bound fibrinogen.

In another embodiment, the specificity of the antagonist binding is assessed by the inhibition of Integrin αᵥβ₃ binding in cell adhesion assays. Endothelial cell adhesion events are an important component in the angiogenic process and inhibition of Integrin αᵥβ₃ is known to reduce the neovascularization of tumors and thereby reduce the rate of tumor growth. The inhibition of αᵥβ₃-mediated cell attachment by an Integrin αᵥβ₃ antagonist in these assays is indicative of the inhibitory activity expected when this antagonist is used in situ or in vivo. Briefly, Integrin αᵥβ₃-positive M21 melanoma cells grown in RPMI containing 10% FBS are used for these cell binding assays. Cells are released from the culture dish by trypsinization and re-suspended in adhesion buffer at a concentration of 4×10⁵ cells/ml. The antibody and the control antibody are diluted to the desired concentration in 250 μl adhesion buffer (10 mM Hepes, 2 mM MgCl₂, 2 mM CaCl₂, 0.2 mM MnCl₂, and 1% BSA in Hepes buffered saline at pH 7.4) and added to wells of a 48-well plate precoated with fibrinogen. Each well is coated with 200 μl fibrinogen at a concentration of 10 μg/ml for 1 hour at 37° C. For the assay, an equal volume of cells (250 μl) containing the antibody or isotype matched control antibody is added to each of the wells, mixed by gentle shaking and incubated for 20 minutes at 37° C. Unbound cells are removed by washing with adhesion buffer until no cells remained in control wells coated with BSA alone. Bound cells are visualized by staining with crystal violet which is subsequently extracted with 100 μl acetic acid (10%) and quantitated by determining the absorbance of the solubilized dye at 560 nm.

In another embodiment, the inhibitory activity of an antagonist of Integrin αᵥβ₃ is also tested in an endothelial cell migration assay. In this regard, the Transwell cell migration assay is used to assess the ability of an anti-Integrin αᵥβ₃ antibody to inhibit endothelial cell migration (Choi et al., J. Vascular Surg., 19:125-134 (1994) and Leavesly et al., J. Cell Biol., 121:163-170 (1993). Briefly, human umbilical vein endothelial cells in log phase and at low passage number are harvested by gentle trypsinization, wash and resuspend at a concentration of 2×10⁶ cells/ml in 37° C. HBS containing 1% BSA (20 mM Hepes, 150 mM NaCl, 1.8 mM MgCl₂, 1.8 mM CaCl₂, 5 mM KCl, and 5 mM glucose, pH 7.4). Antibodies are diluted to 10 μl/ml from stock solutions. Anti-Integrin αᵥβ₃ antibodies are added to cells in a 1:1 dilution (final concentration of antibodies=5 μg/ml; final concentration of cells=1×10⁶ cells/ml) and incubated on ice for 10-30 minutes. The cell/antagonist suspensions (200 μl to each compartment) are then added to the upper compartments of a Transwell cell culture chamber, the lower compartments of which had been coated with 0.5 ml of 10 μg/ml vitronectin (in HBS). Vitronectin serves as the chemoattractant for the endothelial cells. The chambers are placed at 37° C. for 4 hours to allow cell migrate to occur. Visualization of cell migration is performed by first removing the remaining cells in the upper compartment with a cotton swab. Cells that had migrated to the lower side of insert are stained with crystal violet for 30 minutes, followed by solubilization in acetic acid and the absorbance of the dye is measure at a wavelength of 550 nm. The amount of absorbance is directly proportional to the number of cells that have migrated from the upper to the lower chamber.

Additional examples of in vitro assays, e.g., Western blotting analysis, flow cytometric analysis, cell adhesion assay to cortical bone and extracellular matrix proteins, cell migration assay, cell invasion assay, and cell proliferation assay, can be found in Pecheur et al., 2002, FASEB J. 16(10):1266-1268, of which the entire text is incorporated herein by reference.

The anti-cancer activity of the therapies used in accordance with the present invention also can be determined by using various experimental animal models for the study of cancer such as the scid mouse model or transgenic mice where a mouse Integrin α_{V}β₃ is replaced with the human Integrin α_{V}β₃, nude mice with human xenografts, animal models wherein an antagonist of Integrin α_{V}β₃ recognizes the same target as Vitaxin®, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties. The following are some assays provided as examples and not by limitation.

Various animal models known in the art that are relevant to a particular cancer can be used, including but not limited to, those that are disclosed in International Publication No. WO 00/78815, U.S. Pat. No. 6,248,326, U.S. Pat. No. 6,472,403, Pecheur et al., 2002, FASEB J. 16(10):1266-1268; Ahmed et al., The Journal of Histochemistry & Cytochemistry 50:1371-1379 (2002), all of which are incorporated herein by reference.

In one embodiment, inhibition of tumor growth by an antagonist of Integrin α_{V}β₃ is tested in two animal models. The first model measures angiogenesis in the chick chorioallantoic membrane (CAM). This assay is a well recognized model for in vivo angiogenesis because the neovascularization of whole tissue is occurring. Specifically, the assay measures growth factor induced angiogenesis of chicken CAM vessels growing toward the growth factor-impregnated filter disk or into the tissue grown on the CAM. Inhibition of neovascularization is based on the amount and extent of new vessel growth or on the growth inhibition of tissue on the CAM. The assay has been described in detail by others and has been used to measure neovascularization as well as the neovascularization of tumor tissue (Ausprunk et al., Am. J. Pathol., 79:597-618 (1975); Ossonski et al., Cancer Res., 40:2300-2309 (1980); Brooks et al., Science, 264:569-571 (1994a) and Brooks et al., Cell, 79:1157-1164 (1994b). Briefly, for growth factor induced angiogenesis filter disks are punched from #1 Whatman Qualitative Circles using a skin biopsy punch. Disks are first sterilized by exposure to TV light and then saturated with varying concentrations of TNF-α of HBSS as a negative control (for at least 1 hour) under sterile conditions. Angiogenesis is induced by placing the saturated filter disks on the CAMs. Inhibition of angiogenesis is performed by treating the embryos with various amounts of an antagonist of Integrin αᵥβ₃ and controls (antibody or purified human IgG₁). The treatments are performed by intravenous injection approximately 24 hours after disk placement. After 48 hours, CAMs are dissected and angiogenesis is scored on a scale of 1-4. HBSS saturated filter disks are used as the negative control, representing angiogenesis that may occur in response to tissue injury in preparing CAMs, and, values for these CAMs are subtracted out as background. Purified human IgG, can be used as the negative control for injections.

In addition to the above described CAM assay using growth factor-induced neovascularization, additional assays can be performed utilizing tumor-induced neovascularization. For these assays, angiogenesis is induced by transplanting of Integrin αᵥβ₃-negative tumor fragments into the CAMs. The use of Integrin αᵥβ₃-negative tumor fragments ensures that any inhibition of tumor growth is due to the inhibition of αᵥβ₃-mediated neovascularization by CAM-derived endothelial cells and not to adhesion events mediated by Integrin αᵥβ₃ present on the tumor cells. Inhibition of tumor growth is assessed by placing a single cell suspension of FG (8×10⁶ cells, pancreatic carcinoma) and Hep-3 cells (5×10⁵ cells, laryngeal carcinoma) onto CAMs in 30 μl. One week later, tumors are removed and cut into approximately 50 mg fragments at which time they are placed onto new CAMs. After 24 hours of this second placement, embryos are injected intravenously with an anti-Integrin α_{V}β₃ antibody or human IgG, as a negative control. The tumors are allowed to grow for about 7 days following which they are removed and weighed.

In a second animal model, the inhibition of Vx2 carcinoma cells in rabbits is used as a measure of inhibitory effect on tumors of an antagonist of Integrin αᵥβ₃ The Vx2 carcinoma is a transplantable carcinoma derived from a Shope virus-induced papilloma. It was first described in 1940 and has since been used extensively in studies on tumor invasion, tumor-host interactions and angiogenesis. The Vx2 carcinoma is fibrotic in nature, highly aggressive, and exhibits features of an anaplastic type carcinoma. Propagation of Vx2 tumor is accomplished through serial transplantation in donor rabbits. Following subcutaneous transplantation, it has been reported that after an initial inflammatory reaction, host repair mechanisms set in between days 2 and 4. This repair mechanism is characterized by the formation of new connective tissue and the production of new capillaries. The newly formed capillaries are restricted to the repair zone at day 4, however, by day 8 they have extended to the outer region of the tumor. These characteristics and the pharmacokinetics of an antagonist of Integrin α_{V}β₃ in rabbits can be used to determine initial doses and scheduling of treatments for these experiments.

Growth of Vx2 tumors in the above animal model is used to study the effect of an antagonist of Integrin αᵥβ₃ after early administration on primary tumor growth in rabbits implanted subcutaneously with Vx2 carcinoma. Briefly, Vx2 tumors (50 mg) are transplanted into the inner thigh of rabbits through an incision between the skin and muscle. Measurements of the primary tumor are taken throughout the experiment through day 25.

In another embodiment, BALB/c nu/nu mice are used as animal models to study cancer associated with aberrant bone metabolism and/or aberrant angiogenesis. Different cell lines (e.g., CHO, or a type of cancer cells such as breast cancer cells) expressing αᵥβ₃ in various forms can be injected intravenously into the nude mice. See Pecheur et al., supra. For example, CHO cells are transfected with various cDNA constructs of αᵥβ₃ (e.g., wild-type, mutated forms) and injected intravenously into nude mice. The effects of 9,0 (with various level of activity because of the mutations) and antagonists of Integrin αᵥβ₃ on bone metastases can be assessed by, e.g., radiograph, histological examination of bone tissue or statistical analysis.

In another embodiment, animals (healthy or previously constructed animal models) in space environment (e.g., space shuttle) can be used to assess an antagonist of Integrin αᵥβ₃ of the invention. Since astronauts in long space flights have been shown to lose bone density in a way that is similar to osteoporosis patient, but ten times faster than in people who have the advantage of Earth's gravity (see BioWorld Today, 14:13, Jan. 21, 2003), animals in space environment are ideal osteoporosis model for determining the effects of an antagonist of Integrin αᵥβ₃ of the invention on cancer related to aberrant bone metabolism and/or aberrant angiogenesis.

In another embodiment, SCID mice with subcutaneously implanted human bone fragments (SCID-human-bone model) are used as an animal model to assess the effects of an antagonist of Integrin αᵥβ₃ of the invention on diseases associated with aberrant bone metabolism and/or aberrant angiogenesis. For examples, cancer cells (e.g., human prostate cancer cells) are injected directly into human bone fragments in the animal model. At the same time, antibody treatment is initiated. The effects of an antagonist of Integrin αᵥβ₃ of the invention on bone metastases or angiogenesis can be assessed by comparing to a control group. See Nemeth et al., Clinical & Experimental Metastasis, 19 (Supp. 1):47 (2002).

### Demonstration of Therapeutic Utility

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed or angiogenesis assays. A lower level of proliferation or survival of the contacted cells indicates that the therapeutic agent is effective to treat the condition in the patient. Alternatively, instead of culturing cells from a patient, therapeutic agents and methods may be screened using cells of a tumor or malignant cell line, osteoclasts, endothelial cells or an endothelial cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc.

Prophylactic or therapeutic agents can be tested in suitable animal model systems prior to testing in humans, including but not limited to in rats, mice, chicken, cows, monkeys, rabbits, hamsters, etc.

The principle animal models for known in the art and widely used are known and described in the art as described above.

Further, any assays known to those skilled in the art can be used to evaluate the prophylactic and/or therapeutic utility of the combinatorial therapies disclosed herein for treatment or prevention of cancer.

5.8 Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of an Integrin α_{V}β₃ antagonist (preferably an antibody or fragment thereof that immunospecifically binds to integrin αᵥβ₃, and more preferably Vitaxin® or an antigen-binding fragment thereof) and/or an anti-cancer agent, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer an Integrin α_{V}β₃ antagonist or the combination of an Integrin α_{V}β₃ antagonist and a prophylactic agent or therapeutic agent useful for preventing or treating cancer, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural, intratumoral, intra-synovial, and mucosal (e.g., intranasal and oral routes). In a specific embodiment, prophylactic or therapeutic agents of the invention are administered intramuscularly, intravenously, intratumorally, orally, intra-synovially, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, polymers, Tissuel®, or fibers.

In yet another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

5.8.1 Formulations

Pharmaceutical compositions for use in accordance with the present invention may be formulated in conventional manner using one or more physiologically acceptable carriers or excipients.

Thus, the antagonists of Integrin α_{V}β₃ or other anti-cancer agents and their physiologically acceptable salts and solvates may be formulated for administration by inhalation or insufflation (either through the mouth or the nose) or oral, parenteral or mucosol (such as buccal, vaginal, rectal, sublingual) administration. In a preferred embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachette indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various chemotherapeutic, biological/immunotherapeutic and hormonal therapeutic agents are known in the art and often described in the Physician's Desk Reference, 56^{th} ed. (2002). For instance, in certain specific embodiments of the invention, the therapeutic agents of the invention can be formulated and supplied as provided in Table 1.

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments of the invention, Vitaxin® is formulated at 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 ml/mL, 225 mg/mL, 250 mg/mL, 275 mg/mL and 300 mg/mL for intravenous injections and at 5 mg/mL, 10 mg/mL, 25 mg/mL, 50 mg/mL, 75 mg/mL, 100 mg/mL, 125 mg/mL, 150 mg/mL, 175 mg/mL, 200 ml/mL, 225 mg/mL, 250 mg/mL, 275 mg/mL and 300 mg/mL for intravenous injections or repeated subcutaneous administration.

The compositions may, if-desired, be presented in a pack or dispenser device that may contain one or more unit dosage forms containing the active ingredient. The pack may for example comprise metal or plastic foil, such as a blister pack. The pack or dispenser device may be accompanied by instructions for administration.

In certain preferred embodiments, the pack or dispenser contains one or more unit dosage forms containing no more than 5 mg/mL Vitaxin® and no more than the recommended dosage formulation as determined in the Physician's Desk Reference (56^{th} ed. 2002, herein incorporated by reference in its entirety) for a particular cancer therapy.

5.8.2 Dosages

The amount of the composition of the invention which will be effective in the treatment, prevention, management or amelioration of cancer or one or more symptoms thereof can be determined by standard research techniques. For example, the dosage of the composition which will be effective in the treatment, prevention, management, or amelioration of cancer or one or more symptoms thereof can be determined by administering the composition to an animal model such as, e.g., the animal models disclosed herein or known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems.

For peptides, polypeptides, proteins, fusion proteins, and antibodies, the dosage administered to a patient is typically 0.01 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

In a preferred embodiment, the dose of an antibody or antibody fragment that immunospecifically binds to Integrin α_{V}β₃ (e.g., Vitaxin® or an antigen-binding fragment thereof) is 0.1 to 10 mg/kg/week, preferably 1 to 9 mg/kg/week, more preferably 2 to 8 mg/week, even more preferably 3 to 7 mg/kg/week, and most preferably 4 to 6 mg/kg/week. In another embodiment, a subject, preferably a human, is administered one or more doses of a prophylactically or therapeutically effective amount of an antibody or antibody fragment that immunospecifically binds to Integrin α_{V}β₃ (e.g., Vitaxin® or an antigen-binding fragment thereof), wherein the dose of a prophylactically or therapeutically effective amount of the antibody or antibody fragment in the liquid formulation of the invention administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject, preferably a human, is administered one or more doses of a prophylactically or therapeutically effective amount of an antibody or antibody fragment that immunospecifically binds to Integrin α_{V}β₃ (e.g., VITAXIN® or an antigen-binding fragment thereof), wherein the dose of a prophylactically or therapeutically effective amount of the antibody or antibody fragment in the liquid formulation of the invention administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In specific embodiments, an antibody or antibody fragment that immunospecifically binds to integrin α_{V}β₃ (e.g., VITAXIN® or an antigen-binding fragment thereof) is administered in a dosing regimen that maintains the plasma concentration of the antibody at a desirable level (e.g., about 0.1 to about 100 μg/ml), which continuously blocks the integrin α_{V}β₃ activity. In a specific embodiment, the dosage administered to a patient is between 0.1 mg/kg and 20 mg/kg of the patient's body weight, more preferably 1 mg/kg to 10 mg/kg of the patient's body weight.

In another embodiment, a subject, preferably a human, is administered one or more doses of a prophylactically or therapeutically effective amount of an antibody or antibody fragment that immunospecifically binds to integrin α_{V}β₃ (e.g., VITAXIN® or a fragment thereof), wherein the dose of a prophylactically or therapeutically effective amount of the antibody or antibody fragment in the liquid formulation of the invention administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject, preferably a human, is administered one or more doses of a prophylactically or therapeutically effective amount of an antibody or antibody fragment that immunospecifically binds to integrin α_{V}β₃ (e.g., VITAXIN® or a fragment thereof), wherein the dose of a prophylactically or therapeutically effective amount of the antibody or antibody fragment in the liquid formulation of the invention administered to said subject is decreased by, e.g., 0.01 μg/kg, 0.02 μg/kg, 0.04 μg/kg, 0.05 μg/kg, 0.06 μg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 3.0 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For other cancer therapeutic agents administered to a patient, the typical doses of various cancer therapeutics known in the art are provided in Table 3. The invention provides for any method of administrating lower doses of known prophylactic or therapeutic agents than previously thought to be effective for the prevention, treatment, management or amelioration of cancer or one or more symptoms thereof. Preferably, lower doses of known anti-cancer therapies are administered in combination with lower doses of Integrin α_{V}β₃ antagonists.

The dosages of prophylactic or therapeutically agents are described in the Physicians' Desk Reference (56^{th} ed., 2002).

5.9 Kits

The invention provides a pharmaceutical pack or kit comprising one or more containers filled with an Integrin α_{V}β₃ antagonist. The pharmaceutical pack or kit may further comprises one or more other prophylactic or therapeutic agents useful for the treatment of a cancer. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an Integrin α_{V}β₃ antagonist, in one or more containers. The kit may further comprises one or more other prophylactic or therapeutic agents useful for the treatment of cancer, in one or more containers. Preferably the Integrin αᵥβ₃ antagonist is Vitaxin® or an antigen-binding fragment thereof. In certain preferred embodiments, the other prophylactic or therapeutic agent is a chemotherapeutic. In certain preferred embodiments, the prophylactic or therapeutic agent is a biological or hormonal therapeutic. More preferably, the Integrin α_{V}β₃ antagonist is Vitaxin® or an antigen-binding fragment thereof and the other prophylactic or therapeutic agent is Taxol or Tamoxifen for the treatment of breast cancer or 5-FU (5-fluorouracil) and Leucovorin, optionally with Irnotecan for the treatment of colon cancer. In an alternative embodiment, a kit comprises an Integrin α_{V}β₃ antagonist and one or more other prophylactic or therapeutic agents useful for the treatment of cancer, in one container. Preferably the Integrin α_{V}β₃ antagonist is Vitaxin® or an antigen-binding fragment thereof. In certain preferred embodiments, the other prophylactic or therapeutic agent is a chemotherapeutic. In other preferred embodiments, the other prophylactic or therapeutic agent is a biological or hormonal therapeutic.

The invention also provides a pharmaceutical pack or kit comprising one or more containers filled with an Integrin α_{V}β₃ antagonist conjugated to another moiety, including but not limited to, a heterologous polypeptide, peptide or protein, a large molecule, a small molecule, a marker sequence, a diagnostic or detectable agent, a therapeutic agent, a radioactive metal ion, a second antibody, and a solid support. The pharmaceutical pack or kit may further comprises one or more other prophylactic or therapeutic agents useful for the treatment of a cancer, in one or more containers.

5.10 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material.

In a specific embodiment, an article of manufacture comprises packaging material and a pharmaceutical agent and instructions contained within said packaging material, wherein said pharmaceutical agent is an Integrin α_{V}β₃ antagonist (preferably an antibody or an antigen-binding fragment thereof, and more preferably, VITAXIN® or an antigen-binding fragment thereof) and a pharmaceutically acceptable carrier, and said instructions indicate a dosing regimen for preventing, treating or managing a subject with cancer. In another embodiment, an article of manufacture comprises packaging material and a pharmaceutical agent and instructions contained within said packaging material, wherein said pharmaceutical agent is an Integrin α_{V}β₃ antagonist (preferably an antibody or an antigen-binding fragment thereof, and more preferably, VITAXIN® or an antigen-binding fragment thereof), a prophylactic or therapeutic agent other than Integrin α_{V}β₃ antagonist and a pharmaceutically acceptable carrier, and said instructions indicate a dosing regimen for preventing, treating or managing a subject with a cancer. In another embodiment, an article of manufacture comprises packaging material and two pharmaceutical agents and instructions contained within said packaging material, wherein said first pharmaceutical agent is an Integrin α_{V}β₃ antagonist (preferably an antibody or an antigen-binding fragment thereof, and more preferably, VITAXIN® or an antigen-binding fragment thereof) and a pharmaceutically acceptable carrier and said second pharmaceutical agent is a prophylactic or therapeutic agent other than an Integrin α_{V}β₃ antagonist, and said instructions indicate a dosing regimen for preventing, treating or managing a subject with a cancer.

The present invention provides that the adverse effects that may be reduced or avoided by the methods of the invention are indicated in informational material enclosed in an article of manufacture for use in preventing, treating or ameliorating one or more symptoms associated with cancer. Adverse effects that may be reduced or avoided by the methods of the invention include but are not limited to vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation. Since some of the therapies may be immunosuppressive, prolonged immunosuppression may increase the risk of infection, including opportunistic infections. Prolonged and sustained immunosuppression may also result in an increased risk of developing certain types of cancer.

Further, the information material enclosed in an article of manufacture for use in preventing, treating or ameliorating cancer or one or more symptoms can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

The information material can also indicate that cytokine release syndrome is an acute clinical syndrome, temporally associated with the administration of certain antibodies. Cytokine release syndrome has been attributed to the release of cytokines by activated lymphocytes or monocytes. The clinical manifestations for cytokine release syndrome have ranged from a more frequently reported mild, self-limited, “flu-like” illness to a less frequently reported severe, life-threatening, shock-like reaction which may include serious cardiovascular, pulmonary and central nervous system manifestations. The syndrome typically begins approximately 30 to 60 minutes after administration (but may occur later) and may persist for several hours. The frequency and severity of this symptom complex is usually greatest with the first dose. With each successive dose, both the incidence and severity of the syndrome tend to diminish. Increasing the amount of a dose or resuming treatment after a hiatus may result in a reappearance of the syndrome. As mentioned above, the invention encompasses methods of treatment and prevention that avoid or reduce one or more of the adverse effects discussed herein.

5.11 Use of Integrin α_{V}β₃ Antagonists in the Analysis of Integrin α_{V}β₃-Expression

An Integrin α_{V}β₃ antagonist may be used to visualize the expression of Integrin C3 one cells or cell lines, and in tissue sections and biopsies. In certain embodiments, visualization of Integrin α_{V}β₃ in tissue sections and biopsies can be effected under various conditions as described in Example 3. In certain embodiments, the analysis of tissue samples and biopsies requires the use of frozen tissues. In preferred embodiments, the tissue samples and biopsies are prepared using standard methods for processing and paraffin embedding of tissue while allowing immunohistochemical staining of Integrin α_{V}β₃ in the resulting paraffin embedded tissue. Preferably, conditions and reagents used preserve the LM609 epitope on Integrin α_{V}β₃ and yet remain compatible with standard paraffin embedding procedures. In a specific preferred embodiment, visualization of the epitope on Integrin α_{V}β₃ that is recognized by LM609 is accomplished by use of tissue fixed in approximately 70% ethanol for, preferably 24 hours, but may be 12 to 36 hours prior to processing and paraffin embedding. Given the invention, such methods can facilitate the analysis of Integrin α_{V}β₃ expression in tissue samples from clinical trials, animal models, and biopsies.

The tissues analyzed in accordance with methods of the invention, in some embodiments, are tissues from cancer patients obtained during surgery. See Aimed et al., The Journal of Histochemistry & Cytochemistry 50:1371-1379 (2002). For example, the tissues from patient with ovary cancer presented for surgery are divided and frozen in cylinders of frozen section embedding medium (OCT) by immersion in isopentane cooled in dry ice. Frozen sections of the tissue are cut at 5 μm thickness and, if not used immediately stored at −20° C. For staining, sections are fixed in cold acetone for 15 minutes and held in Tris buffer (100 mM, pH 7.6). Endogenous peroxidase activity is removed using 3% H₂O₂ in methanol and endogenous biotin activity is blocked using a sequence of diluted egg white (5% in distilled water) and skim milk powder (5% in distilled water), all for 10 minutes. The sections are incubated for 1 hour with α_{V}β₃ Mab in Tris buffer (100 mM, pH 7.6). Antibody binding is amplified using biotin and streptavidin HRP for 15 minutes each and the complex is visualized using diaminobenzidine (DAB). Nuclei are lightly stained with Mayer's hematoxylin and the sections mounted and cover-slipped. An isotype IgG1, suitably diluted, is substituted for the antibody as a negative control. Sections are assessed microscopically for positive DAB staining by trained pathologists, and the degree of staining of αᵥβ₃ expression is scored in a blind fashion.

An Integrin α_{V}β₃ antagonist may be used to evaluating the metastatic potential of a cancer (e.g., lung cancer, breast cancer, prostate cancer, or ovarian cancer) by determining the expression and/or activity level of Integrin α_{V}β₃ one cells or cell lines, and in tissue sections and biopsies.

Labeled Integrin α_{V}β₃ antagonists (in particular, labeled anti-Integrin α_{V}β₃ antibodies) can be used for diagnostic purposes to detect, diagnose, or monitor cancer. Such diagnostic techniques are known in the art, including but not limited to, those disclosed in International Publication No. WO 01/58483, U.S. Pat. No. 6,248,326, Pecheur et al., 2002, FASEB J. 16(10):1266-1268, Almed et al., The Journal of Histochemistry & Cytochemistry 50:1371-1379 (2002), all of which are incorporated herein by reference. In a preferred embodiment, antibodies which immunospecifically bind to Integrin αᵥβ₃ covalently bound to IgG or IgM antibodies, or Integrin αᵥβ₃ covalently bound to a cell (e.g., a cancer cell) are used for diagnostic purposes to detect, diagnosis, or monitor a disease or disorder. The detection or diagnosis of cancer can be conducted utilizing an effective amount (i.e., an amount effective to be able to detect the expression of Integrin αᵥβ₃) of an Integrin αᵥβ₃ antagonist in an in vitro and/or in vivo assay using techniques well-known to one of skilled in the art. In a preferred embodiment, a cancer is detected in the subject, preferably a mammalian subject and most preferably a human subject utilizing an effective amount of an antibody of the invention in a standard imaging technique known to one of skilled in the art.

In a specific embodiment, the invention provides methods of detecting or diagnosing a disease or disorder, said methods comprising: a) administering to a subject an effective amount of a labeled Integrin αᵥβ₃ antagonist (preferably, an antibody or antibody fragment that immunospecifically binds to Integrin αᵥβ₃, and more preferably VITAXIN® or an antigen-binding fragment thereof); b) waiting for a time interval following the administering for permitting the labeled Integrin αᵥβ₃ antagonist to preferentially concentrate at any desired site, e.g., cancerous site, in the animal (and for unbound labeled Integrin αᵥβ₃ antagonist to be cleared to background level); c) determining background level; and d) detecting the labeled Integrin αᵥβ₃ antagonist in the subject, such that detection of labeled Integrin αᵥβ₃ antagonist above the background level indicates the presence of the disease.

In another embodiment, the invention provides methods of detecting or diagnosing a disease or disorder, said methods comprising: a) administering to a subject an effective amount of an Integrin αᵥβ₃ antagonist (preferably an antibody or antibody fragment that immunospecifically binds to Integrin αᵥβ₃ and more preferably, VITAXIN® or an antigen-binding fragment thereof); b) administering a second labeled agent, (e.g., an antibody or antibody fragment) that recognizes the Integrin αᵥβ₃ antagonist; c) waiting for a time interval following the administering for permitting the labeled agent to preferentially concentrate at any desired site, e.g., cancerous site, in the animal (and for unbound labeled agent to be cleared to background level); d) determining background level; and e) detecting the labeled agent in the subject, such that detection of labeled agent above the background level indicates the presence of the disease.

In yet another embodiment, the invention provides methods for the diagnosis or detection of cancer in a subject, said methods comprising imaging said subject at a time interval after administering to said subject an effective amount of a labeled Integrin αᵥβ₃ antagonist (in particular, an antibody or antibody fragment that immunospecifically binds to Integrin αᵥβ₃, preferably VITAXIN® or an antigen-binding fragment thereof), said time interval being sufficient to permit the labeled Integrin αᵥβ₃ antagonist to preferentially concentrate at a specific site, e.g., a cancerous site, in said subject, wherein detection of the labeled Integrin αᵥβ₃ antagonist localized at the site in the subject indicates the presence of the cancer. In a preferred embodiment, the cancer detected in vivo is a solid tumor cancer.

In some embodiments, monitoring of cancer is carried out by repeating the method for diagnosing the cancer, for example, one month after initial diagnosis, six month after initial diagnosis, and one year after initial diagnosis. In specific embodiments of the invention, the density of a tumor facilitates the detection of said tumor using anti-αᵥβ₃ antibodies in accordance with the method of the invention.

Presence of labeled Integrin αᵥβ₃ antagonist can be detected in the patient using methods known in the art for in vivo scanning. These methods depend upon the type of label used. Skilled artisans will be able to determine the appropriate method for detecting a particular label. Methods and devices that may be used in the diagnostic methods of the invention include but are not limited to: computed tomography (CT), whole body scan such as position emission tomography (PET), magnetic resonance imaging (MM), and sonography. In a specific embodiment, the Integrin αᵥβ₃ antagonist is labeled with a radioisotope and is detected in the patient using a radiation responsive surgical instrument (Thurston et al., U.S. Pat. No. 5,441,050). In another embodiment, the Integrin αᵥβ₃ antagonist is labeled with a fluorescent compound and is detected in the patient using a fluorescence responsive scanning instrument. In another embodiment, the Integrin αᵥβ₃ antagonist is labeled with a positron emitting metal and is detected in the patient using positron emission tomography. In yet another embodiment, the Integrin αᵥβ₃ antagonist is labeled with a paramagnetic label and is detected in a patient using magnetic resonance imaging (MRI).

### 6. EXAMPLE 1

### Treatment of Patients with Metastatic Breast Cancer

Certain embodiments of the invention, as well as certain novel and unexpected advantages of the invention, are illustrated by the following non-limiting example.

A study is designed to assess pharmacokinetics and safety of Vitaxin® in patients with metastatic breast cancer. Cancer patients currently receive Taxol or Taxotere. Patients currently receiving treatment are permitted to continue these medications.

Patients are administered a single IV dose of Vitaxin® and then, beginning 4 weeks later, are analyzed following administration of repeated weekly IV doses at the same dose over a period of 12 weeks. Vitaxin® safety and potential changes in disease activity over 26 weeks of IV dosing is also be assessed. Different groups of patients are treated and evaluated similarly but receive doses of 1 mg/kg, 2 mg/kg, 4 mg/kg, or 8 mg/kg.

Vitaxin® is formulated at 5 mg/mL and 10 mg/ml for IV injection. A formulation of 80 mg/mL is required for repeated subcutaneous administration. Vitaxin® is also formulated at 100 mg/mL for administration for the purposes of the study.

Changes are measured or determined by the progression of tumor growth.

Vitaxin® can be prepared and formulated in accordance with the disclosure of PCT Publication WO 00/78815 which is herein incorporated by reference in its entirety.

### EXAMPLE 2

### Determination of Binding Affinity for Integrin

α_{V}β₃ and Epitope Mapping of Integrin α_{V}β₃

Previous attempts to model the effects of Vitaxin® in animals have been limited by the inability of Vitaxin® to bind to αᵥβ₃ on rat and mouse cells. Provided below are analyses demonstrating Vitaxin® binding to common laboratory species including hamster rabbit, guinea pig and monkey.

### Results

Staining of Placental Trophoblasts

| Species | LM609 | Humanized anti-αᵥβ₃ | Vitaxin ® |
|---|---|---|---|
| Human | ND | 1-2+ | 3-1+ |
| Cynomolgus monkey | ND | 2-3+ | 3-1+ |
| Guinea pig | ND | Negative | 1-2+ |
| Hamster | ND | Negative | 1-2+ |
| Mouse | ND | Negative | Negative |
| Rabbit | 3+ | Negative | 2-3+ |
| Rat | ND | Negative | Negative |

TABLE 3
Immumohistochemical staining of placental trophoblasts.

| | | | Fold Increase Over Control | | | |
|---|---|---|---|---|---|---|
| | | | | Humanized | | |
| Species | Cell Line | Cell Type | LM609 | anti-αᵥβ₃ | Vitaxin ® | F11 |
| Human | M21 | Melanoma | 65 | 120 | 73 | 0.99 |
| | HMVEC | Endothelial | 4.3 | 6.5 | 8.9 | ND |
| Rat | RG2 | Glioma | 2.3 | 0.84 | 0.95 | 11 |
| Rabbit | VX7 | Carcinoma | 3.0 | 1.2 | 17 | 1.0 |
| Hamster | CCL-49 | Melanoma | 7.2 | 1.1 | 12 | ND |

Placental tissue, a rich source of αᵥβ₃, was collected either immediately after parturition or from freshly-sacrificed, late-term pregnant animals. Tissue fragments, approximately 1 cm³, were frozen in OCT, and thin sections were stained with monoclonal antibodies (10 μg/ml) at Sierra Biomedical (Sparks, NV). Vitaxin ® recognized αᵥβ₃ expressed on human, monkey, guinea pig, hamster and rabbit placenta, while only reacted with human and monkey αᵥ/β₃. LM609 was able to bind to rabbit αᵥβ₃, an attribute that was lost upon humanization to humanized anti-human Integrinαᵥβ₃. ND, not done.

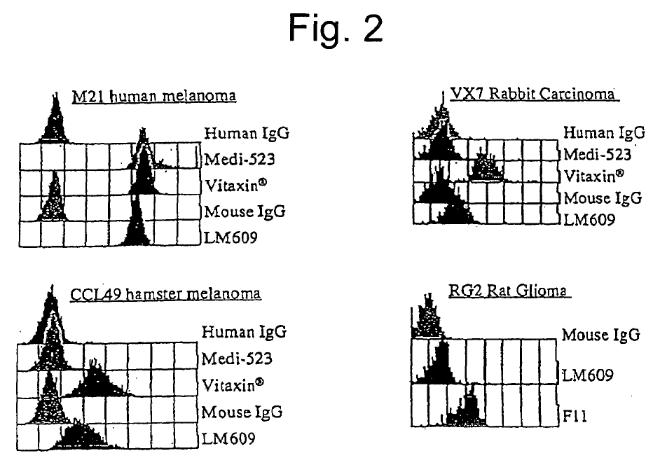

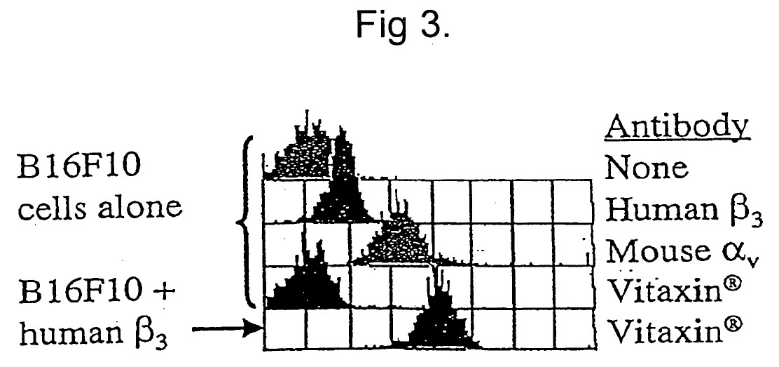

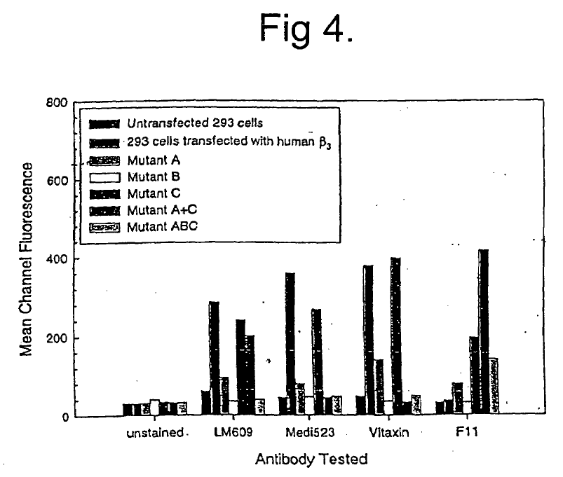

Cells (5×10⁵) were incubated with 0.5 μg of antibody and bound antibody was detected with a phycoerythrin-labeled secondary antibody. Mean channel fluorescence results are expressed as fold increase over an isotype matched control antibody. The mouse anti-rat β₃ antibody F11 was included for as a positive control for the rat cell line RG2. HMVEC, human microvascular endothelial cells; ND, not done. See FIGS. 2-4.

I¹²⁵-labeled Vitaxin® was used to determine binding affinities and number of binding sites for human, rabbit and hamster cell lines. The affinity of Vitaxin® for hamster αᵥβ₃ was approximately 2-3 fold lower than human αᵥβ₃ while its affinity for rabbit αᵥβ₃ was about 4-10 fold lower than human αᵥβ₃—See FIGS. 2-4.

Sequence comparison of residues 164 to 202 of β₃ integrins from different species and design of mutations. To further characterize the binding epitope of Vitaxin®, the gene encoding β₃ was cloned and sequenced from hamster cDNA. The amino acids in the region previously proposed to contain the LM609 binding epitope are shown above (dashed lines depict conserved residues). To determine which residues in the human sequence were essential for antibody binding, the amino acids shown in color were mutated to the corresponding rat residues. The resulting genes were transfected to HEK293 cells which express endogenous human αᵥ. Resulting cell lines were analyzed by flow cytometry for antibody binding.

TABLE 4

| Species | Cell Line | Cell Type | Kp(nM) | Binding Sites |
|---|---|---|---|---|
| Human | M21 | Melanoma | 2.2 | 1.3 × 10⁵ |
| | HUVEC | Primary endothelium | 3.3 | 8.1 − 10⁴ |
| Rabbit | RK1 | Kidney epithelium | 23 | 4.2 × 10⁴ |
| | VX7 | Carcinoma | 8.9 | 1.6 × 10⁵ |
| Hamster | CCL-49 | Melanoma | 6.9 | 2.3 × 10¹ |
| | 164 | 177 184 | | 202 |
| | : | | : : | : |
| Human | YMYISPPEALENP | CYDMKTTC | LPMFGYKHVLTLTDQVTR | (SEQ ID NO:11) |
| Hamster | | --F------K-- | --S---S- | ------------------- |
| | | | | (SEQ ID NO:12) |
| Rabbit | | ----------R-- | --------- | ------------------- |
| | | | | (SEQ ID NO:13) |
| Chicken | | --------IK-- | --EIGEK- | ---------------E-M- |
| | | | | (SEQ ID NO:14) |
| Rat | | --F-----Q-IK-- | --T--S-- | -------------------- |
| | | | | (SEQ ID NO:15) |
| Mouse | | ------Q-IK-- | --N--NA- | --------------- |
| | | | | (SEQ ID NO:16) |

| | |
|---|---|
| Mutant A | E171Q |
| Mutant B | L173I E174K |
| Mutant C | D179T T182S |
| Mutant A + C | E171Q D179T T182S |
| Mutant ABC | E171Q L1731 E174K D179T T182S |

Vitaxin® and LM609, but not another Humanized anti-α_{V}β₃, recognized hamster and rabbit αᵥβ₃ in addition to human αᵥβ₃ thereby allowing modeling of tumor angiogenesis with these antibodies in these animal species. Vitaxin®'s affinity for hamster αᵥβ₃ is approximately 2-3 fold less than for human αᵥβ₃, while its affinity for rabbit αᵥβ₃ was 4-10 fold less than for human αᵥβ₃-Vitaxin® bound to human β₃ complexed to mouse αᵥ.

Further to the above studies, Integrin αᵥβ₃ subunits were substituted as follows and analyzed by FACS analysis of the binding of Vitaxin® and/or LM609 to Integrin αᵥβ₃

Protocol for FACS analysis:
FACS analysis of HEK-293 transfectants

### Purpose:

This protocol is used to assess the surface expression of integrins in HEK-293 cells transfected with various Integrin subunits. Primary antibodies bound to the integrins are detected by fluorochrome-conjugated secondary antibodies and analyzed by flow cytometry.

### Reagents:

1. FACS buffer: PBS/2% heat-inactivated FCS/0.2% NaN₃
2. Vitaxin®, humanized anti-human_v_{—}3 Integrin mAb
3. F11, Mouse anti-rat b₃ mAb
4. P3G8, mouse anti-human_α_{V} mAb, (Chemicon, #MAB1953)
7 7, mouse anti-human_α_{V} mAb (Santa Cruz, #sc-9969)
5. P2W7, mouse anti-human β₃ mAb (Chemicon, #MAB1381)
6. 23/C6, mouse anti-human_α_{V}β₃ integrin mAb (Santa Cruz, #sc-7312)
7. LM609, mouse anti-human_α_{V}β₃ Integrin mAb
6. Goat anti-human IgG, (Fab=)₂ fragment/Alex488 conjugate (Molecular probes 4A-11013)
7. Goat anti-mouse IgG, (Fab=)₂ fragment/Alexa488 conjugate (Molecular Probes #A-11017)

### 8. Human IgG (Jackson, #009-000-003).

### 9. Mouse IgG (Jackson, #015-000-003)

### Procedures:

1. Wash adherent cells twice with 5 ml of PBS.
2. Add 1.5 ml of trypsin solution or cell dissociation solution, incubate at 37_C for 2 min.
3. Add 3 ml of culture medium or FACs buffer to the plate and loosen cells from the plate by pipetting. Count cells. Resuspend cells in FACs buffer at 5×10⁶ per ml.
4. Transfer an aliquot of 5×10⁵ cells (100 μl) into microfuge tubes containing 10 μg mouse IgG or human IgG. Incubate cells for 20 min at RT to block IgG receptor sites. Note: use mouse IgG for blocking when using human primary mAb; use human IgG for blocking when using mouse primary mAb.
5. Add 0.5 mg of Vitaxin®, or other primary mAb, and incubate for 20 min at RT.
6. Wash twice with 500 μl of FACS buffer, spinning cells at 1500 rpm for 5 minutes.
7. Add 0.5 mg of goat anti-human IgG Alexa 488, or other secondary antibodies, and incubate for 20 min at RT.
8. Wash twice with 500 μl FACS buffer, spinning cells at 1500 rpm for 5 minutes.
9. Resuspend in 500 μl buffer for FACS analysis.
10. Use unstained cells for background. For negative control, use secondary antibody alone.

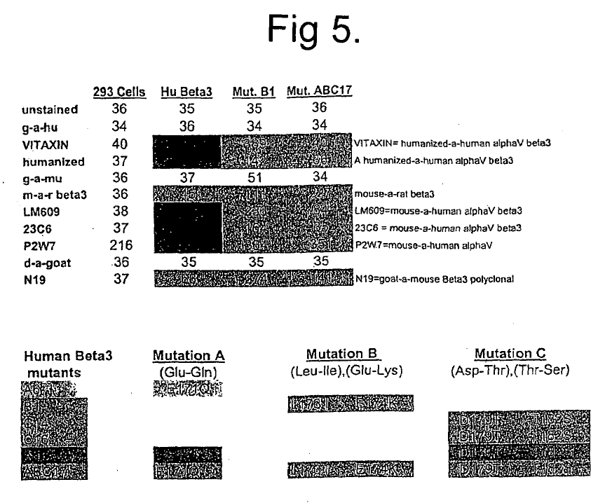

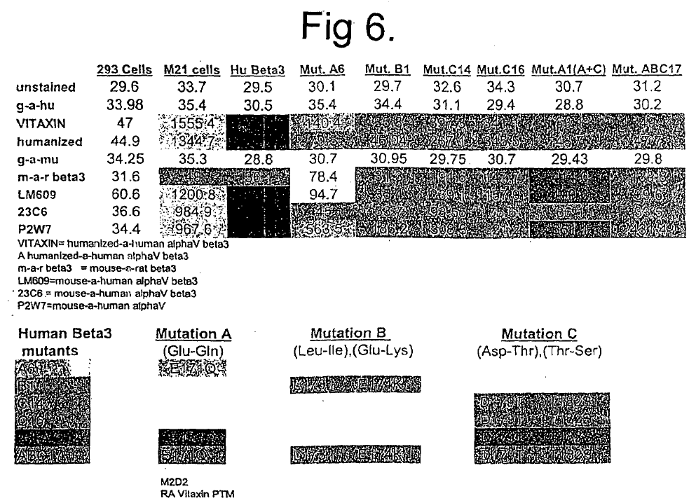

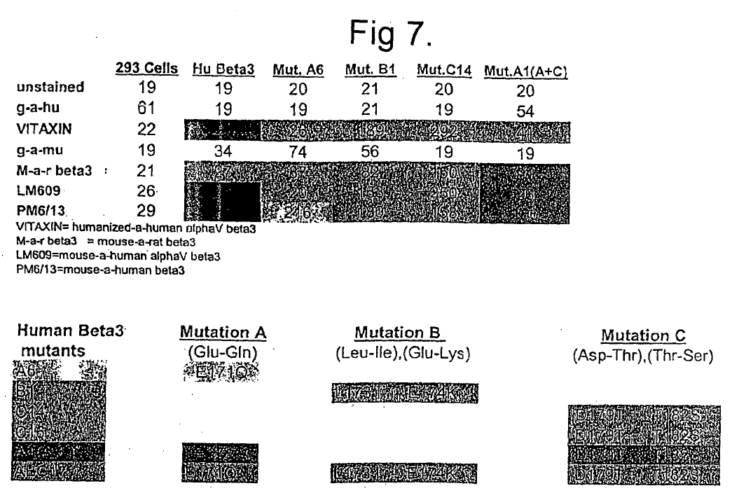

The specific substituted Integrin αᵥβ₃ mutants made are summarized in Table 1 and FIGS. 5 and 7. The binding of affinity of humanized anti-Integrin α_{V}β₃ antibodies such as Vitaxin® as well as LM609 antibodies and anti-mouse antibodies to various substituted Integrin αᵥβ₃ mutants was analyzed by FACS and the results can be summarized as seen in FIGS. 5, 6, and 7.

### EXAMPLE 3

### Immunohistochemical Procedures for Staining of the Integrin αVβ₃ in Paraffin-Embedded Tissue Sections

Immunohistochemical detection of Integrin α_{V}β₃ is effected using LM609 antibody. A number of parameters were tested for an optimal method allowing visualization of Integrin αᵥβ₃ using immunohistochemical staining of Integrin α_{V}β₃ in paraffin embedded tissue and are described as follows.

### Procedures

Fixatives and tissue processing reagents used were as follows: 10% neutral buffered formalin, OminiFix 2000, STF, Paraformaldehyde, 37% (used at 4%), Paraffin, Propar, Ethanol, 200 proof (for fixation), and Ethanol, histology grade (for processing).

Tissue processing procedures (steps following initial incubation in different fixatives) were as follows:

| Reagent | Time and Temperature |
|---|---|
| H₂O | 1 hour, room temperature |
| 70% EtOH | 30 minutes, room temperature |
| 95% EtOH | 30 minutes, room temperature |
| 95% EtOH | 30 minutes, room temperature |
| 100% EtOH | 30 minutes, room temperature |
| 100% EtOH | 30 minutes, room temperature |
| 100% EtOH | 30 minutes, room temperature |
| Propar | 60 minutes, room temperature |
| Propar | 60 minutes, room temperature |
| Propar | 60 minutes, room temperature |
| Paraffin | 10 minutes, 58 [symbol] C. |
| Paraffin | 10 minutes, 58 [symbol] C. |
| Paraffin | 10 minutes, 58 [symbol] C. |
| Paraffin | 10 minutes, 58 [symbol] C. |
| Paraffin | 10 minutes, 58 [symbol] C. |

Tissue staining reagents were as follows:

| | |
|---|---|
| Primary antibody | LM609 (Chemical) Mouse anti-human αᵥβ₃ |
| Control antibody | EBM11 (Dako) Mouse anti-human CD68 |
| Secondary antibody/ | Alkaline phosphatase anti-mouse conjugate |
| detection | with new fuschin (Signal Pathology Systems) kit |
| Blocking | Super block (Pierce) |

### Results:

Selection of fixation method: Staining of neonatal human foreskin tissue (obtained from the Cooperative Human Tissue Network) with LM609 (40 μg/ml for 2 hours at room temperature).

| Fixation Method | Results |
|---|---|
| Formalin for 24 hours | No specific vasular staining observed. +/− to |
| | 1+ squamous layer of the skin in test and in |
| | negative control. |
| Formalin for 24 hours, | +/− to 1+ staining of endotheliu observed in |
| 8 minutes microwave | some areas of both test and negative control. |
| antigen retrieval in | |
| pH 6.0 citrate | |
| | +/− to 1+ staining of squamous layer of the |
| | skin in test and in negative control. |
| Formalin for 24 hours, | +/− to 1+ staining of endotheliu observed in |
| 8 minutes microwave | some areas of both test and negative control. |
| antiben retrieval in | |
| pH 6.0 citrate | |
| | +/− to 1+ staining of squamous layer of the |
| | skin in test and in negative control. |
| OmniFix 2000 | No staining of any structures observed in |
| | either test or control. |
| STF | No specific vascular staining observed. 4+ |
| | staining of plasma cells and +/− to 2+ staining |
| | of epidermis observed in control section. |
| | Test section has +/− to 2+ staining of |
| | epidermis. |
| Paraformaldehyde | No specific vascular staining observed. |
| | Control section shows +/− to 1+ staining of |
| | the epidermis in test section. |
| 70% EtOH | Positive 2+ staining of vascular endothelium |
| | observed in test section, no vascular staining |
| | in negative control sections. −? To +/− |
| | staining of most epidermis in test and in |
| | control sections. |

The 70% EtOH fixation method was further optimized as follows. Samples of neonatal human foreskin and M21 tumor xenografts grown intradermally in human foreskin grafts on SCID mice were stained with LM609 at 10 μg/ml for 1 hour at room temperature. The sample fixed in 70% EtOH for 24 hours, 72 hours, and one week. The best staining was seen after 24 hours of fixation. Staining began to decrease when the tissue was fixed for 72 hours, and was also weaker when fixed for one week.

### EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

We claim:

1.-43. (canceled)

44. A method of preventing, treating, ameliorating, or managing renal cancer in a patient in need thereof, said method comprising administering to said patient a dose of an effective amount of an antibody or fragment thereof that immunospecifically binds Integrin αvβ3 and wherein said antibody or antibody fragment comprises a VH CDR1 (SEQ ID NO:17), VH CDR2 (SEQ ID NO:18), VH CDR3 (SEQ ID NO:19), VL CDR1 (SEQ ID NO:20), VL CDR2 (SEQ ID NO:21), and VL CDR3 (SEQ ID NO:22).

45. The method of claim 44, wherein said antibody or fragment thereof is administered to said patient concurrently with the administration of one or more other cancer therapies.

46. The method of claim 45, wherein said other cancer therapies do not include an Integrin αvβ3 antagonist.

47. The method of claim 45, wherein said other cancer therapies are chemotherapies.

48. The method of claim 47, wherein said chemotherapy comprises SU 11248.

49. The method of claim 45, wherein said other cancer therapies are biological/immunotherapies.

50. The method of claim 49, wherein said biological therapies/immunotherapies comprise bevacizumab.

51. The method of claim 44, wherein said patient has previously been treated by administration of one or more cancer therapies.

52. The method of claim 51, wherein said patient has previously been treated with chemotherapy alone or in combination with one or more radiation therapies, biological therapies/immunotherapies, hormonal therapies or surgery.

53. The method of claim 52 wherein said chemotherapy comprises SU 11248.

54. The method of claim 44, wherein said antibody or fragment thereof is administered to said subject parenterally, orally, or intratumorally.

55. The method of claim 44, wherein said antibody or fragment thereof is administered intravenously in a dose of from about 0.1 mg/kg to 10 mg/kg every week.
